(19) United States
(12) Patent Application Publication
(10) Pub. No.: US 2012/0157434 A1
(43) Pub. Date: Jun. 21, 2012

(54) Antimicrobial heterocyclic compounds for treatment of bacterial infections

(75) Inventors: Mikhail Fedorovich Gordeev, Castro Valley, CA (US); Zhengyu Yuan, Palo Alto, CA (US); Jinqian Liu, Palo Alto, CA (US)

(73) Assignee: CSO of MicuRx Pharmaceuticals, Inc.

(21) Appl. No.: 12/072,978

(22) Filed: Feb. 29, 2008

Related U.S. Application Data
(60) Provisional application No. 60/904,686, filed on Mar. 2, 2007.

(51) Int. Cl. A61K 31/4439; A61K 31/424; A61P 31/00; A61K 31/5377; A61K 31/438; C07D 498/14; C07D 498/04; A61K 31/437

(52) U.S. Cl. 514/210.21; 546/271.7; 514/338; 548/221; 514/375; 546/113; 514/300; 544/131; 514/233.2; 546/19; 514/278; 548/218

(57) ABSTRACT

The present invention provides heterocyclic compounds of the following formula I:

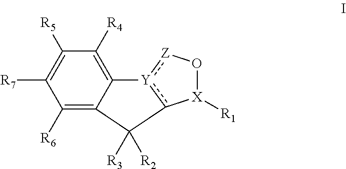

or pharmaceutically acceptable salts, prodrugs, solvates, or hydrates thereof useful as antibacterial agents, pharmaceutical compositions containing them, methods for their use, and methods for preparing these compounds.

### CROSS REFERENCE TO RELATED APPLICATIONS

The present application claims the benefit of priority of U.S. Provisional Application No. 60/904,686, filed Mar. 2, 2007, the content of which is hereby incorporated by reference in its entirety.

### FIELD OF THE INVENTION

The present invention provides novel heterocyclic derivatives of oxazolidinones, pharmaceutical compositions thereof, methods for their use, and methods for preparing of the same. These compounds have potent activities against pathogenic bacterial species.

### BACKGROUND OF THE INVENTION

Due to an increasing antibiotic resistance, novel classes of antibacterial compounds with a new mode of action are acutely needed for the treatment of bacterial infections. The antibacterials should possess useful levels of activity against certain human and veterinary pathogens, including gram-positive aerobic bacteria such as multiply-resistant staphylococci and streptococci, select anaerobes such as bacteroides and clostridia species, and acid-fast microorganisms such as Mycobacterium tuberculosis and Mycobacterium avium.

Among newer antibacterial agents, oxazolidinone compounds are the most recent synthetic class of antimicrobials active against a number of pathogenic microorganisms.

### SUMMARY OF THE INVENTION

The present invention provides compounds with useful antibacterial activity, including activity against gram-positive microorganisms.

In one aspect, the present invention provides a compound of the following formula I:

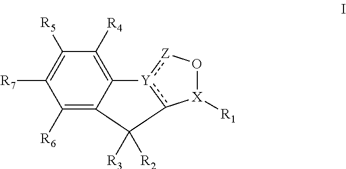

or a pharmaceutically acceptable salt, prodrug, solvate or hydrate thereof wherein:
R¹ is CH₂NHC(═O)R⁸, CONHR⁸, CHR⁸OH, CH₂NHC(═S)R⁸, CH₂NHC(═NCN)R⁸, CH₂NH-Het¹, CH₂O-Het¹, CH₂S-Het¹, CH₂Het¹, CH₂Het², and wherein R⁸ is H, NH₂ (excluding embodiments for R¹═CHR⁸OH), NHC₁₋₄alkyl (excluding embodiments for R¹═CHR⁸OH), C₃₋₆cycloalkyl, C₂₋₄alkenyl, C₂₋₄alkynyl, C₁₋₄heteroalkyl, Het¹, Het², (CH₂)ₘC(═O)C₁₋₄alkyl, OC₁₋₄alkyl (excluding embodiments for R₁═CHR⁸OH), SC₁₋₄alkyl (excluding embodiments for R₁═CHR⁸OH), (CH₂)ₚC₃₋₆cycloalkyl, (CH₂)ᵣC(═O)-aryl, or (CH₂)ₛC(═O)—Het¹; and
X is N or CH; and
Y is C, CH, or N; and
Z is C═O or N; and
R² and R³ are independently H or F; and
R⁴, R⁵, and R⁶ are independently H, F, Cl, CN, CH₃, or OH; and
R⁷ is aryl, biaryl, Het¹, Het², 4 to 7 membered heterocyclic group, such as selected from (un)substituted pyrrole, pyrroline, pyrazole, 1,2,3-triazole, 1,2,4-triazole, tetrazole, pyridine, piperidine, pyrimidine, pyridazine, pyrazine, morpholine, thiomorpholine, tetrahydrothiopyran-4-yl, piperazine, 1,4-dihydropyridone, 1,4-dihydrothiazine, azabicyclo[3.1.0]hexane, azepine, dihydroazepine, perhydroazepine, 1,4-oxazepine, 1,4-oxazepine-2-one, 1,3-oxazepine-2-one, CONHR⁸, N(C₁₋₅alkyl)CHO, N(C₁₋₅alkyl)CHet¹O, or R⁶ and R⁷ taken together are a 5 to 7-membered heterocycle;
or R⁵ and R⁷ taken together are a 5 to 7-membered heterocycle, such as selected from (un)substituted 1,3-benzoxazine, 1,4-oxazine-3-one, pyrrolidine, pyrrolidine-2-one, oxazolidine-2-one, azepine, perhydroazepine, perhydroazepine-2-one, perhydro-1,4-oxazepine, perhydro-1,4-oxazepine-2-one, perhydro-1,4-oxazepine-3-one, perhydro-1,3-oxazepine-2-one; and wherein
m, n, p, q, r and s are independently 0, 1, or 2; and wherein
dotted lines within the structure I indicate an optional double bond such that only a single double bond permitted at the group Y (i.e., the second dotted line is always omitted once the first double bond position is defined).

In another aspect, the present invention provides a compound of the following formula I:

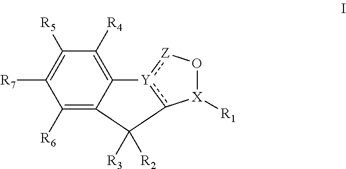

or a pharmaceutically acceptable salt, prodrug, solvate or hydrate thereof wherein:
R¹ is CH₂NHC(═O)R⁸, CONHR⁸, CH₂OH, CH₂NHC(═S)R⁸, CH₂NHC(═NCN)R⁸, CH₂NH-Het¹, CH₂O-Het¹, CH₂S-Het¹, CH₂Het¹, CH₂Het², and wherein R⁸ is H, NH₂, NHC₁₋₄alkyl, C₁₋₄alkyl, C₃₋₆cycloalkyl, C₂₋₄alkenyl, C₂₋₄alkynyl, C₁₋₄heteroalkyl, Het¹, Het², (CH₂)ₘC(═O)C₁₋₄alkyl, OC₁₋₄alkyl, SC₁₋₄alkyl, (CH₂)ₚC₃₋₆cycloalkyl, (CH₂)ᵣC(═O)-aryl, or (CH₂)ₛC(═O)—Het¹; and
X is N or CH; and
Y is C, CH, or N; and
Z is C═O or N; and
R² and R³ are independently H or F; and
R⁴, R⁵, and R⁶ are independently H, F, Cl, CN, CH₃, or OH; and
R⁷ is aryl, biaryl, Het¹, Het², or a 4 to 7 membered heterocyclic group, such as selected from (un)substituted pyrrole, pyrroline, pyrazole, 1,2,3-triazole, 1,2,4-triazole, tetrazole, pyridine, piperidine, pyrimidine, pyridazine, pyrazine, morpholine, thiomorpholine, tetrahydrothiopyran-4-yl, piperazine, 1,4-dihydropyridone, 1,4-dihydrothiazine, azabicyclo[3.1.0]hexane, azepine, dihydroazepine, perhydroazepine, 1,4-oxazepine, 1,4-oxazepine-2-one, 1,3-oxazepine-2-one, CONHR⁸, N(C₁₋₅alkyl)CHO, N(C₁₋₅alkyl)CHet¹O, or R⁶ and R⁷ taken together are a 5 to 7-membered heterocycle;
or R⁵ and R⁷ taken together are a 5 to 7-membered heterocycle, such as selected from (un)substituted 1,3-benzoxazine, 1,4-oxazine-3-one, pyrrolidine, pyrrolidine-2-one, oxazolidine-2-one, azepine, perhydroazepine, perhydroazepine-2-one, perhydro-1,4-oxazepine, perhydro-1,4-oxazepine-2-one, perhydro-1,4-oxazepine-3-one, perhydro-1,3-oxazepine-2-one; and wherein
m, n, p, q, r and s are independently 0, 1, or 2; and wherein
dotted lines within the structure I indicate an optional double bond such that only a single double bond permitted at the group Y (i.e., the second dotted line is always omitted once the first double bond position is defined).

In certain embodiments, when X is C, Z is CO, Y is N, R² is H, R³ is H, and R⁴ is H, then R⁷ is other than phenyl or substituted phenyl. In certain embodiments according to this paragraph, said substitution is any substitution apparent to those of skill in the art, for instance any radical other than hydrogen. In certain embodiments according to this paragraph, said substitution is any phenyl substitution described in U.S. Pat. No. 5,231,188, the content of which is hereby incorporated by reference in its entirety.

In certain embodiments, when X is C, Z is CO, Y is N, R¹ is CH₂NHCOR′, wherein R′ is selected from H, C₁₋₁₂alkyl (optionally substituted with 1-3 Cl), CH₂OH, CH₂OC₁₋₁₂alkyl, C₃₋₁₂cycloalkyl, phenyl (optionally substituted with 1-3 of groups OH, OMe, OEt, NO₂, halo, COOH, SO₃H, or NR″R′″ (wherein R″ and R′″ are selected from H or C₁₋₁₂alkyl)), furanyl, tetrahydrofuranyl, 2-thiophene, pyrrolidinyl, pyridinyl; OC₁₋₁₂alkyl, NH₂, NHC₁₋₁₂alkyl, NHPh, COPh; and R² is H, R³ is H, and R⁴ is H, then R⁷ is other than phenyl or phenyl substituted with CN, —C≡CH, —C≡CCMe, —C≡CCCH₂OH, N₃, NO₂, OC₁₋₄alkyl, COOH, SO₃H, halo, NH₂, NR″R′″, NR″Ph, and R⁷ is other than 1-pyrrolidyl, or R⁷ is other than COC₁₋₄alkyl. In certain embodiments according to this paragraph, said substitution is any phenyl substitution described in U.S. Pat. No. 5,231,188, the content of which is hereby incorporated by reference in its entirety.

In certain embodiments, when X is C, Z is CO, Y is N, R² is H, R³ is H, and R⁴ is H, then R⁷ is selected from biaryl, Het¹-heteroaryl, Het²-heteroaryl, Het¹, Het², a 4 to 7 membered heterocyclic group, and phenyl substituted with CH₂NHR⁷, or R⁶ and R⁷ taken together are a 5 to 7-membered heterocycle.

In certain embodiments, when X is C, Z is CO, Y is N, R¹ is CH₂COR′, wherein R′ is selected from H, C₁₋₁₂alkyl (optionally substituted with 1-3 Cl), CH₂OH, CH₂OC₁₋₁₂-alkyl, C₃₋₁₂cycloalkyl, phenyl (optionally substituted with 1-3 of groups OH, OMe, OEt, NO₂, halo, COOH, SO₃H, or NR″R′″ (wherein R″ and R′″ are selected from H or C₁₋₁₂alkyl)), furanyl, tetrahydrofuranyl, 2-thiophene, pyrrolidinyl, pyridinyl; OC₁₋₁₂alkyl, NH₂, NHC₁₋₁₂alkyl, NHPh, COPh; and R² is H, R³ is H, and R⁴ is H, then R⁷ is other than 3-pyridyl or 3-pyridyl substituted with H, C₁₋₄alkyl, NO₂, NH₂, NHC(═O)C₁₋₄alkyl, CN, COOH, OC₁₋₄alkyl, halo, or N-oxides thereof.

The alkyl, alkenyl, or cycloalkyl groups at each occurrence above independently are optionally substituted with one, two, or three substituents selected from the group consisting of halo, aryl, Het¹, and Het². Het¹ at each occurrence is independently a C-linked 5 or 6 membered heterocyclic ring having 1 to 4 heteroatoms selected from the group consisting of oxygen, nitrogen, and sulfur within the ring. Het² at each occurrence is independently a N-linked 5 or 6 membered heterocyclic ring having 1 to 4 nitrogen and optionally having one oxygen or sulfur within the ring.

In one embodiment, R⁷ in formula I is selected from azetidin-1-yl, cyclobutyl, tetrahydrothiopyranyl, tetrahydrothipyranyl sulfoximine, 1,2,5-triazacycloheptyl, 1,2,5-oxadiazacycloheptyl, 4-hydroxy-(4-methoxymethyl)piperidin-1-yl, [4-(alkylamino)methyl]phenyl, [4-(heteroaryl)alkyl)amino)methyl]phenyl, [4-((alkyl)heteroaryl)alkyl)amino)methyl]phenyl, [4-(3-fluoropropylamino)methyl]phenyl, [4-(3,3,3-trifluoro-2-hydroxypropylamino)methyl]phenyl.

In one embodiment, the compounds of formula I are selected from structures of formulas II-V below.

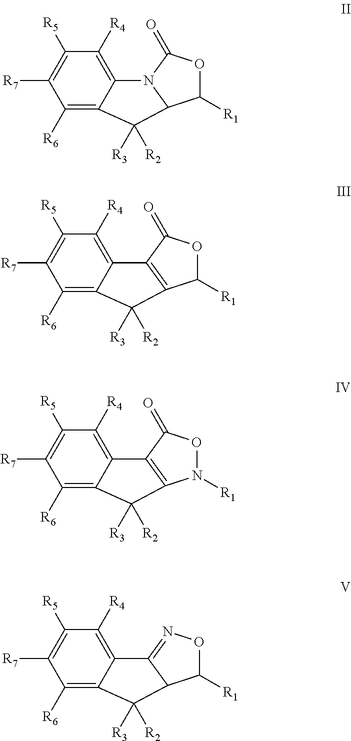

In another embodiment, the compounds of formulas II, III, and V are selected from structures of formulas VI-VIII.

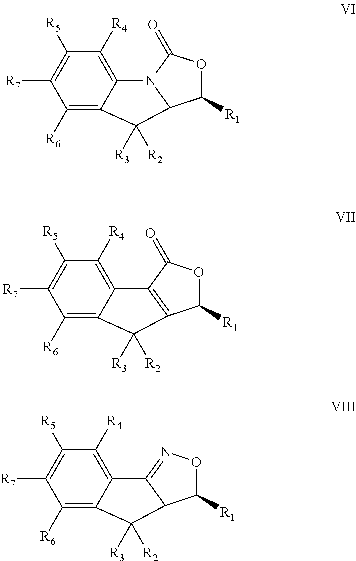

In another embodiment, the compounds of formulas VI-VIII are selected from structures of formulas IX-XI.

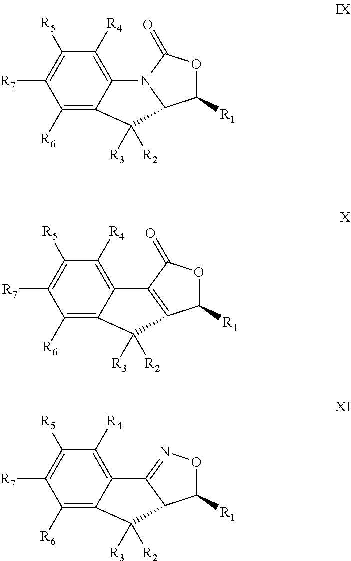

In another embodiment, provided herein are compounds according to a formula selected from the group consisting of formulas XII-XXVI.

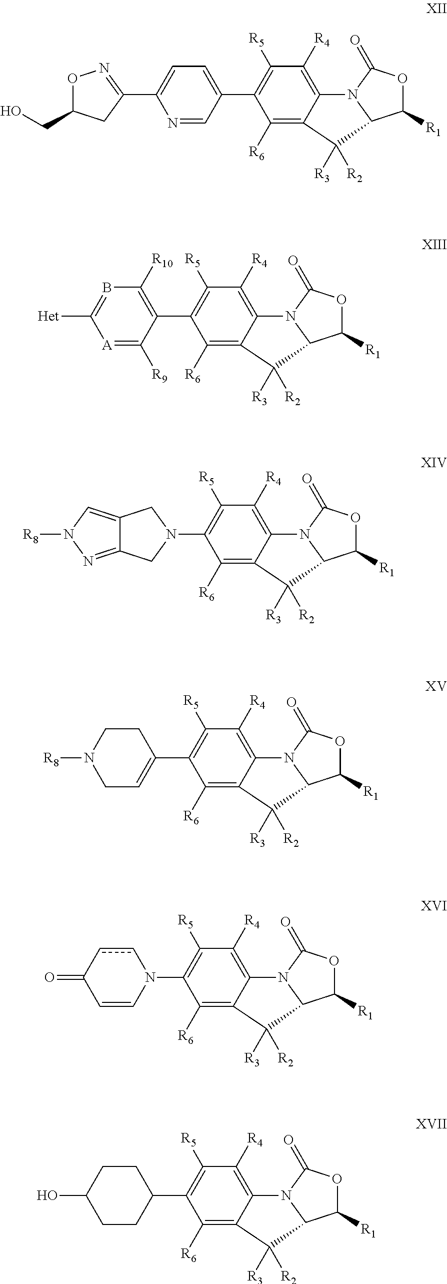

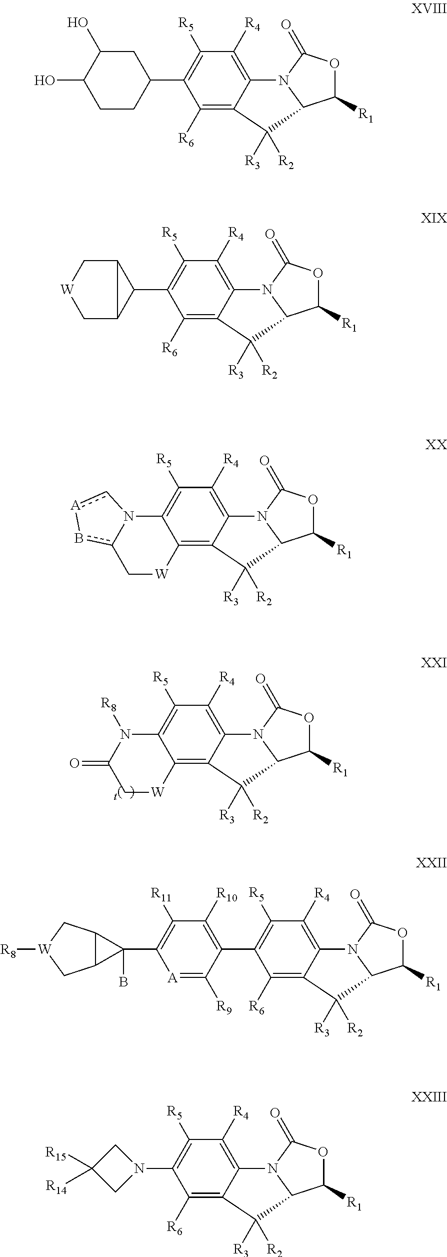

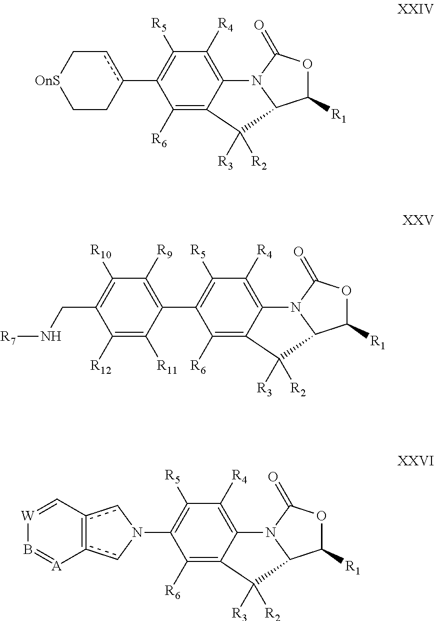

In another embodiment, the group R⁷ in compounds of formulas II-XI is selected from the following structures (wherein, straight horizontal line depicts the connection of R⁷ to an aromatic ring of the general structure I):

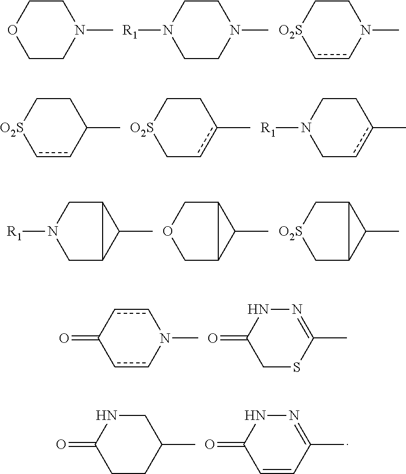

In another embodiment, the group R⁷ in compounds of formulas II-XI is selected from the following structures:

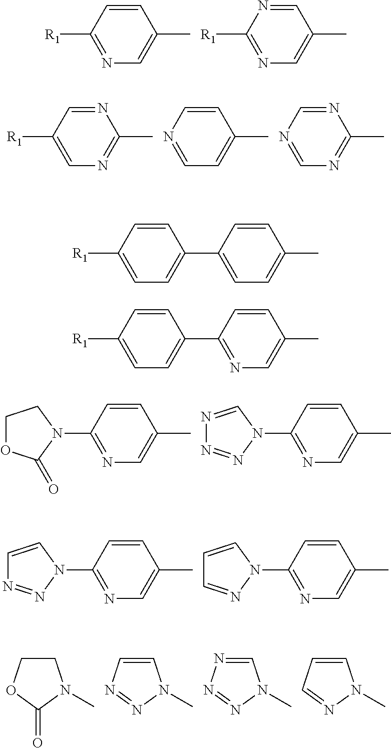

In another embodiment, compounds of formulas I, II, VI and IX are selected from the following structures:

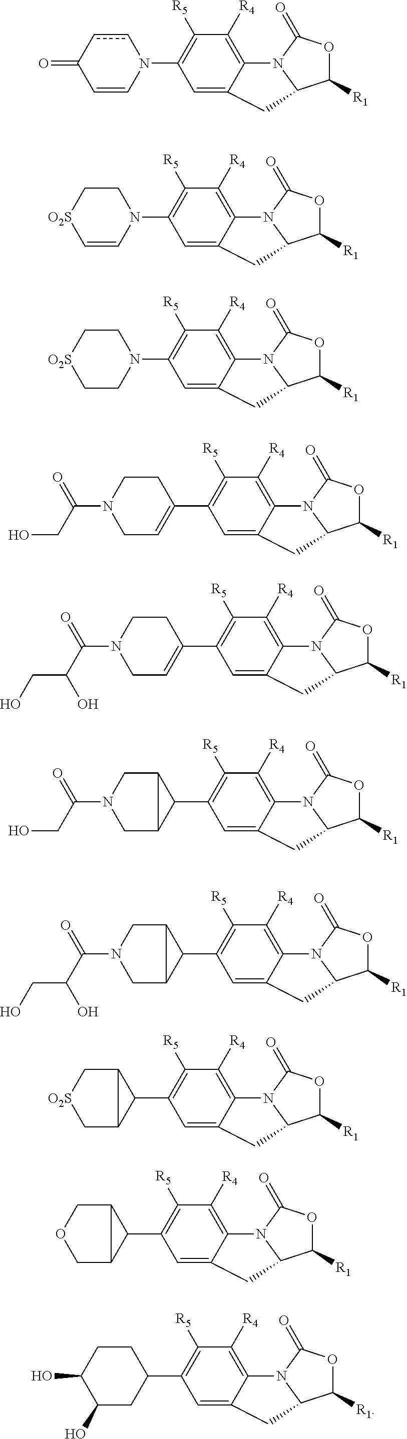

In another embodiment, compounds of formulas I, II, VI and IX are selected from the following structures:

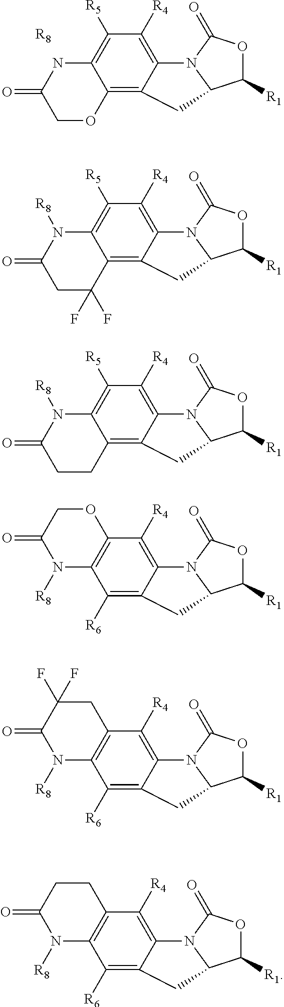

In another embodiment, compounds of formulas I, II, VI and IX are selected from the following structures:

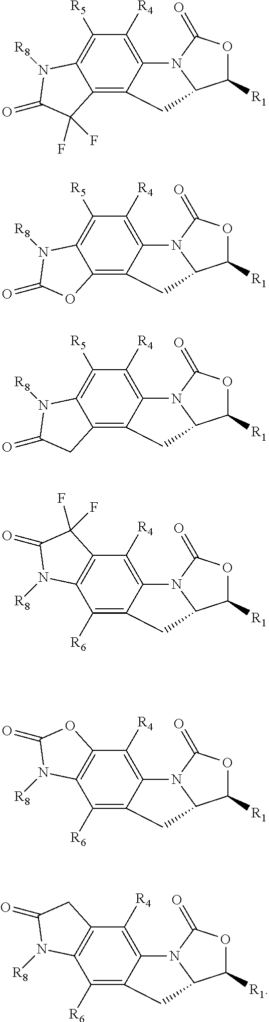

In another embodiment, compounds of formulas I, II, VI and IX are selected from the following structures:

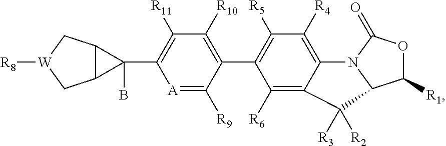

wherein A is N or C—R¹²; W is H, O, S(O)ₙ, or N; and B, R⁹, R¹⁰, R¹¹, and R¹² are independently H, halo, F, CN, CH₃, or OH.

In another embodiment, compounds of formulas I, II, VI and IX are selected from the following structures:

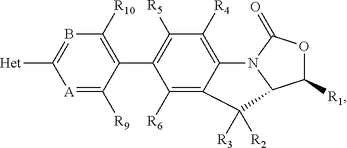

wherein A and B are independently N or C—R¹², or C—R¹³; Het is Het₁ or Het₂; and R⁹, R¹⁰, R¹¹, R¹², R¹³ are independently H, halo, F, CN, CH₃, or OH.

In another embodiment, compounds of formulas I, II, VI and IX are selected from the following structures:

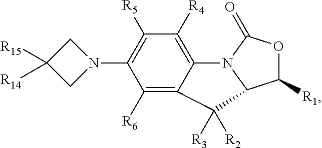

wherein R¹³ and R¹⁴ are independently H, halo, F, CN, C₁₋₄alkyl, OC₁₋₄alkyl, or OH; or wherein R¹³ and R¹⁴ taken together are ═O, ═S, ═N—OH, ═N—OC₁₋₄alkyl, or ═N—CN.

In another embodiment, compounds of formulas I, II, VI and IX are selected from the following structures:

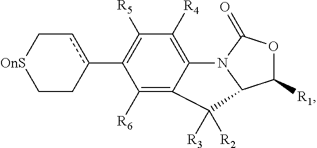

wherein a dotted line is either a single bond or a double bond.

In another embodiment, compounds of formulas I, II, VI and IX are selected from the following structures:

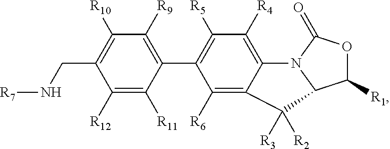

wherein and R⁹, R¹⁰, R¹¹, and R¹² are independently H, halo, F, CN, CH₃, or OH.

In another aspect, the present invention provides a pharmaceutical composition comprising a compound of formula I, or a pharmaceutically acceptable salt, prodrug, solvate, or hydrate thereof, and a pharmaceutically acceptable carrier, excipient or diluent.

In an additional aspect, the present invention provides a method for treating gram-positive microbial infections in humans or other warm-blooded animals by administering to the subject in need a therapeutically effective amount of a compound of formula I or a pharmaceutically acceptable salt, prodrug, solvate, or hydrate thereof. The compound of formula I may be administered orally, parenterally, transdermally, topically, rectally, or intranasally.

In yet another aspect, the present invention provides novel intermediates and processes for preparing compounds of formula I.

### DETAILED DESCRIPTION OF THE INVENTION

Unless otherwise stated, the following terms used in the specification and Claims have the meanings given below:

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₇ alkyl refers to alkyl of one to seven carbon atoms, inclusive.

Unless indicated otherwise, superscripts and subscripts are interchangeable. I.e., R⁸ is the same as R₈, and Het¹ is the same as Het₁, etc.

The terms “alkyl,” “alkenyl,” etc. refer to both straight and branched groups, but reference to an individual radical such as “propyl” embraces only the straight chain radical, a branched chain isomer such as “isopropyl” being specifically referred to. The alkyl, alkenyl, etc. group may be optionally substituted with one, two, or three substituents selected from the group consisting of halo, aryl, Het¹, or Het². Representative examples include, but are not limited to, difluoromethyl, 2-fluoroethyl, trifluoroethyl. —CH═CH-aryl, —CH═CH-Het¹, —CH₂-phenyl, and the like.

The term “optionally substituted” is intended to mean that a group, such as an alkyl, alkylene, alkenyl, alkenylene, alkynyl, alkynylene, cycloalkyl, cycloalkylene, aryl, arylene, heteroaryl, heterocyclyl group, alkoxy, or acyl, may be substituted with one or more substituents independently selected from, e.g., alkyl, alkylene, alkenyl, alkenylene, alkynyl, alkynylene, cycloalkyl, cycloalkylene, aryl, arylene, heteroaryl, or heterocyclyl, each optionally substituted with one or more, in one embodiment, one, two, three, or four, substituents Q; halo, cyano (—CN), nitro (—NO₂), —SR^{a}, —S(O)R^{a}, —S(O)₂R^{a}, —R^{a}, —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{b}, —NR^{a}C(O)OR^{b}, —NR^{a}C(O)NR^{b}R^{c}, —NR^{a}C(═NR^{d})NR^{b}R^{c}, —NR^{a}S(O)R^{b}, —NR^{a}S(O)₂R^{b}, —NR^{a}S(O)R^{b}R^{c}, or —NR^{a}S(O)₂R^{b}R^{c}; wherein each R^{a}, R^{b}, R^{c}, and R^{d} is independently hydrogen; C₁₋₆ alkyl, C₂₋₆ alkenyl, C₂₋₆ alkynyl, C₃₋₇ cycloalkyl, C₆₋₁₄ aryl, heteroaryl, or heterocyclyl, each optionally substituted with one or more, in one embodiment, one, two, three, or four, substituents Q; or R^{b} and R^{c} together with the N atom to which they are attached form heterocyclyl or heteroaryl, each optionally substituted with one or more, in one embodiment, one, two, three, or four, substituents Q. As used herein, all groups that can be substituted in one embodiment are “optionally substituted,” unless otherwise specified.

The term “cycloalkyl” means a cyclic saturated monovalent hydrocarbon group of three to six carbon atoms, e.g., cyclopropyl, cyclohexyl, and the like. The cycloalkyl group may be optionally substituted with one, two, or three substituents selected from the group consisting of halo, aryl, Het¹, or Het².

The term “heteroalkyl” means an alkyl or cycloalkyl group, as defined above, having a substituent containing a heteroatom selected from N, O, or S(O)ₙ, where n is an integer from 0 to 2, including, hydroxy (OH), C₁₋₄alkoxy, amino, thio (—SH), and the like. Representative substituents include —NRₐR_{b}, —ORₐ, or —S(O)ₙR_{c}, wherein Rₐ is hydrogen, C₁₋₄alkyl, C₃₋₆cycloalkyl, optionally substituted aryl, optionally substituted heterocyclic, or —COR (where R is C₁₋₄alkyl); R_{b} is hydrogen, C₁₋₄alkyl, —SO₂R (where R is C₁₋₄alkyl or C₁₋₄hydroxyalkyl), —SO₂NRR′ (where R and R′ are independently of each other hydrogen or C₁₋₄alkyl), —CONR′R″ (where R′ and R″ are independently of each other hydrogen or C₁₋₄alkyl); n is an integer from 0 to 2; and R_{c} is hydrogen, C₁₋₄alkyl, C₃₋₆cycloalkyl, optionally substituted aryl, or NRₐR_{b}, where Rₐ and R_{b} are as defined above. Representative examples include, but are not limited to 2-methoxyethyl (—CH₂CH₂OCH₃), 2-hydroxyethyl (—CH₂CH₂OH), hydroxymethyl(—CH₂OH), 2-aminoethyl (—CH₂CH₂NH₂), 2-dimethylaminoethyl (—CH₂CH₂NHCH₃), benzyloxymethyl, thiophen-2-ylthiomethyl, and the like.

The term “halo” refers to fluoro (F), chloro (Cl), bromo (Br), or iodo (I).

The term “aryl” refers to phenyl, biphenyl, or naphthyl, optionally substituted with 1 to 3 substituents independently selected from halo, —C₁₋₄alkyl, —OH, —OC₁₋₄alkyl, —S(O)ₙC₁₋₄alkyl wherein n is 0, 1, or 2, —C₁₋₄alkylNH₂, —NHC₁₋₄alkyl, —C(═O)H, or —C═N—OR_{d} wherein R_{d} is hydrogen or —C₁₋₄alkyl.

The term “heterocyclic ring” refers to an aromatic ring or a saturated or unsaturated ring that is not aromatic of 3 to 10 carbon atoms and 1 to 4 heteroatoms selected from the group consisting of oxygen, nitrogen, and S(O)ₙ within the ring, where n is defined above. The heterocyclic ring may be optionally substituted with halo, —C₁₋₄alkyl, —OH, —OC₁₋₄ alkyl, —S(O)ₙC₁₋₄alkyl wherein n is 0, 1, or 2, —C₁₋₄alkylNH₂, —NHC₁₋₄alkyl, —C(═O)H, or —C═N—OR_{d} wherein R_{d} is hydrogen or C₁₋₄alkyl.

Examples of heterocylic rings include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isoxazolinone, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiadiazole tetrazole, thiazolidine, thiophene, benzo[b]thiophene, morpholinyl, thiomorpholinyl (also referred to as thiamorpholinyl), piperidinyl, pyrrolidine, tetrahydrofuranyl, 1,3-benzoxazine, 1,4-oxazine-3-one, 1,3-benzoxazine-4-one, pyrrolidine, pyrrolidine-2-one, oxazolidine-2-one, azepine, perhydroazepine, perhydroazepine-2-one, perhydro-1,4-oxazepine, perhydro-1,4-oxazepine-2-one, perhydro-1,4-oxazepine-3-one, perhydro-1,3-oxazepine-2-one and the like. Heterocyclic rings include substituted and substituted rings.

Specifically, Het¹ (same as het¹) refers to a C-linked five- (5) or six- (6) membered heterocyclic ring. Representative examples of “Het¹” include, but are not limited to, pyridine, thiophene, furan, pyrazole, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxaz-olyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,5-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl and 5-isothiazolyl, 1,3,4,-oxadiazole, 4-oxo-2-thiazolinyl, or 5-methyl-1,3,4-thiadiazol-2-yl, thiazoledione, 1,2,3,4-thiatriazole, or 1,2,4-dithiazolone.

Het² (same as het²) refers to a N-linked five- (5) or six- (6) membered heterocyclic ring having 1 to 4 nitrogen atoms, and optionally having one oxygen or sulfur atom. Representative examples of “Het²” include, but are not limited to pyrrolyl, imidazolyl, pyrazolyl, 1,2,3-triazolyl, 1,2,4-triazolyl, 1,2,3,4-tetrazolyl, and isoxazolidinonyl group.

“Optional” or “optionally” means that the subsequently described event or circumstance may, but need not, occur, and that the description includes instances where the event or circumstance occurs and instances in which it does not. For example, “aryl group optionally mono- or di-substituted with an alkyl group” means that the alkyl may but need not be present, and the description includes situations where the aryl group is mono- or disubstituted with an alkyl group and situations where the aryl group is not substituted with the alkyl group.

Compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and Claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

A “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and Claims includes both one and more than one such carrier.

A “pharmaceutically acceptable salt” of a compound means a salt that is pharmaceutically acceptable and that possesses the desired pharmacological activity of the parent compound. Such salts include:
(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease,
(2) inhibiting the disease, i.e., arresting or reducing the development of the disease or its clinical symptoms, or
(3) relieving the disease, i.e., causing regression of the disease or its clinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Leaving group” has the meaning conventionally associated with it in synthetic organic chemistry, i.e., an atom or group capable of being displaced by a nucleophile and includes halogen, C₁₋₄alkylsulfonyloxy, ester, or amino such as chloro, bromo, iodo, mesyloxy, tosyloxy, trifluorosulfonyloxy, methoxy, N,O-dimethylhydroxyl-amino, and the like.

“Prodrug” means any compound which releases an active parent drug according to a compound of the subject invention in vivo when such prodrug is administered to a mammalian subject. Prodrugs of a compound of the subject invention are prepared by modifying functional groups present in a compound of the subject invention in such a way that the modifications may be cleaved in vivo to release the parent compound. Prodrugs include compounds of the subject invention wherein a hydroxy, sulfhydryl, amido or amino group in the compound is bonded to any group that may be cleaved in vivo to regenerate the free hydroxyl, amido, amino, or sulfhydryl group, respectively. Examples of prodrugs include, but are not limited to esters (e.g., acetate, formate, benzoate, phosphate or phosphonate derivatives), carbamates (e.g., N,N-dimethylaminocarbonyl) of hydroxy functional groups in compounds of the subject invention, and the like.

The term “mammal” refers to all mammals including humans, livestock, and companion animals.

The compounds of the present invention are generally named according to the IUPAC or CAS nomenclature system. Abbreviations which are well known to one of ordinary skill in the art may be used (e.g. “Ph” for phenyl, “Me” for methyl, “Et” for ethyl, “h” for hour or hours and “rt” for room temperature).

### ILLUSTRATIVE EMBODIMENTS

Within the broadest definition of the present invention, certain compounds of the compounds of formula I may be preferred. Specific and preferred values listed below for radicals, substituents, and ranges, are for illustration only; they do not exclude other defined values or other values within defined ranges for the radicals and substituents.

In some preferred compounds of the present invention C₁₋₄alkyl can be methyl, ethyl, propyl, isopropyl, butyl, iso-butyl, sec-butyl, and isomeric forms thereof.

In some preferred compounds of the present invention C₂₋₄alkenyl can be vinyl, propenyl, allyl, butenyl, and isomeric forms thereof (including cis and trans isomers).

In some preferred compounds of the present invention C₃₋₆cycloalkyl can be cyclopropyl, cyclobutyl, cyclopentyl, cyclohexyl, and isomeric forms thereof.

In some preferred compounds of the present invention C₁₋₄heteroalkyl can be hydroxymethyl, hydroxyethyl, and 2-methoxyethyl.

In some preferred compounds of the present invention halo can be fluoro (F) or chloro (Cl).

In some preferred compounds of the present invention R¹ can be CH₂C(═O)C₁₋₄alkyl or CH₂C(═O)OC₁₋₄alkyl.

In some preferred embodiments, group R¹ is selected from CH₂OH, CH(OH)CH═CH₂, or CH(OH)C≡CH.

In some preferred embodiments, group R¹ is selected from CONH₂ or CONHMe.

In some preferred embodiments, group R¹ is selected from CH₂NHC(═O)Me, CH₂NHC(═O)Et, or CH₂NHC(═O)OMe.

In some preferred embodiments, group R¹ is selected from CH₂(1,2,3-triazol-1-yl) or CH₂(4-methyl-1,2,3-triazol-1-yl).

In some preferred embodiments, group R¹ is selected from CH₂NH(isoxazol-3-yl), CH₂O(isoxazol-3-yl), CH₂NH(pyridin-2-yl), or CH₂O(pyridin-2-yl), CH₂NH(pyridin-3-yl), or CH₂O(pyridin-3-yl).

In some preferred embodiments, groups R⁴ and R⁶ are independently selected from H or F.

In some preferred embodiments, group R⁴ is H, and group R⁶ is F.

In some preferred embodiments, R⁴, R⁵ and R⁶ independently can be H or F.

In some preferred embodiments, one of R⁴ and R⁵ is H and the other is F.

In some preferred embodiments, X can be CH, Y can be N, and Z can be C═O.

In some preferred embodiments, R⁷ and R⁶ taken together are —NR⁸C(═O)CH₂O— (i.e., perhydro-1,4-oxazine-3-one fused to the aromatic ring).

In some preferred embodiments, R⁷ and R⁶ taken together are —NR⁸C(═O)CH₂O— (i.e., perhydro-1,4-oxazine-3-one fused to the aromatic ring).

In some preferred embodiments R⁷ and R⁶ taken together are —NR⁸C(═O)O— (i.e., oxazole-3-one fused to the aromatic ring).

In some preferred embodiments, R⁷ and R⁵ taken together are —NR⁸C(═O)O— (i.e., oxazole-3-one fused to the aromatic ring).

In some preferred embodiments, R⁷ and R⁶ taken together are —NR⁸C(═O)CF₂—.

In some preferred embodiments, R⁷ and R⁵ taken together are —NR⁸C(═O)CF₂—.

In some preferred embodiments, R⁷ and R⁶ taken together are —NR⁸C(═O)CH₂—.

In some preferred embodiments, R⁷ and R⁵ taken together are —NR⁸C(═O)CH₂—.

In some preferred embodiments, R⁸ can be —C₁₋₄alkyl, optionally substituted with one, two or three fluoro (F) or chloro (Cl).

In some preferred embodiments, R⁸ can be H, CH₃, CHF₂, CF₃, CHCl₂, CH₂CF₃, CH₂CH₃, CH₂CHF₂, CH₂CH₂F.

In some preferred embodiments, R⁸ can be CH₂OH, CH₂CH₂OH, or NH₂.

In some preferred embodiments, R⁸ can be CH═CH-aryl. Specifically, R⁸ can be CH═CH-Het¹ or CH═CH-Het².

In some preferred embodiments, Het¹ can be 2-pyridyl, 3-pyridyl, 4-pyridyl, 3-isoxazolyl, 4-isoxaz-olyl, 5-isoxaz-olyl, 1,2,3-triazol-1-yl, or 1,2,5-thiadiazol-3-yl group.

In some preferred embodiments, Het² can be pyrrolyl, imidazolyl, pyrazolyl, 1,2,3-triazolyl, 1,2,4-triazolyl, 1,2,3,4-tetrazolyl, and isoxazolidinonyl group.

It will also be appreciated by those skilled in the art that compounds of the present invention may have additional chiral centers and be isolated in optically active and racemic forms. The present invention encompasses any racemic, optically active, tautomeric, or stereoisomeric form, or mixture thereof, of a compound of the invention.

One preferred group of compounds of the present invention is illustrated below (wherein the bond with a dotted line represents either single or double bond).

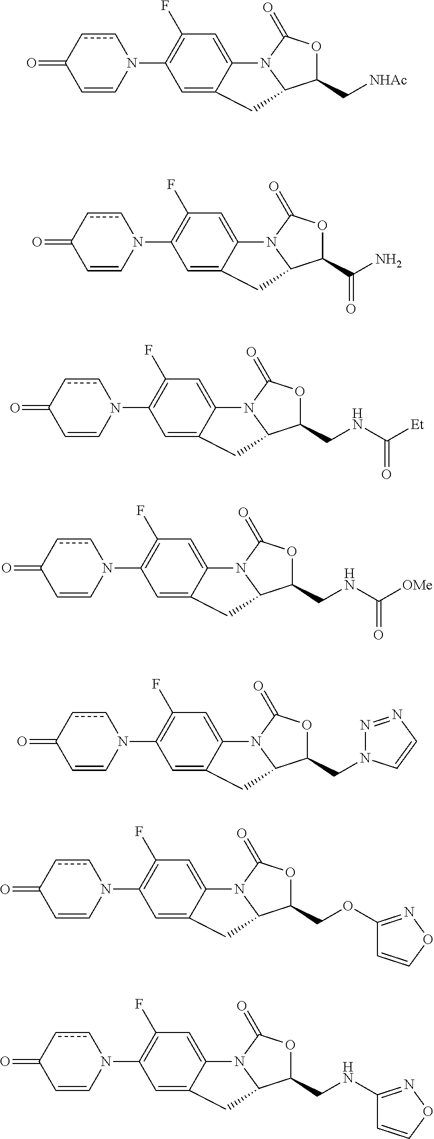

N-(((1S,9aS)-6-fluoro-3-oxo-7-(4-oxopyridin-1(4H)-yl)-1,3,9,9a-tetrahydrooxazolo[3,4-a]indol-1-yl)methyl)acetamide
N-(((1S,9aS)-6-fluoro-3-oxo-7-(4-oxo-3,4-dihydropyridin-1(2H)-yl)-1,3,9,9a-tetrahydrooxazolo[3,4-a]indol-1-yl)methyl)acetamide
(1R,9aS)-6-fluoro-3-oxo-7-(4-oxopyridin-1(4H)-yl)-1,3,9,9a-tetrahydrooxazolo[3,4-a]indole-1-carboxamide
(1R,9aS)-6-fluoro-3-oxo-7-(4-oxo-3,4-dihydropyridin-1(2H)-yl)-1,3,9,9a-tetrahydrooxazolo[3,4-a]indole-1-carboxamide
N-(((1S,9aS)-6-fluoro-3-oxo-7-(4-oxopyridin-1(4H)-yl)-1,3,9,9a-tetrahydrooxazolo[3,4-a]indol-1-yl)methyl)propionamide
N-(((1S,9aS)-6-fluoro-3-oxo-7-(4-oxo-3,4-dihydropyridin-1(2H)-yl)-1,3,9,9a-tetrahydrooxazolo[3,4-a]indol-1-yl)methyl)propionamide
methyl(((1S,9aS)-6-fluoro-3-oxo-7-(4-oxopyridin-1(4H)-yl)-1,3,9,9a-tetrahydrooxazolo[3,4-a]indol-1-yl)methyl)carbamate
methyl(((1S,9aS)-6-fluoro-3-oxo-7-(4-oxo-3,4-dihydropyridin-1(2H)-yl)-1,3,9,9a-tetrahydrooxazolo[3,4-a]indol-1-yl)methyl)carbamate
(1S,9aS)-1-((1H-1,2,3-triazol-1-yl)methyl)-6-fluoro-7-(4-oxo-3,4-dihydropyridin-1(2H)-yl)-9,9a-dihydrooxazolo[3,4-a]indol-3(1H)-one
(1S,9aS)-1-((1H-1,2,3-triazol-1-yl)methyl)-6-fluoro-7-(4-oxopyridin-1(2H)-yl)-9,9a-dihydrooxazolo[3,4-a]indol-3(1H)-one
(1R,9aS)-6-fluoro-1-((isoxazol-3-yloxy)methyl)-7-(4-oxo-3,4-dihydropyridin-1(4H)-yl)-9,9a-dihydrooxazolo[3,4-a]indol-3(1H)-one
(1R,9aS)-6-fluoro-1-((isoxazol-3-yloxy)methyl)-7-(4-oxopyridin-1(4H)-yl)-9,9a-dihydrooxazolo[3,4-a]indol-3(1H)-one
(1S,9aS)-6-fluoro-1-((isoxazol-3-ylamino)methyl)-7-(4-oxopyridin-1(4H)-yl)-9,9a-dihydrooxazolo[3,4-a]indol-3(1H)-one
(1S,9aS)-6-fluoro-1-((isoxazol-3-ylamino)methyl)-7-(4-oxo-3,4-dihydropyridin-1(2H)-yl)-9,9a-dihydrooxazolo[3,4-a]indol-3(1H)-one

Additional preferred group of compounds of the present invention is illustrated below.

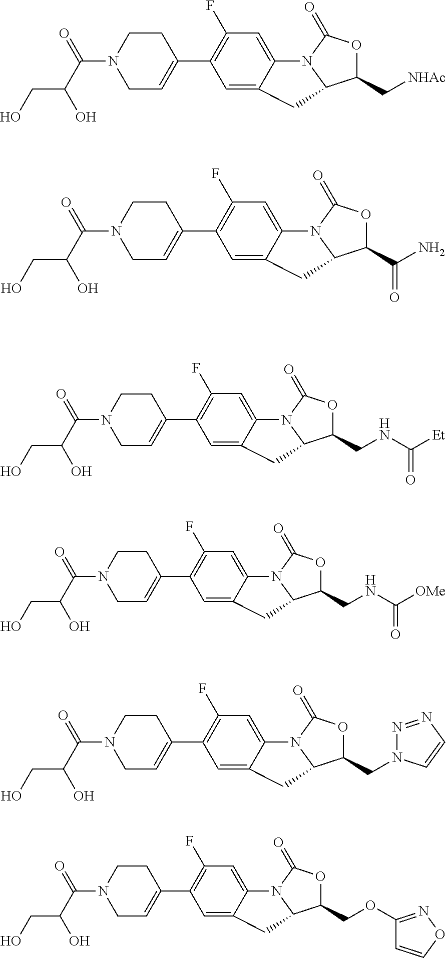

N-(((1S,9aS)-7-(1-(2,3-dihydroxypropanoyl)-1,2,3,6-tetrahydropyridin-4-yl)-6-fluoro-3-oxo-1,3,9,9a-tetrahydrooxazolo[3,4-a]indol-1-yl)methyl)acetamide
(1R,9aS)-7-(1-(2,3-dihydroxypropanoyl)-1,2,3,6-tetrahydropyridin-4-yl)-6-fluoro-3-oxo-1,3,9,9a-tetrahydrooxazolo[3,4-a]indole-1-carboxamide
N-(((1S,9aS)-7-(1-(2,3-dihydroxypropanoyl)-1,2,3,6-tetrahydropyridin-4-yl)-6-fluoro-3-oxo-1,3,9,9a-tetrahydrooxazolo[3,4-a]indol-1-yl)methyl)propionamide
methyl(((1S,9aS)-7-(1-(2,3-Dihydroxypropanoyl)-1,2,3,6-tetrahydropyridin-4-yl)-6-fluoro-3-oxo-1,3,9,9a-tetrahydrooxazolo[3,4-a]indol-1-yl)methyl)carbamate
(1S,9aS)-1-((1H-1,2,3-triazol-1-yl)methyl)-7-(1-(2,3-dihydroxypropanoyl)-1,2,3,6-tetrahydropyridin-4-yl)-6-fluoro-9,9a-dihydrooxazolo[3,4-a]indol-3(1H)-one
(1R,9aS)-7-(1-(2,3-dihydroxypropanoyl)-1,2,3,6-tetrahydropyridin-4-yl)-6-fluoro-1-((isoxazol-3-yloxy)methyl)-9,9a-dihydrooxazolo[3,4-a]indol-3 (1H)-one

Another preferred group of compounds of the present invention is illustrated below.

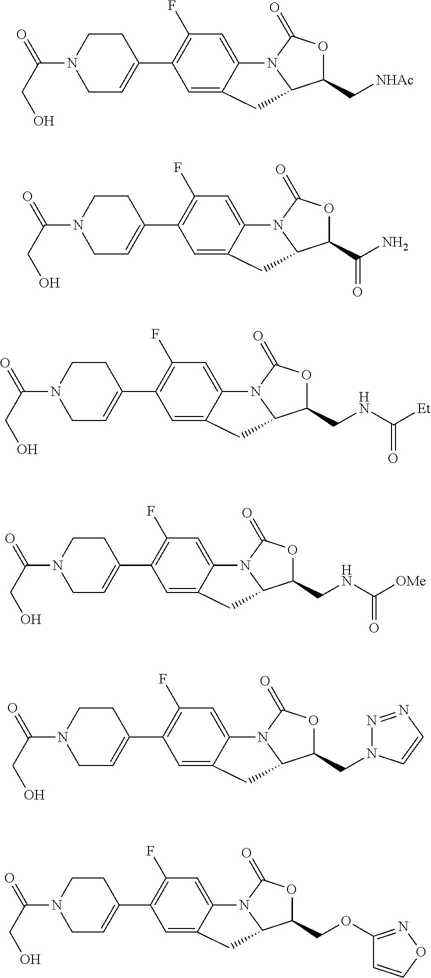

N-(((1S,9aS)-6-fluoro-7-(1-(2-hydroxyacetyl)-1,2,3,6-tetrahydropyridin-4-yl)-3-oxo-1,3,9,9a-tetrahydrooxazolo[3,4-a]indol-1-yl)methyl)acetamide
(1R,9aS)-6-fluoro-7-(1-(2-hydroxyacetyl)-1,2,3,6-tetrahydropyridin-4-yl)-3-oxo-1,3,9,9a-tetrahydrooxazolo[3,4-a]indole-1-carboxamide
N-(((1S,9aS)-6-fluoro-7-(1-(2-hydroxyacetyl)-1,2,3,6-tetrahydropyridin-4-yl)-3-oxo-1,3,9,9a-tetrahydrooxazolo[3,4-a]indol-1-yl)methyl)propionamide
methyl(((1S,9aS)-6-fluoro-7-(1-(2-hydroxyacetyl)-1,2,3,6-tetrahydropyridin-4-yl)-3-oxo-1,3,9,9a-tetrahydrooxazolo[3,4-a]indol-1-yl)methyl)carbamate
(1S,9aS)-1-((1H-1,2,3-triazol-1-yl)methyl)-6-fluoro-7-(1-(2-hydroxyacetyl)-1,2,3,6-tetrahydropyridin-4-yl)-9,9a-dihydrooxazolo[3,4-a]indol-3 (1H)-one
(1R,9aS)-6-fluoro-7-(1-(2-hydroxyacetyl)-1,2,3,6-tetrahydropyridin-4-yl)-1-((isoxazol-3-yloxy)methyl)-9,9a-dihydrooxazolo[3,4-a]indol-3 (1H)-one

Another preferred group of compounds of the present invention is illustrated below.

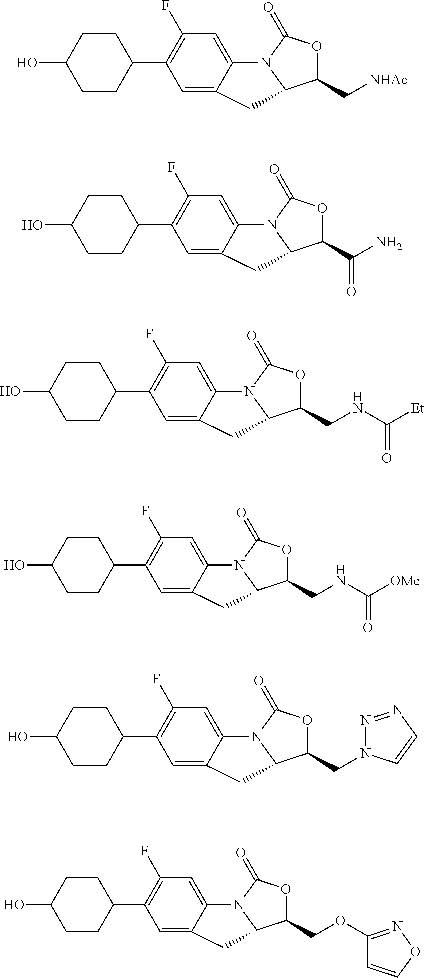

N-(((1S,9aS)-6-fluoro-7-(4-hydroxycyclohexyl)-3-oxo-1,3,9,9a-tetrahydrooxazolo[3,4-a]indol-1-yl)methyl)acetamide
(1R,9aS)-6-fluoro-7-(4-hydroxycyclohexyl)-3-oxo-1,3,9,9a-tetrahydrooxazolo[3,4-a]indole-1-carboxamide
N-(((1S,9aS)-6-fluoro-7-(4-hydroxycyclohexyl)-3-oxo-1,3,9,9a-tetrahydrooxazolo[3,4-a]indol-1-yl)methyl)propionamide
methyl(((1S,9aS)-6-fluoro-7-(4-hydroxycyclohexyl)-3-oxo-1,3,9,9a-tetrahydrooxazolo[3,4-a]indol-1-yl)methyl)carbamate
(1S,9aS)-1-((1H-1,2,3-triazol-1-yl)methyl)-6-fluoro-7-(4-hydroxycyclohexyl)-9,9a-dihydrooxazolo[3,4-a]indol-3(1H)-one
(1R,9aS)-6-fluoro-7-(4-hydroxycyclohexyl)-1-((isoxazol-3-yloxy)methyl)-9,9a-dihydrooxazolo[3,4-a]indol-3(1H)-one

Another preferred group of compounds of the present invention is illustrated below.

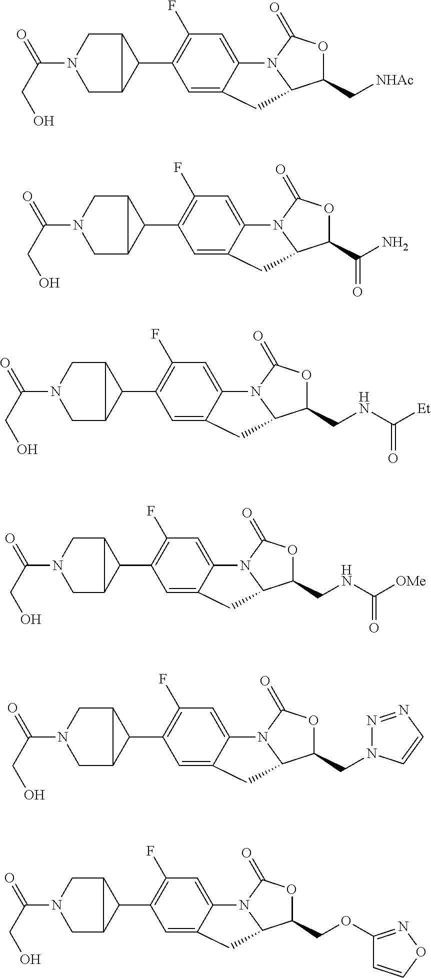

N-(((1S,9aS)-6-fluoro-7-(3-(2-hydroxyacetyl)-3-azabicyclo[3.1.0]hexan-6-yl)-3-oxo-1,3,9,9a-tetrahydrooxazolo[3,4-a]indol-1-yl)methyl)acetamide
(1R,9aS)-6-fluoro-7-(3-(2-hydroxyacetyl)-3-azabicyclo[3.1.0]hexan-6-yl)-3-oxo-1,3,9,9a-tetrahydrooxazolo[3,4-a]indole-1-carboxamide
N-(((1S,9aS)-6-fluoro-7-(3-(2-hydroxyacetyl)-3-azabicyclo[3.1.0]hexan-6-yl)-3-oxo-1,3,9,9a-tetrahydrooxazolo[3,4-a]indol-1-yl)methyl)propionamide
methyl(((1S,9aS)-6-fluoro-7-(3-(2-hydroxyacetyl)-3-azabicyclo[3.1.0]hexan-6-yl)-3-oxo-1,3,9,9a-tetrahydrooxazolo[3,4-a]indol-1-yl)methyl)carbamate
(1S,9aS)-1-((1H-1,2,3-triazol-1-yl)methyl)-6-fluoro-7-(3-(2-hydroxyacetyl)-3-azabicyclo[3.1.0]hexan-6-yl)-9,9a-dihydrooxazolo[3,4-a]indol-3(1H)-one
(1R,9aS)-6-fluoro-7-(3-(2-hydroxyacetyl)-3-azabicyclo[3.1.0]hexan-6-yl)-1-((isoxazol-3-yloxy)methyl)-9,9a-dihydrooxazolo[3,4-a]indol-3(1H)-one

Another preferred group of compounds of the present invention is illustrated below.

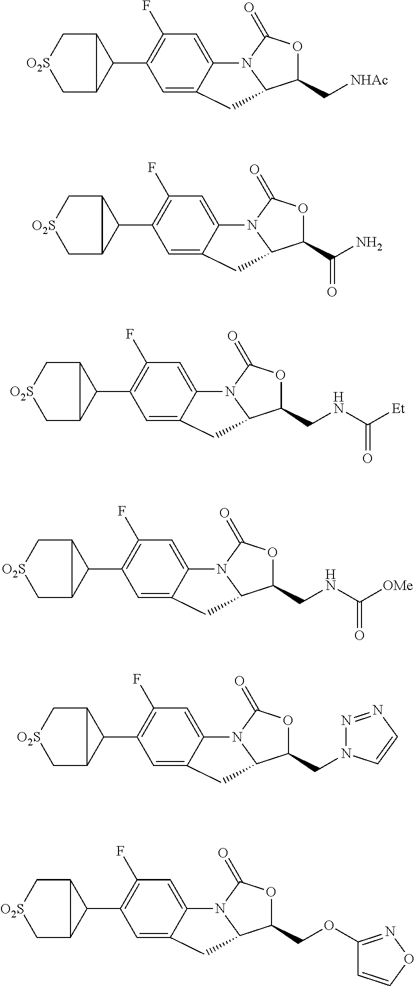

N-(((1S,9aS)-7-(3,3-dioxo-3-thiabicyclo[3.1.0]hexan-6-yl)-6-fluoro-3-oxo-1,3,9,9a-tetrahydrooxazolo[3,4-a]indol-1-yl)methyl)acetamide
(1R,9aS)-7-(3,3-dioxo-3-thiabicyclo[3.1.0]hexan-6-yl)-6-fluoro-3-oxo-1,3,9,9a-tetrahydrooxazolo[3,4-a]indole-1-carboxamide
N-(((1S,9aS)-7-(3,3-dioxo-3-thiabicyclo[3.1.0]hexan-6-yl)-6-fluoro-3-oxo-1,3,9,9a-tetrahydrooxazolo[3,4-a]indol-1-yl)methyl)propionamide
methyl(((1S,9aS)-7-(3,3-dioxo-3-thiabicyclo[3.1.0]hexan-6-yl)-6-fluoro-3-oxo-1,3,9,9a-tetrahydrooxazolo[3,4-a]indol-1-yl)methyl)carbamate
(1S,9aS)-1-((1H-1,2,3-triazol-1-yl)methyl)-7-(3-thiabicyclo[3.1.0]hexan-6-yl)-6-fluoro-9,9a-dihydrooxazolo[3,4-a]indol-3(1H)-one
(1R,9aS)-7-(3,3-dioxo-3-thiabicyclo[3.1.0]hexan-6-yl)-6-fluoro-1-((isoxazol-3-yloxy)methyl)-9,9a-dihydrooxazolo[3,4-a]indol-3(1H)-one

Another preferred group of compounds of the present invention is illustrated below.

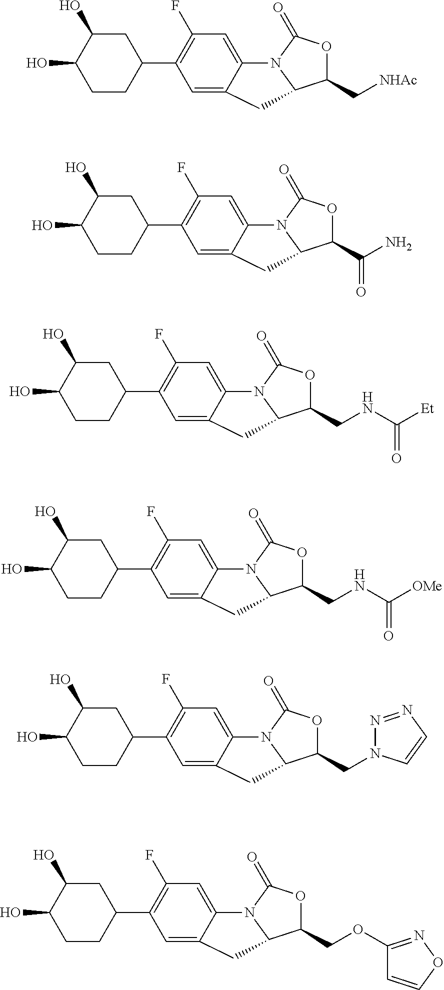

N-(((1S,9aS)-7-((3S,4R)-3,4-dihydroxycyclohexyl)-6-fluoro-3-oxo-1,3,9,9a-tetrahydrooxazolo[3,4-a]indol-1-yl)methyl)acetamide
(1R,9aS)-7-((3S,4R)-3,4-dihydroxycyclohexyl)-6-fluoro-3-oxo-1,3,9,9a-tetrahydrooxazolo[3,4-a]indole-1-carboxamide
N-(((1S,9aS)-7-((3S,4R)-3,4-dihydroxycyclohexyl)-6-fluoro-3-oxo-1,3,9,9a-tetrahydrooxazolo[3,4-a]indol-1-yl)methyl)propionamide
methyl(((1S,9aS)-7-((3S,4R)-3,4-dihydroxycyclohexyl)-6-fluoro-3-oxo-1,3,9,9a-tetrahydrooxazolo[3,4-a]indol-1-yl)methyl)carbamate
(1S,9aS)-1-((1H-1,2,3-triazol-1-yl)methyl)-7-((3S,4R)-3,4-dihydroxycyclohexyl)-6-fluoro-9,9a-dihydrooxazolo[3,4-a]indol-3(1H)-one
(1R,9aS)-7-((3S,4R)-3,4-dihydroxycyclohexyl)-6-fluoro-1-((isoxazol-3-yloxy)methyl)-9,9a-dihydrooxazolo[3,4-a]indol-3(1H)-one

Another preferred group of compounds of the present invention is illustrated below.

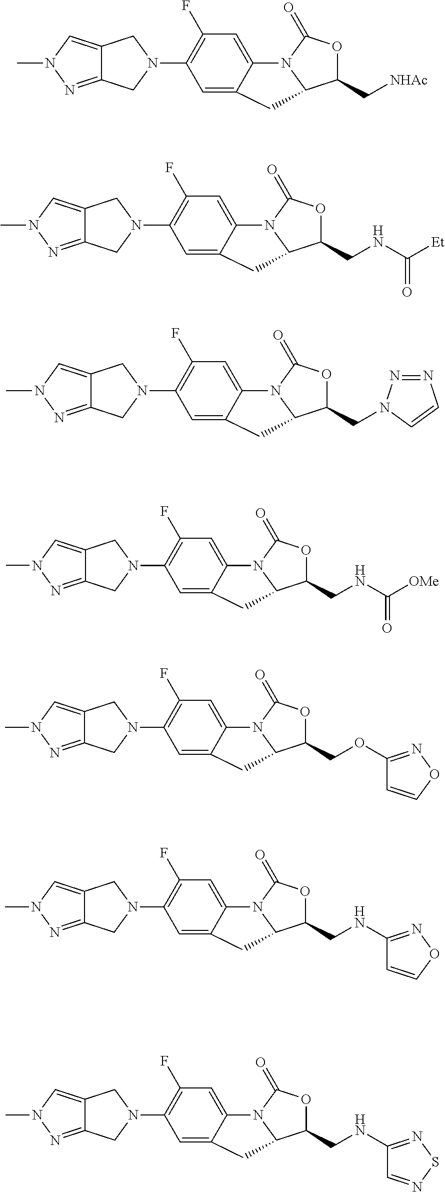

N-(((1S,9aS)-6-fluoro-7-(2-methylpyrrolo[3,4-c]pyrazol-5(2H,4H,6H)-yl)-3-oxo-1,3,9,9a-tetrahydrooxazolo[3,4-a]indol-1-yl)methyl)acetamide
N-(((1S,9aS)-6-fluoro-7-(2-methylpyrrolo[3,4-c]pyrazol-5(2H,4H,6H)-yl)-3-oxo-1,3,9,9a-tetrahydrooxazolo[3,4-a]indol-1-yl)methyl)propionamide
methyl(((1S,9aS)-6-fluoro-7-(2-methylpyrrolo[3,4-c]pyrazol-5(2H,4H,6H)-yl)-3-oxo-1,3,9,9a-tetrahydrooxazolo[3,4-a]indol-1-yl)methyl)carbamate
(1S,9aS)-1-((1H-1,2,3-triazol-1-yl)methyl)-6-fluoro-7-(2-methylpyrrolo[3,4-c]pyrazol-5(2H,4H,6H)-yl)-9,9a-dihydrooxazolo[3,4-a]indol-3(1H)-one
(1S,9aS)-6-fluoro-1-((isoxazol-3-ylamino)methyl)-7-(2-methylpyrrolo[3,4-c]pyrazol-5(2H,4H,6H)-yl)-9,9a-dihydrooxazolo[3,4-a]indol-3(1H)-one
(1R,9aS)-6-fluoro-1-((isoxazol-3-yloxy)methyl)-7-(2-methylpyrrolo[3,4-c]pyrazol-5(2H,4H,6H)-yl)-9,9a-dihydrooxazolo[3,4-a]indol-3(1H)-one
(1R,9aS)-1-((1,2,5-thiadiazol-3-yloxy)methyl)-6-fluoro-7-(2-methylpyrrolo[3,4-c]pyrazol-5(2H,4H,6H)-yl)-9,9a-dihydrooxazolo[3,4-a]indol-3(1H)-one

Another preferred group of compounds of the present invention is illustrated below.

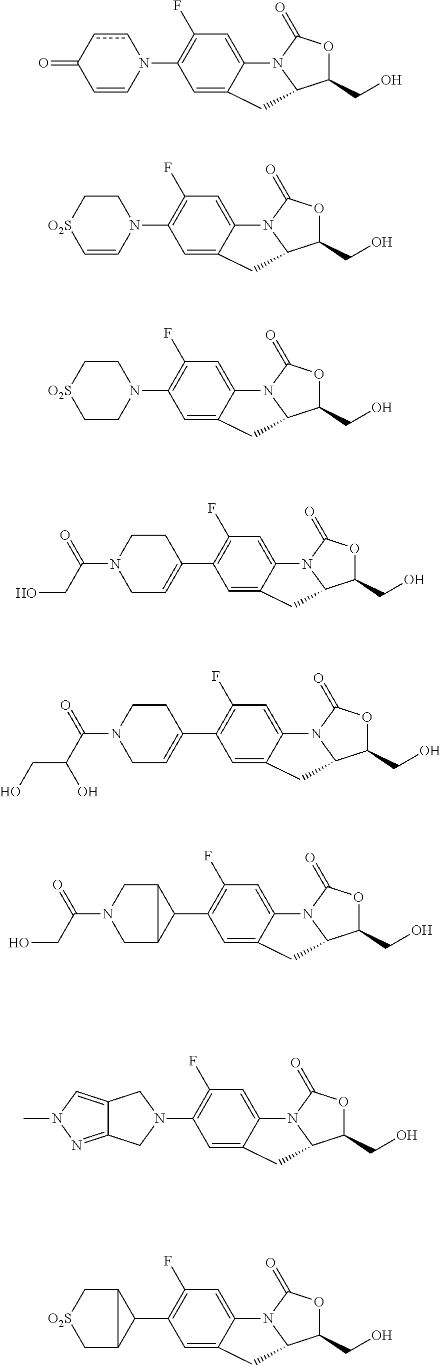

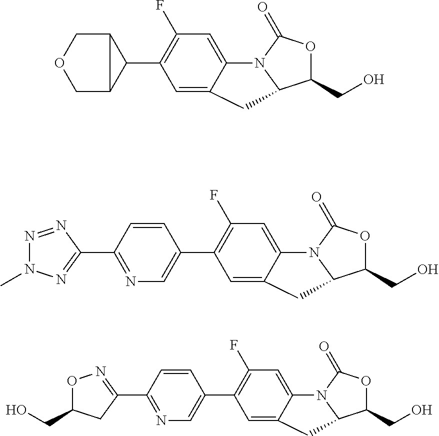

(1R,9aS)-6-fluoro-1-(hydroxymethyl)-7-(4-oxopyridin-1(4H)-yl)-9,9a-dihydrooxazolo[3,4-a]indol-3(1H)-one
(1R,9aS)-6-fluoro-1-(hydroxymethyl)-7-(4-oxo-3,4-dihydropyridin-1(2H)-yl)-9,9a-dihydrooxazolo[3,4-a]indol-3(1H)-one
(1R,9aS)-6-fluoro-1-(hydroxymethyl)-7-(2H-1,1-dioxo-1,4-thiazin-4(3H)-yl)-9,9a-dihydrooxazolo[3,4-a]indol-3(1H)-one
(1R,9aS)-6-fluoro-1-(hydroxymethyl)-7-(1,1-dioxo-2,3,5,6-tetrahydro-1,4-thiazin-4-yl)-9,9a-dihydrooxazolo[3,4-a]indol-3(1H)-one
(1R,9aS)-6-fluoro-7-(1-(2-hydroxyacetyl)-1,2,3,6-tetrahydropyridin-4-yl)-1-(hydroxymethyl)-9,9a-dihydrooxazolo[3,4-a]indol-3(1H)-one
(1R,9aS)-7-(1-(2,3-dihydroxypropanoyl)-1,2,3,6-tetrahydropyridin-4-yl)-6-fluoro-1-(hydroxymethyl)-9,9a-dihydrooxazolo[3,4-a]indol-3(1H)-one
(1R,9aS)-6-fluoro-7-(3-(2-hydroxyacetyl)-3-azabicyclo[3.1.0]hexan-6-yl)-1-(hydroxymethyl)-9,9a-dihydrooxazolo[3,4-a]indol-3(1H)-one
(1R,9aS)-6-fluoro-1-(hydroxymethyl)-7-(2-methylpyrrolo[3,4-c]pyrazol-5(2H,4H,6H)-yl)-9,9a-dihydrooxazolo[3,4-a]indol-3(1H)-one
(1R,9aS)-7-(1,1-dioxo-3-thiabicyclo[3.1.0]hexan-6-yl)-6-fluoro-1-(hydroxymethyl)-9,9a-dihydrooxazolo[3,4-a]indol-3(1H)-one
(1R,9aS)-7-(3-oxabicyclo[3.1.0]hexan-6-yl)-6-fluoro-1-(hydroxymethyl)-9,9a-dihydrooxazolo[3,4-a]indol-3(1H)-one
(1R,9aS)-6-Fluoro-1-(hydroxymethyl)-7-(6-(2-methyl-2H-tetrazol-5-yl)pyridin-3-yl)-9,9a-dihydrooxazolo[3,4-a]indol-3(1H)-one
(1R,9aS)-6-Fluoro-1-(hydroxymethyl)-7-(6-((S)-5-(hydroxymethyl)-4,5-dihydroisoxazol-3-yl)pyridin-3-yl)-9,9a-dihydrooxazolo[3,4-a]indol-3(1H)-one

Another preferred group of compounds of the present invention is illustrated below.

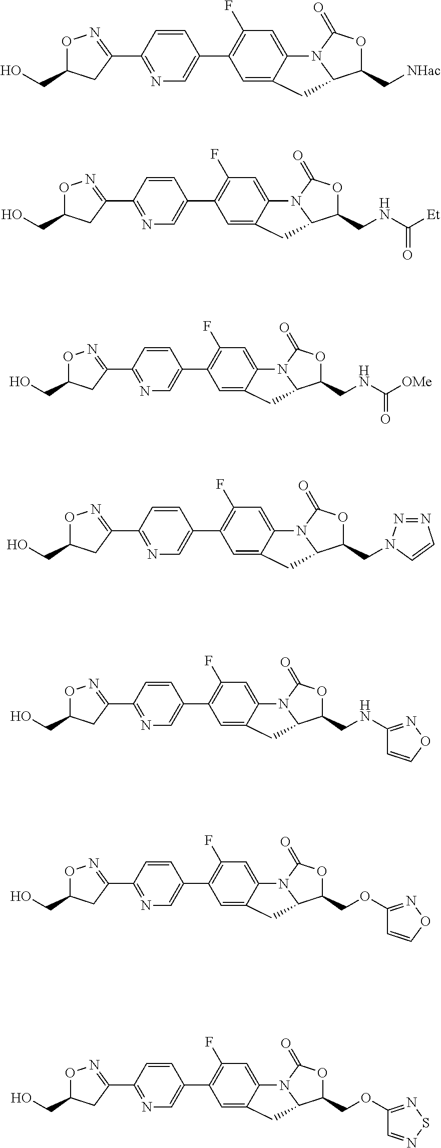

N-(((1S,9aS)-6-fluoro-7-(6-((S)-5-(hydroxymethyl)-4,5-dihydroisoxazol-3-yl)pyridin-3-yl)-3-oxo-1,3,9,9a-tetrahydrooxazolo[3,4-a]indol-1-yl)methyl)acetamide
N-(((1S,9aS)-6-fluoro-7-(6-((S)-5-(hydroxymethyl)-4,5-dihydroisoxazol-3-yl)pyridin-3-yl)-3-oxo-1,3,9,9a-tetrahydrooxazolo[3,4-a]indol-1-yl)methyl)propionamide
methyl(((1S,9aS)-6-fluoro-7-(6-((S)-5-(hydroxymethyl)-4,5-dihydroisoxazol-3-yl)pyridin-3-yl)-3-oxo-1,3,9,9a-tetrahydrooxazolo[3,4-a]indol-1-yl)methyl)carbamate
(1S,9aS)-1-((1H-1,2,3-triazol-1-yl)methyl)-6-fluoro-7-(6-((S)-5-(hydroxymethyl)-4,5-dihydroisoxazol-3-yl)pyridin-3-yl)-9,9a-dihydrooxazolo[3,4-a]indol-3(1H)-one
(1S,9aS)-6-Fluoro-7-(6-((S)-5-(hydroxymethyl)-4,5-dihydroisoxazol-3-yl)pyridin-3-yl)-1-((isoxazol-3-ylamino)methyl)-9,9a-dihydrooxazolo[3,4-a]indol-3(1H)-one
(1R,9aS)-6-fluoro-7-(6-((S)-5-(hydroxymethyl)-4,5-dihydroisoxazol-3-yl)pyridin-3-yl)-1-((isoxazol-3-yloxy)methyl)-9,9a-dihydrooxazolo[3,4-a]indol-3(1H)-one

(1R,9aS)-1-((1,2,5-thiadiazol-3-yloxy)methyl)-6-fluoro-7-(6-((S)-5-(hydroxymethyl)-4,5-dihydroisoxazol-3-yl)pyridin-3-yl)-9,9a-dihydrooxazolo[3,4-a]indol-3(1H)-one

Another preferred group of compounds of the present invention is illustrated below.

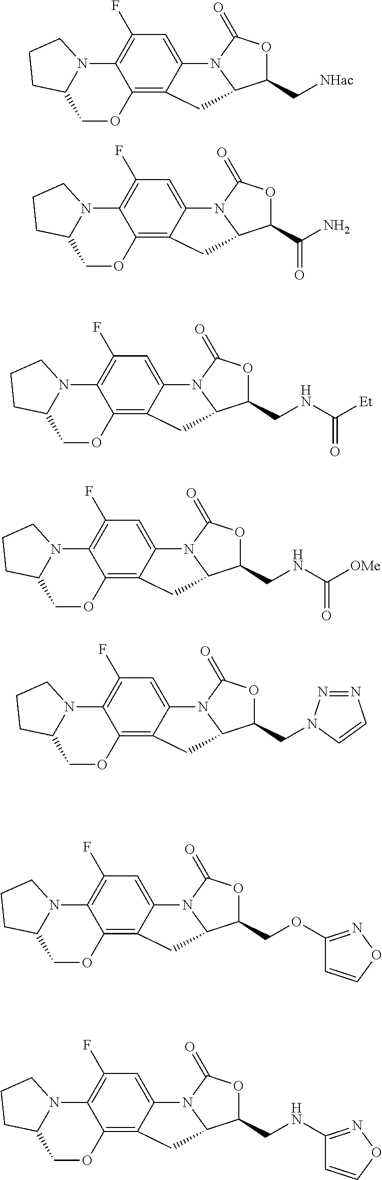

Another preferred group of compounds of the present invention is illustrated below.

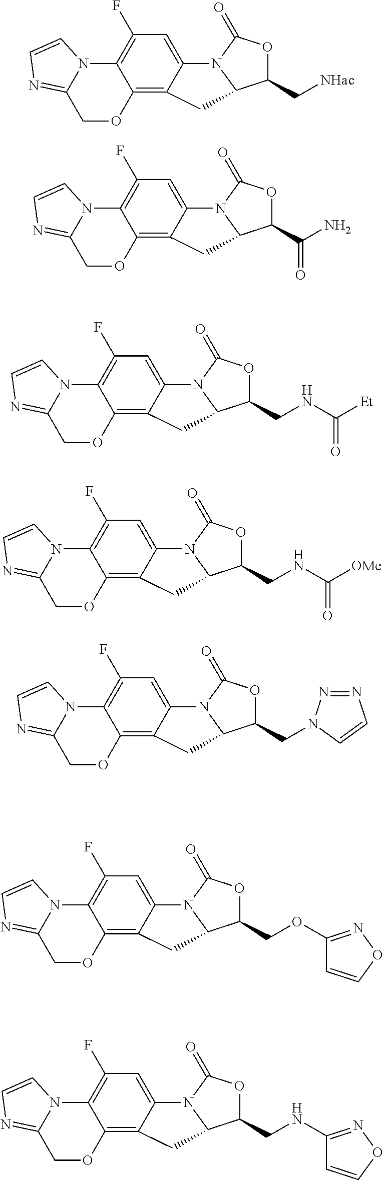

Another preferred group of compounds of the present invention is illustrated below.

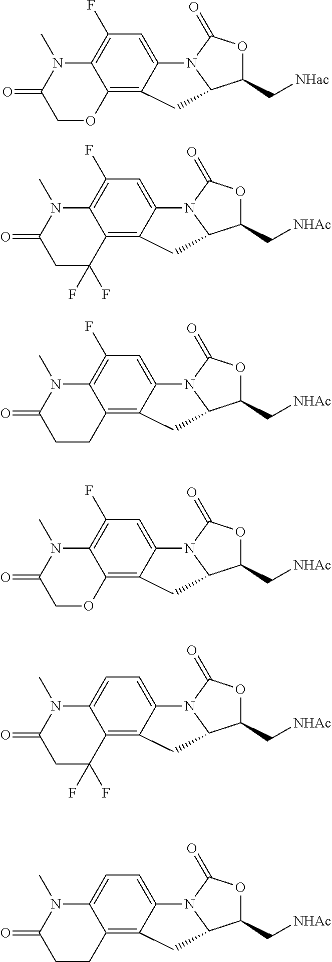

Another preferred group of compounds of the present invention is illustrated below.

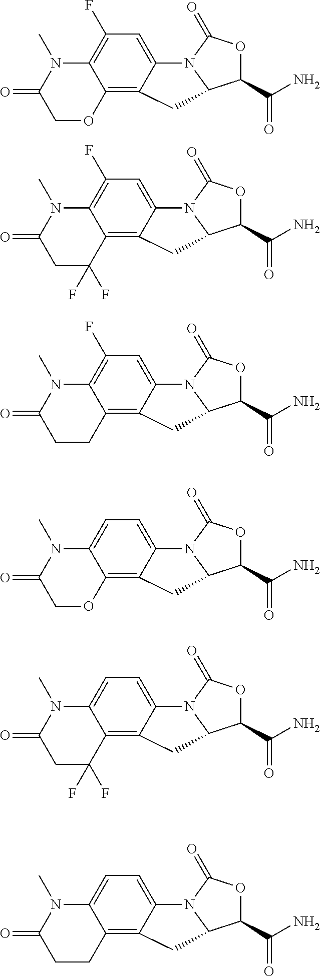

Another preferred group of compounds of the present invention is illustrated below.

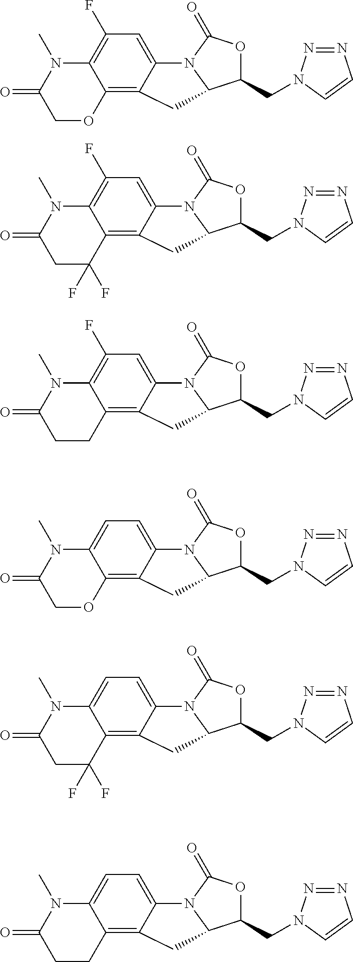

Another preferred group of compounds of the present invention is depicted below.

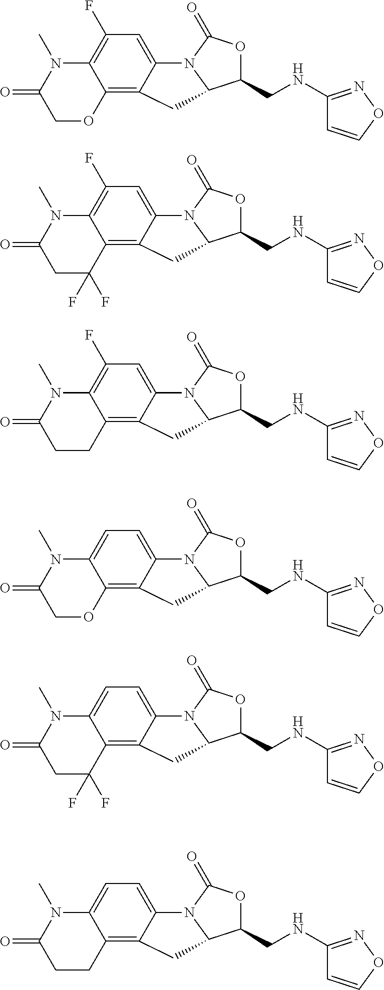

Another preferred group of compounds of the present invention is illustrated below.

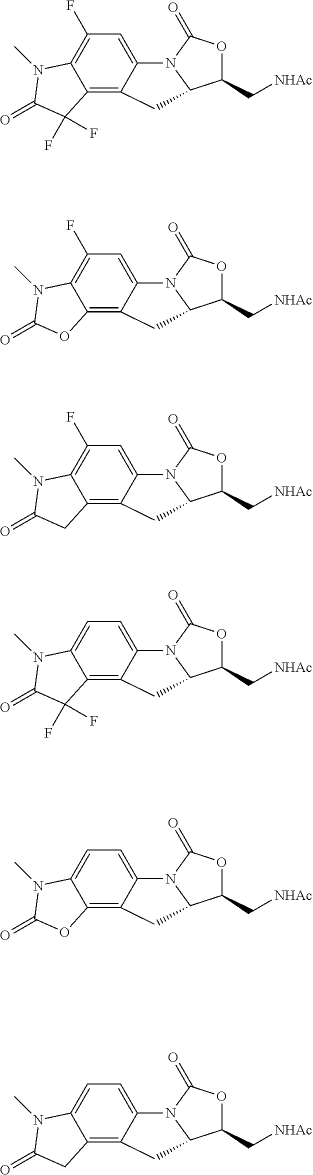

Another preferred group of compounds of the present invention is illustrated below.

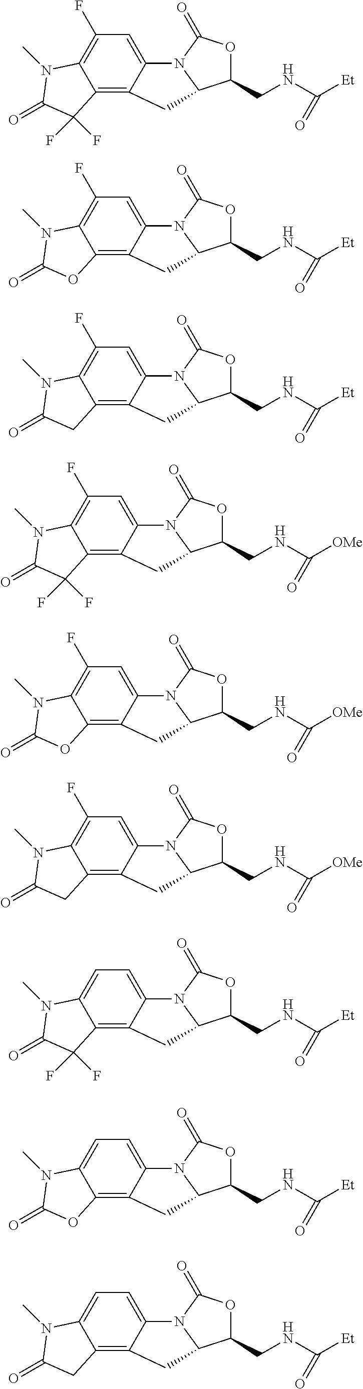

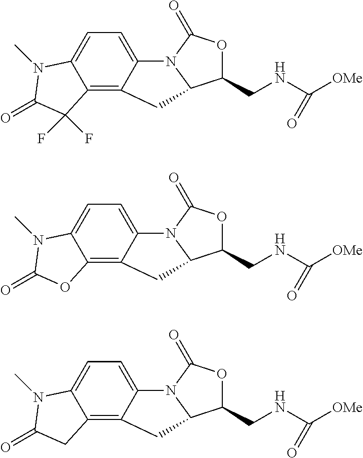

Another preferred group of compounds of the present invention is illustrated below.

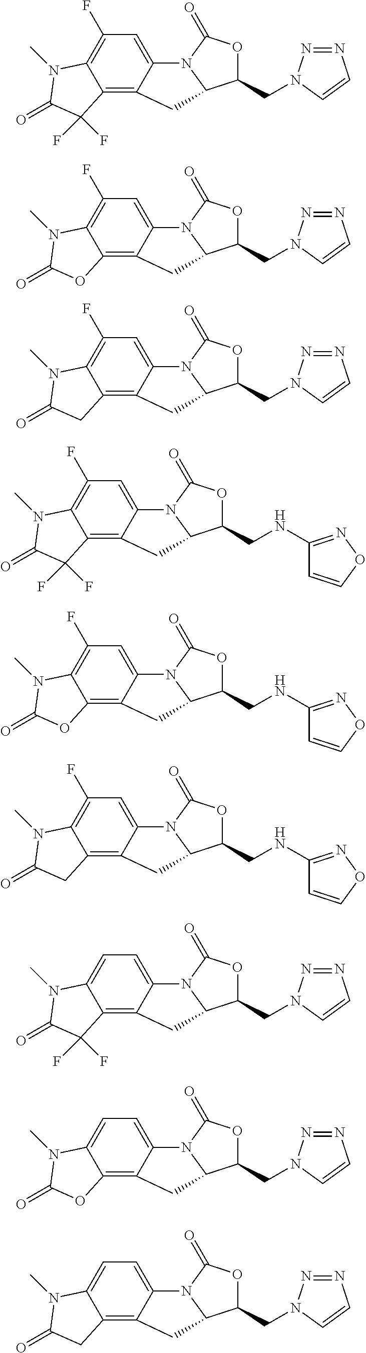

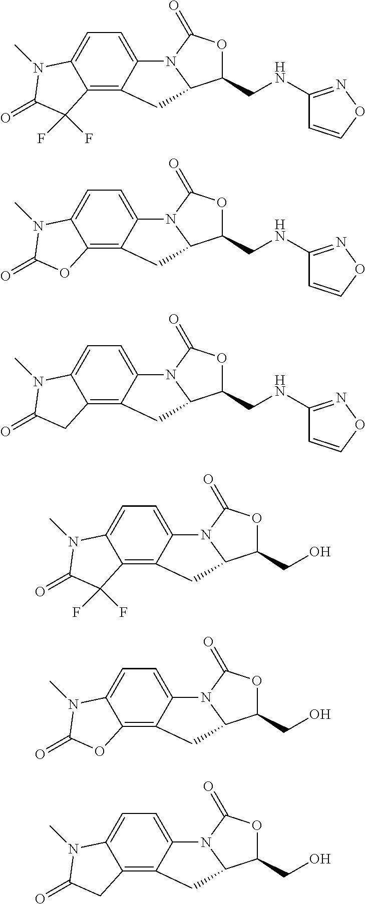

Another preferred group of compounds of the present invention is illustrated below.

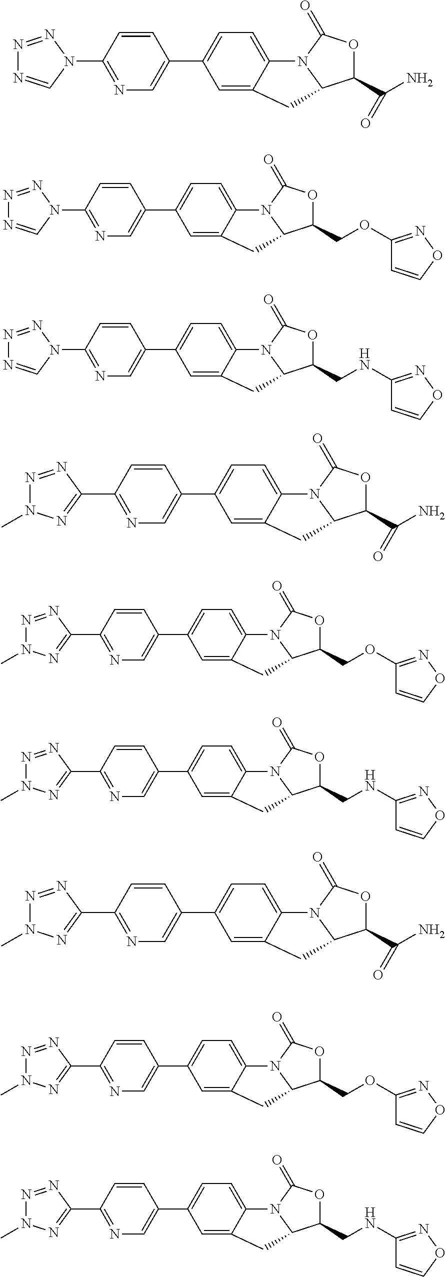

Another preferred group of compounds of the present invention is illustrated below.

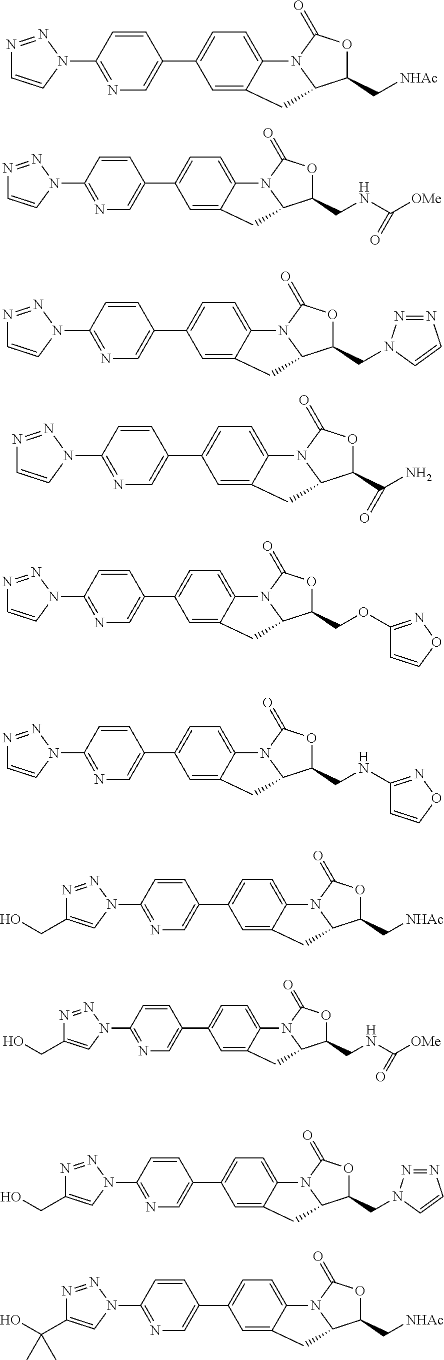

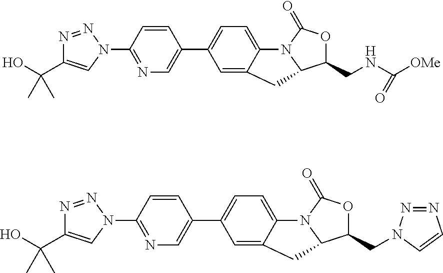

Another preferred group of compounds of the present invention is illustrated below.

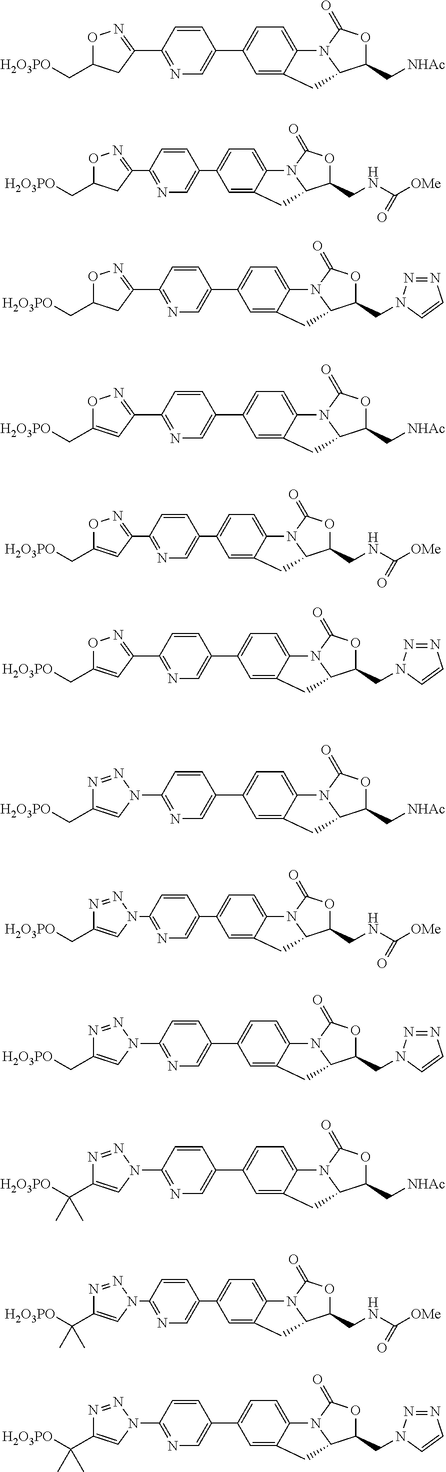

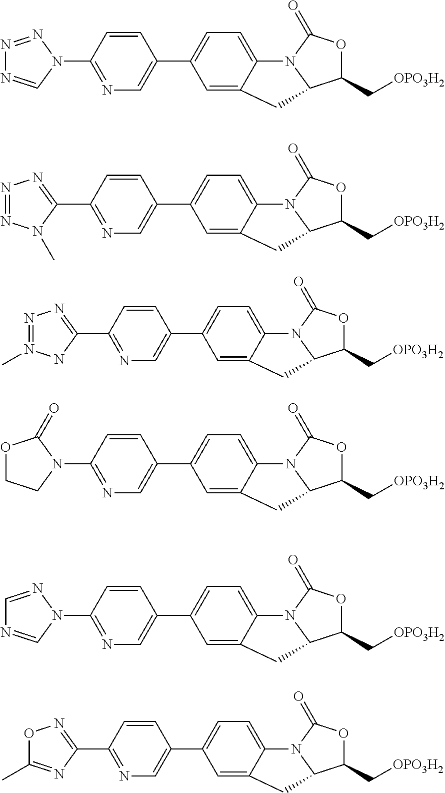

Another preferred group of compounds of the present invention is illustrated below.

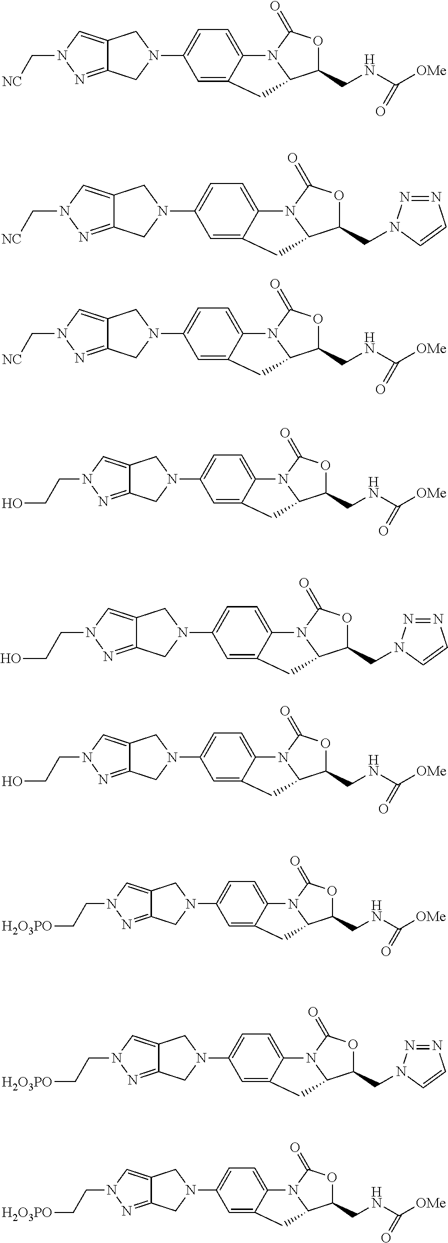

Another preferred group of compounds of the present invention is illustrated below.

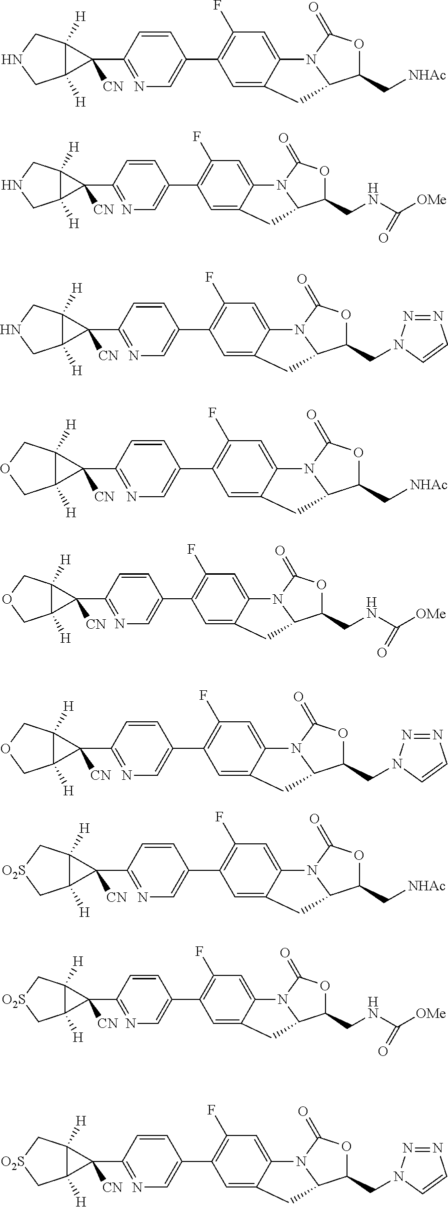

Another preferred group of compounds of the present invention is illustrated below.

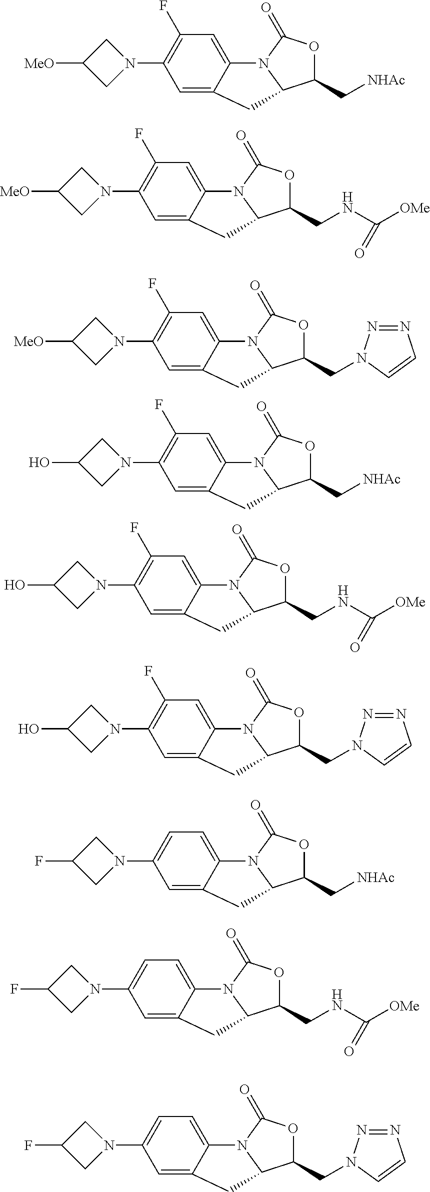

Another preferred group of compounds of the present invention is illustrated below.

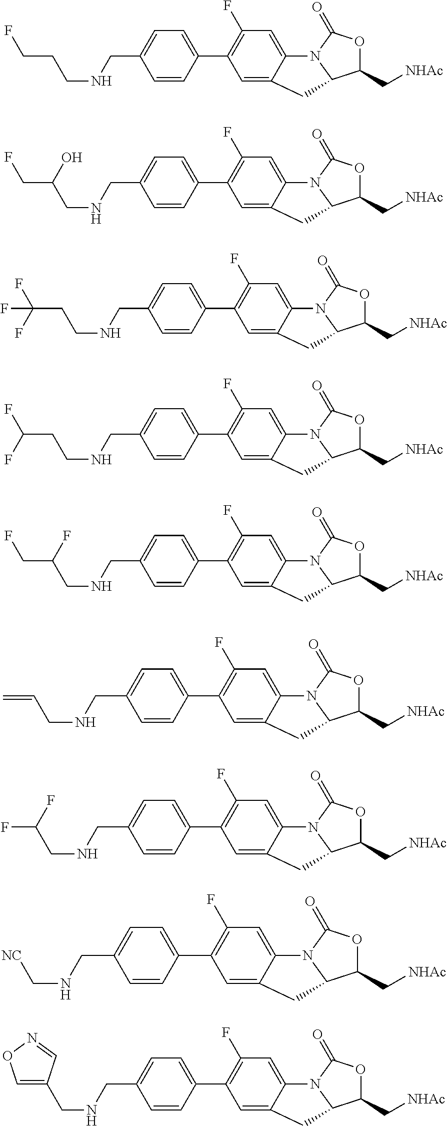

Another preferred group of compounds of the present invention is illustrated below.

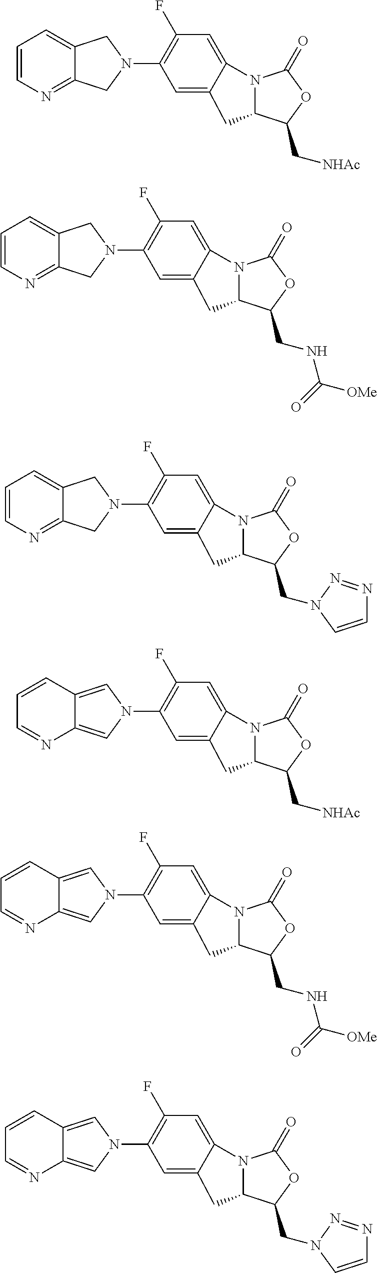

Another preferred group of compounds of the present invention is illustrated below.

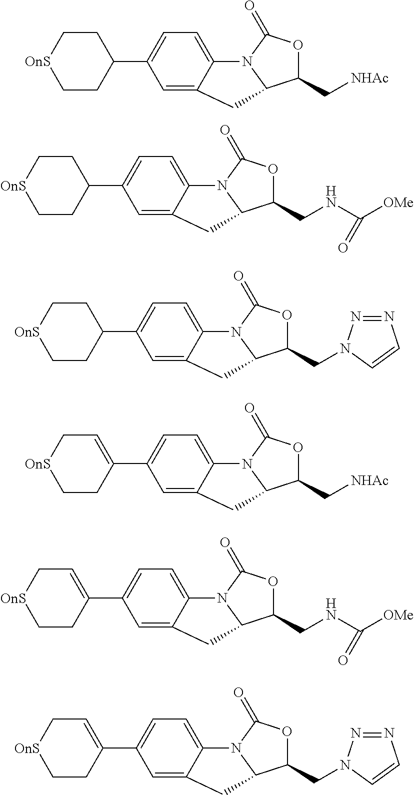

Another preferred group of compounds of the present invention is illustrated below.

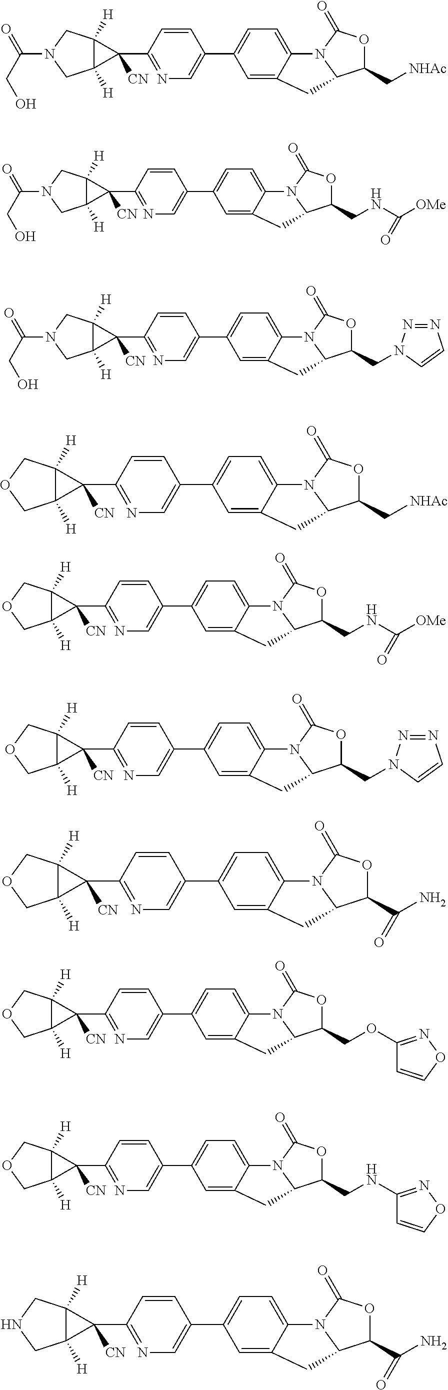

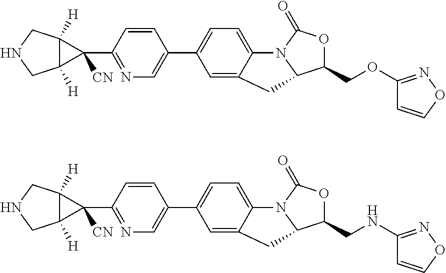

Another preferred group of compounds of the present invention is illustrated below.

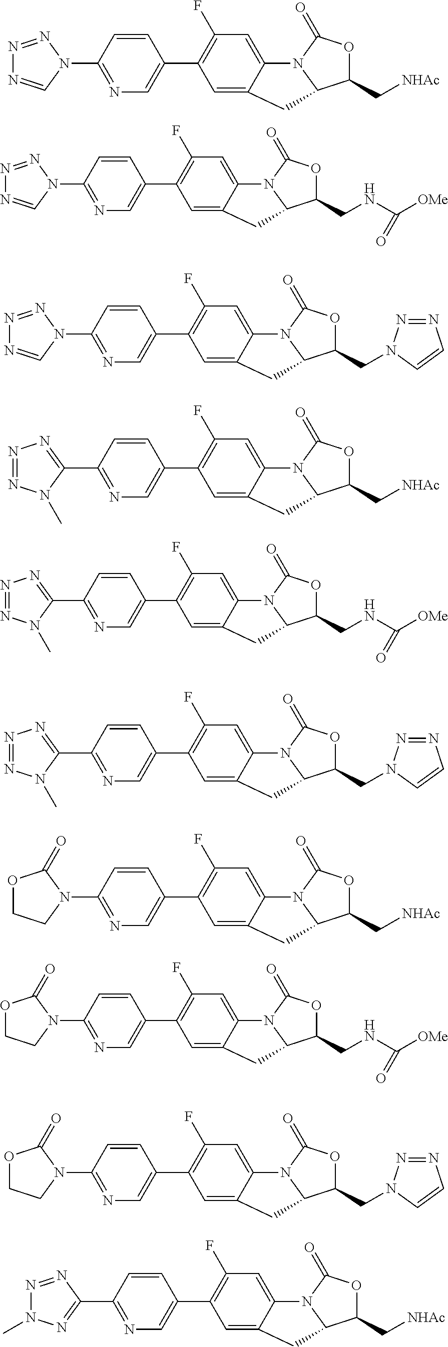

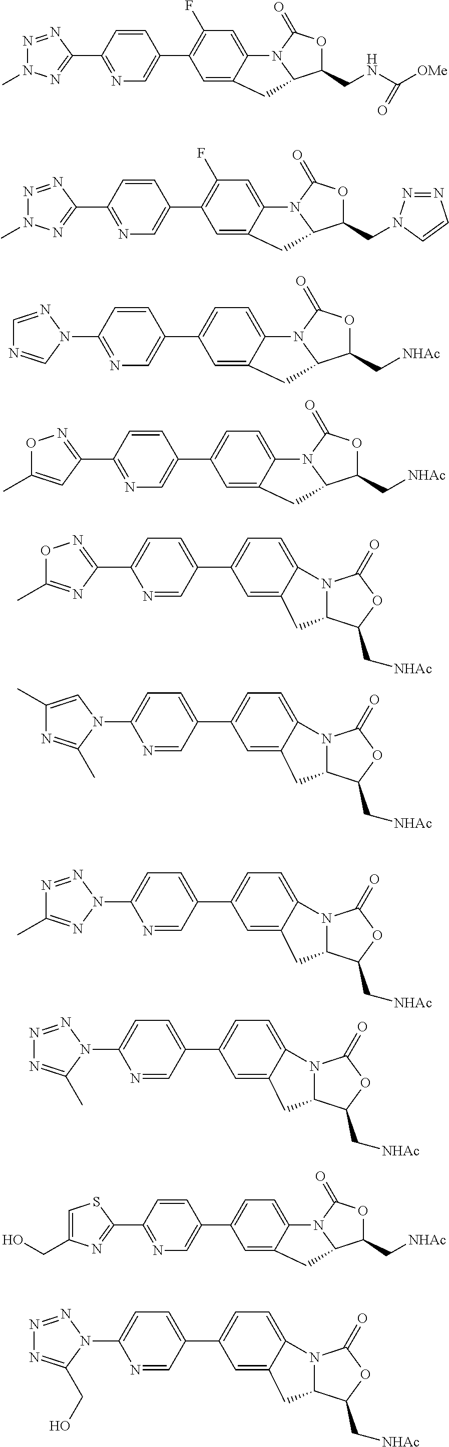

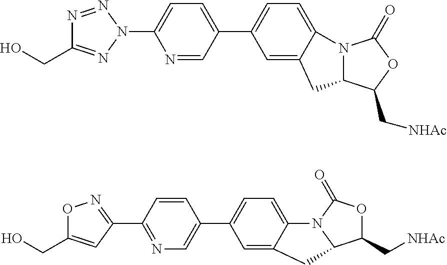

Another preferred group of compounds of the present invention is illustrated below:

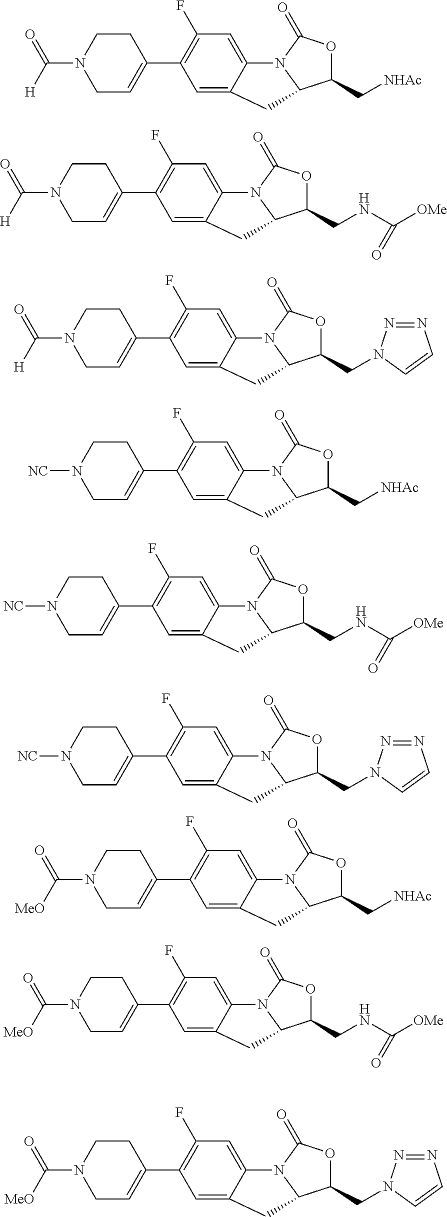

N-(((1S,9aS)-6-fluoro-7-(1-formyl-1,2,3,6-tetrahydropyridin-4-yl)-3-oxo-1,3,9,9a-tetrahydrooxazolo[3,4-a]indol-1-yl)methyl)acetamide;
methyl(((1S,9aS)-6-fluoro-7-(1-formyl-1,2,3,6-tetrahydropyridin-4-yl)-3-oxo-1,3,9,9a-tetrahydrooxazolo[3,4-a]indol-1-yl)methyl)carbamate;
4-((1S,9aS)-1-((1H-1,2,3-triazol-1-yl)methyl)-6-fluoro-3-oxo-1,3,9,9a-tetrahydrooxazolo[3,4-a]indol-7-yl)-5,6-dihydropyridine-1(2H)-carbaldehyde;
N-(((1S,9aS)-7-(1-cyano-1,2,3,6-tetrahydropyridin-4-yl)-6-fluoro-3-oxo-1,3,9,9a-tetrahydrooxazolo[3,4-a]indol-1-yl)methyl)acetamide;
methyl(((1S,9aS)-7-(1-cyano-1,2,3,6-tetrahydropyridin-4-yl)-6-fluoro-3-oxo-1,3,9,9a-tetrahydrooxazolo[3,4-a]indol-1-yl)methyl)carbamate;
4-((1S,9aS)-1-((1H-1,2,3-triazol-1-yl)methyl)-6-fluoro-3-oxo-1,3,9,9a-tetrahydrooxazolo[3,4-a]indol-7-yl)-5,6-dihydropyridine-1(2H)-carbonitrile;
methyl 4-((1S,9aS)-1-(acetamidomethyl)-6-fluoro-3-oxo-1,3,9,9a-tetrahydrooxazolo[3,4-a]indol-7-yl)-5,6-dihydropyridine-1(2H)-carboxylate;
methyl 4-((1S,9aS)-6-fluoro-1-((methoxycarbonylamino)methyl)-3-oxo-1,3,9,9a-tetrahydrooxazolo[3,4-a]indol-7-yl)-5,6-dihydropyridine-1(2H)-carboxylate;
methyl 4-((1S,9aS)-1-((1H-1,2,3-triazol-1-yl)methyl)-6-fluoro-3-oxo-1,3,9,9a-tetrahydrooxazolo[3,4-a]indol-7-yl)-5,6-dihydropyridine-1(2H)-carboxylate.

Another preferred group of compounds of the present invention is illustrated below:

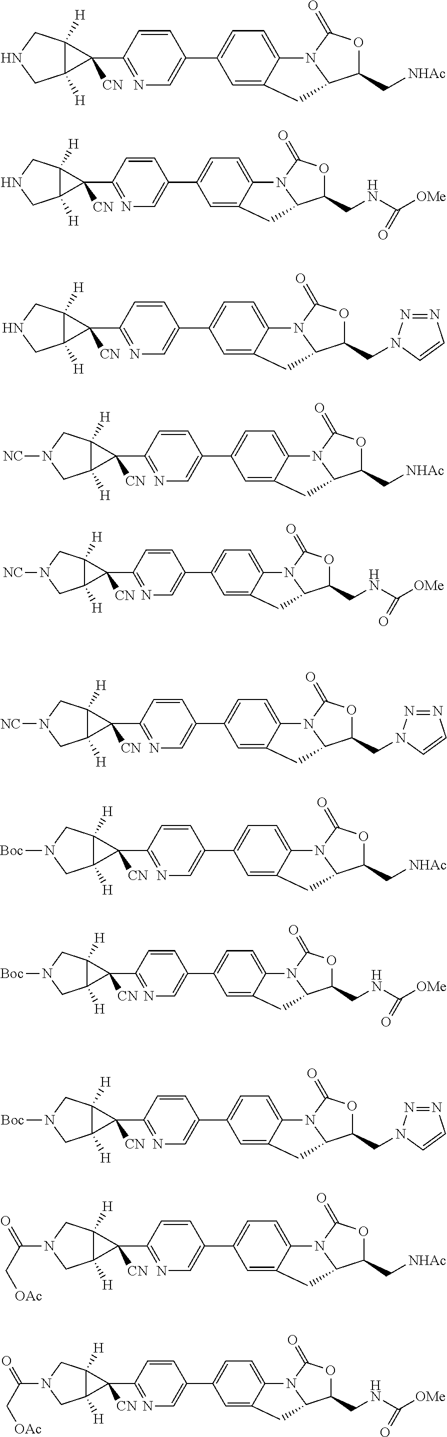

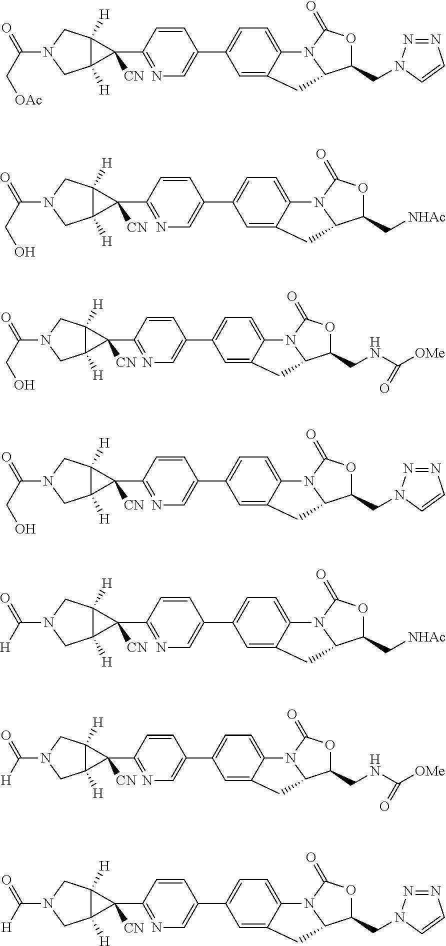

N-(((1S,9aS)-7-(6-((1R,5S,6s)-6-cyano-3-azabicyclo[3.1.0]hexan-6-yl)pyridin-3-yl)-3-oxo-1,3,9,9a-tetrahydrooxazolo[3,4-a]indol-1-yl)methyl)acetamide;
methyl(((1S,9aS)-7-(6-((1R,5S,6s)-6-cyano-3-azabicyclo[3.1.0]hexan-6-yl)pyridin-3-yl)-3-oxo-1,3,9,9a-tetrahydrooxazolo[3,4-a]indol-1-yl)methyl)carbamate;
(1R,5S,6s)-6-(5-((1S,9aS)-1-((1H-1,2,3-triazol-1-yl)methyl)-3-oxo-1,3,9,9a-tetrahydrooxazolo[3,4-a]indol-7-yl)pyridin-2-yl)-3-azabicyclo[3.1.0]hexane-6-carbonitrile;
N-(((1S,9aS)-7-(6-((1R,5S,6s)-3,6-dicyano-3-azabicyclo[3.1.0]hexan-6-yl)pyridin-3-yl)-3-oxo-1,3,9,9a-tetrahydrooxazolo[3,4-a]indol-1-yl)methyl)acetamide;
methyl(((1S,9aS)-7-(6-((1R,5S,6s)-3,6-dicyano-3-azabicyclo[3.1.0]hexan-6-yl)pyridin-3-yl)-3-oxo-1,3,9,9a-tetrahydrooxazolo[3,4-a]indol-1-yl)methyl)carbamate;
(1R,5S,6s)-6-(5-((1S,9aS)-1-((1H-1,2,3-triazol-1-yl)methyl)-3-oxo-1,3,9,9a-tetrahydrooxazolo[3,4-a]indol-7-yl)pyridin-2-yl)-3-azabicyclo[3.1.0]hexane-3,6-dicarbonitrile;
(1R,5S,6s)-tert-butyl 6-(5-((1S,9aS)-1-(acetamidomethyl)-3-oxo-1,3,9,9a-tetrahydrooxazolo[3,4-a]indol-7-yl)pyridin-2-yl)-6-cyano-3-azabicyclo[3.1.0]hexane-3-carboxylate;
(1R,5S,6s)-tert-butyl 6-cyano-6-(5-((1S,9aS)-1-((methoxycarbonylamino)methyl)-3-oxo-1,3,9,9a-tetrahydrooxazolo[3,4-a]indol-7-yl)pyridin-2-yl)-3-azabicyclo[3.1.0]hexane-3-carboxylate;
(1R,5S,6s)-tert-butyl 6-(5-((1S,9aS)-1-((1H-1,2,3-triazol-1-yl)methyl)-3-oxo-1,3,9,9a-tetrahydrooxazolo[3,4-a]indol-7-yl)pyridin-2-yl)-6-cyano-3-azabicyclo[3.1.0]hexane-3-carboxylate;
2-((1R,5S,6s)-6-(5-((1S,9aS)-1-(acetamidomethyl)-3-oxo-1,3,9,9a-tetrahydrooxazolo[3,4-a]indol-7-yl)pyridin-2-yl)-6-cyano-3-azabicyclo[3.1.0]hexan-3-yl)-2-oxoethyl acetate;
2-((1R,5S,6s)-6-cyano-6-(5-((1S,9aS)-1-((methoxycarbonylamino)methyl)-3-oxo-1,3,9,9a-tetrahydrooxazolo[3,4-a]indol-7-yl)pyridin-2-yl)-3-azabicyclo[3.1.0]hexan-3-yl)-2-oxoethyl acetate;
2-((1R,5S,6s)-6-(5-((1S,9aS)-1-((1H-1,2,3-triazol-1-yl)methyl)-3-oxo-1,3,9,9a-tetrahydrooxazolo[3,4-a]indol-7-yl)pyridin-2-yl)-6-cyano-3-azabicyclo[3.1.0]hexan-3-yl)-2-oxoethyl acetate;
N-(((1S,9aS)-7-(6-((1R,5S,6s)-6-cyano-3-(2-hydroxyacetyl)-3-azabicyclo[3.1.0]hexan-6-yl)pyridin-3-yl)-3-oxo-1,3,9,9a-tetrahydrooxazolo[3,4-a]indol-1-yl)methyl)acetamide;
methyl(((1S,9aS)-7-(6-((1R,5S,6s)-6-cyano-3-(2-hydroxyacetyl)-3-azabicyclo[3.1.0]hexan-6-yl)pyridin-3-yl)-3-oxo-1,3,9,9a-tetrahydrooxazolo[3,4-a]indol-1-yl)methyl)carbamate;
(1R,5S,6s)-6-(5-((1S,9aS)-1-((1H-1,2,3-triazol-1-yl)methyl)-3-oxo-1,3,9,9a-tetrahydrooxazolo[3,4-a]indol-7-yl)pyridin-2-yl)-3-(2-hydroxyacetyl)-3-azabicyclo[3.1.0]hexane-6-carbonitrile;
N-(((1S,9aS)-7-(6-((1R,5S,6s)-6-cyano-3-formyl-3-azabicyclo[3.1.0]hexan-6-yl)pyridin-3-yl)-3-oxo-1,3,9,9a-tetrahydrooxazolo[3,4-a]indol-1-yl)methyl)acetamide;
methyl(((1S,9aS)-7-(6-((1R,5S,6s)-6-cyano-3-formyl-3-azabicyclo[3.1.0]hexan-6-yl)pyridin-3-yl)-3-oxo-1,3,9,9a-tetrahydrooxazolo[3,4-a]indol-1-yl)methyl)carbamate;
(1R,5S,6s)-6-(5-((1S,9aS)-1-((1H-1,2,3-triazol-1-yl)methyl)-3-oxo-1,3,9,9a-tetrahydrooxazolo[3,4-a]indol-7-yl)pyridin-2-yl)-3-formyl-3-azabicyclo[3.1.0]hexane-6-carbonitrile.

Another preferred group of compounds of the present invention is illustrated below:

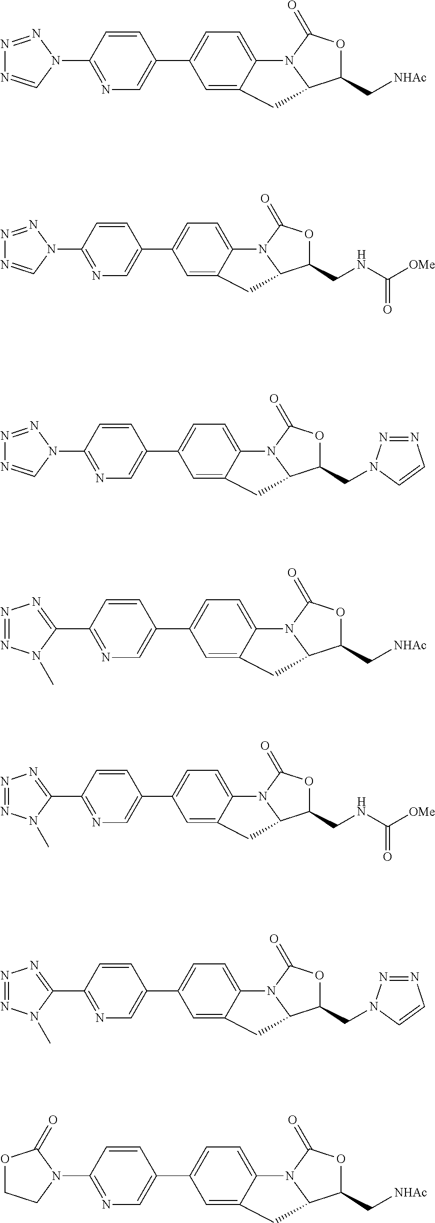

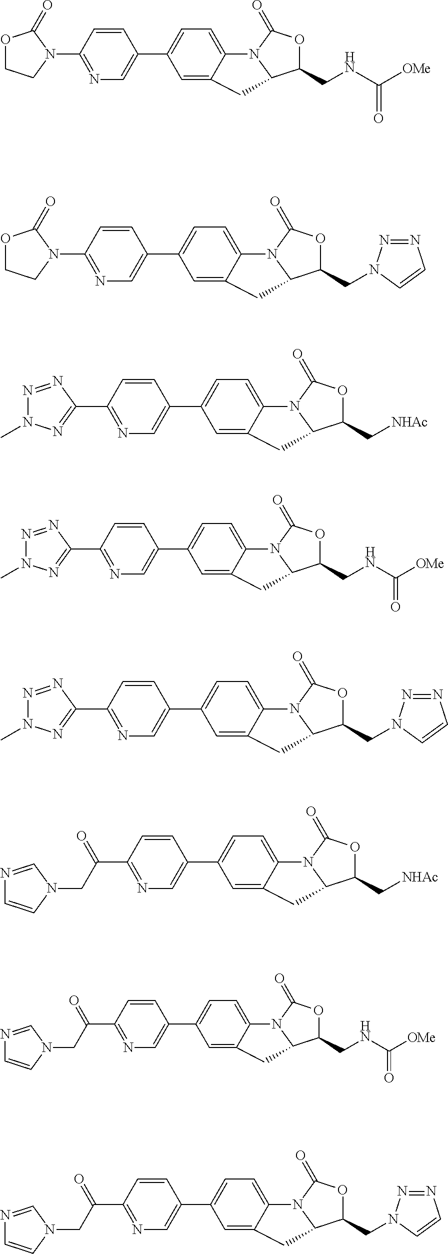

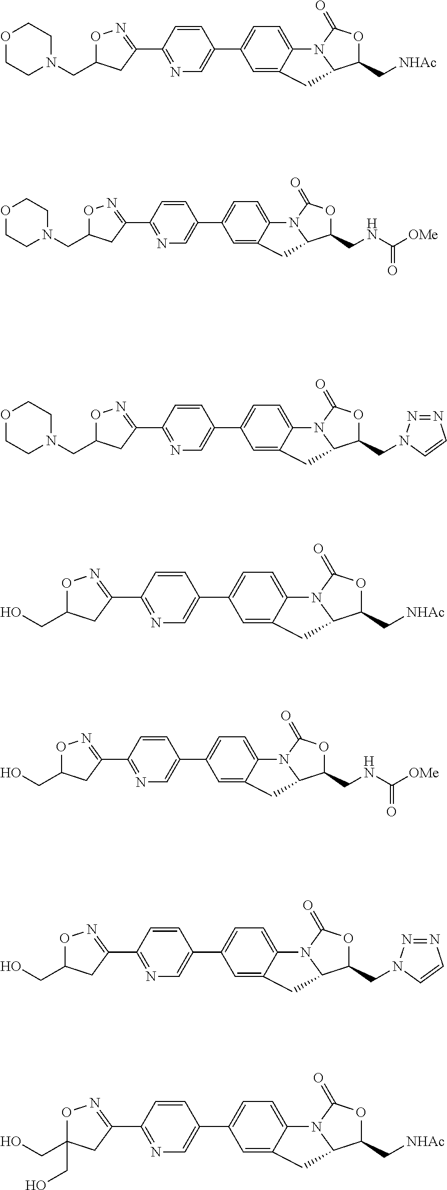

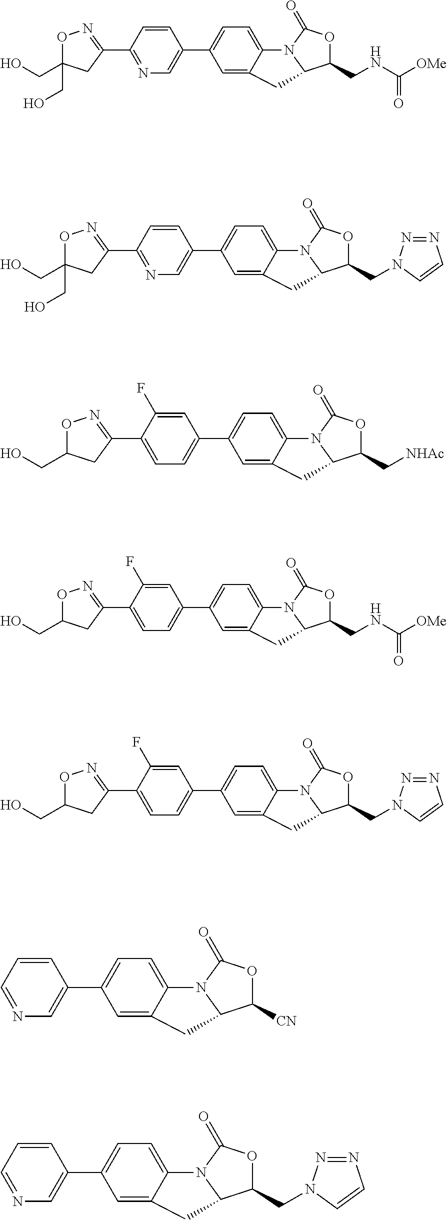

N-(((1S,9aS)-7-(6-(1H-tetrazol-1-yl)pyridin-3-yl)-3-oxo-1,3,9,9a-tetrahydrooxazolo[3,4-a]indol-1-yl)methyl)acetamide;
methyl(((1S,9aS)-7-(6-(1H-tetrazol-1-yl)pyridin-3-yl)-3-oxo-1,3,9,9a-tetrahydrooxazolo[3,4-a]indol-1-yl)methyl)carbamate;
(1S,9aS)-1-((1H-1,2,3-triazol-1-yl)methyl)-7-(6-(1H-tetrazol-1-yl)pyridin-3-yl)-9,9a-dihydrooxazolo[3,4-a]indol-3 (1H)-one;
N-(((1S,9aS)-7-(6-(1-methyl-1H-tetrazol-5-yl)pyridin-3-yl)-3-oxo-1,3,9,9a-tetrahydrooxazolo[3,4-a]indol-1-yl)methyl)acetamide;
methyl(((1S,9aS)-7-(6-(1-methyl-1H-tetrazol-5-yl)pyridin-3-yl)-3-oxo-1,3,9,9a-tetrahydrooxazolo[3,4-a]indol-1-yl)methyl)carbamate;
(1S,9aS)-1-((1H-1,2,3-triazol-1-yl)methyl)-7-(6-(1-methyl-1H-tetrazol-5-yl)pyridin-3-yl)-9,9a-dihydrooxazolo[3,4-a]indol-3 (1H)-one;
N-(((1S,9aS)-3-oxo-7-(6-(2-oxooxazolidin-3-yl)pyridin-3-yl)-1,3,9,9a-tetrahydrooxazolo[3,4-a]indol-1-yl)methyl)acetamide;
methyl(((1S,9aS)-3-oxo-7-(6-(2-oxooxazolidin-3-yl)pyridin-3-yl)-1,3,9,9a-tetrahydrooxazolo[3,4-a]indol-1-yl)methyl)carbamate;
(1S,9aS)-1-((1H-1,2,3-triazol-1-yl)methyl)-7-(6-(2-oxooxazolidin-3-yl)pyridin-3-yl)-9,9a-dihydrooxazolo[3,4-a]indol-3 (1H)-one;
N-(((1S,9aS)-7-(6-(2-methyl-2H-tetrazol-5-yl)pyridin-3-yl)-3-oxo-1,3,9,9a-tetrahydrooxazolo[3,4-a]indol-1-yl)methyl)acetamide;
methyl(((1S,9aS)-7-(6-(2-methyl-2H-tetrazol-5-yl)pyridin-3-yl)-3-oxo-1,3,9,9a-tetrahydrooxazolo[3,4-a]indol-1-yl)methyl)carbamate;
(1S,9aS)-1-((1H-1,2,3-triazol-1-yl)methyl)-7-(6-(2-methyl-2H-tetrazol-5-yl)pyridin-3-yl)-9,9a-dihydrooxazolo[3,4-a]indol-3 (1H)-one;
N-(((1S,9aS)-7-(6-(2-(1H-imidazol-1-yl)acetyl)pyridin-3-yl)-3-oxo-1,3,9,9a-tetrahydrooxazolo[3,4-a]indol-1-yl)methyl)acetamide;
methyl(((1S,9aS)-7-(6-(2-(1H-imidazol-1-yl)acetyl)pyridin-3-yl)-3-oxo-1,3,9,9a-tetrahydrooxazolo[3,4-a]indol-1-yl)methyl)carbamate;
(1S,9aS)-1-((1H-1,2,3-triazol-1-yl)methyl)-7-(6-(2-(1H-imidazol-1-yl)acetyl)pyridin-3-yl)-9,9a-dihydrooxazolo[3,4-a]indol-3 (1H)-one;
N-(((1S,9aS)-7-(6-(5-(morpholinomethyl)-4,5-dihydroisoxazol-3-yl)pyridin-3-yl)-3-oxo-1,3,9,9a-tetrahydrooxazolo[3,4-a]indol-1-yl)methyl)acetamide;
methyl(((1S,9aS)-7-(6-(5-(morpholinomethyl)-4,5-dihydroisoxazol-3-yl)pyridin-3-yl)-3-oxo-1,3,9,9a-tetrahydrooxazolo[3,4-a]indol-1-yl)methyl)carbamate;
(1S,9aS)-1-((1H-1,2,3-triazol-1-yl)methyl)-7-(6-(5-(morpholinomethyl)-4,5-dihydroisoxazol-3-yl)pyridin-3-yl)-9,9a-dihydrooxazolo[3,4-a]indol-3 (1H)-one;
N-(((1S,9aS)-7-(6-(5-(hydroxymethyl)-4,5-dihydroisoxazol-3-yl)pyridin-3-yl)-3-oxo-1,3,9,9a-tetrahydrooxazolo[3,4-a]indol-1-yl)methyl)acetamide;
methyl(((1S,9aS)-7-(6-(5-(hydroxymethyl)-4,5-dihydroisoxazol-3-yl)pyridin-3-yl)-3-oxo-1,3,9,9a-tetrahydrooxazolo[3,4-a]indol-1-yl)methyl)carbamate;
(1S,9aS)-1-((1H-1,2,3-triazol-1-yl)methyl)-7-(6-(5-(hydroxymethyl)-4,5-dihydroisoxazol-3-yl)pyridin-3-yl)-9,9a-dihydrooxazolo[3,4-a]indol-3 (1H)-one;
N-(((1S,9aS)-7-(6-(5,5-bis(hydroxymethyl)-4,5-dihydroisoxazol-3-yl)pyridin-3-yl)-3-oxo-1,3,9,9a-tetrahydrooxazolo[3,4-a]indol-1-yl)methyl)acetamide;
methyl(((1S,9aS)-7-(6-(5,5-bis(hydroxymethyl)-4,5-dihydroisoxazol-3-yl)pyridin-3-yl)-3-oxo-1,3,9,9a-tetrahydrooxazolo[3,4-a]indol-1-yl)methyl)carbamate;
(1S,9aS)-1-((1H-1,2,3-triazol-1-yl)methyl)-7-(6-(5,5-bis(hydroxymethyl)-4,5-dihydroisoxazol-3-yl)pyridin-3-yl)-9,9a-dihydrooxazolo[3,4-a]indol-3(1H)-one;
N-(((1S,9aS)-7-(3-fluoro-4-(5-(hydroxymethyl)-4,5-dihydroisoxazol-3-yl)phenyl)-3-oxo-1,3,9,9a-tetrahydrooxazolo[3,4-a]indol-1-yl)methyl)acetamide;
methyl(((1S,9aS)-7-(3-fluoro-4-(5-(hydroxymethyl)-4,5-dihydroisoxazol-3-yl)phenyl)-3-oxo-1,3,9,9a-tetrahydrooxazolo[3,4-a]indol-1-yl)methyl)carbamate;
(1S,9aS)-1-((1H-1,2,3-triazol-1-yl)methyl)-7-(3-fluoro-4-(5-(hydroxymethyl)-4,5-dihydroisoxazol-3-yl)phenyl)-9,9a-dihydrooxazolo[3,4-a]indol-3 (1H)-one;
(1S,9aS)-1-((1H-1,2,3-triazol-1-yl)methyl)-7-(pyridin-3-yl)-9,9a-dihydrooxazolo[3,4-a]indol-3(1H)-one;
(1R,9aS)-3-oxo-7-(pyridin-3-yl)-1,3,9,9a-tetrahydrooxazolo[3,4-a]indole-1-carbonitrile.

Another preferred group of compounds of the present invention is illustrated below:
N-(((1S,9aS)-7-(6-(1H-1,2,3-triazol-1-yl)pyridin-3-yl)-3-oxo-1,3,9,9a-tetrahydrooxazolo[3,4-a]indol-1-yl)methyl)acetamide;
methyl(((1S,9aS)-7-(6-(1H-1,2,3-triazol-1-yl)pyridin-3-yl)-3-oxo-1,3,9,9a-tetrahydrooxazolo[3,4-a]indol-1-yl)methyl)carbamate;
(1S,9aS)-1-((1H-1,2,3-triazol-1-yl)methyl)-7-(6-(1H-1,2,3-triazol-1-yl)pyridin-3-yl)-9,9a-dihydrooxazolo[3,4-a]indol-3 (1H)-one;
(1R,9aS)-7-(6-(1H-1,2,3-triazol-1-yl)pyridin-3-yl)-3-oxo-1,3,9,9a-tetrahydrooxazolo[3,4-a]indole-1-carboxamide;
(1R,9aS)-7-(6-(1H-1,2,3-triazol-1-yl)pyridin-3-yl)-1-((isoxazol-3-yloxy)methyl)-9,9a-dihydrooxazolo[3,4-a]indol-3 (1H)-one;
(1S,9aS)-7-(6-(1H-1,2,3-triazol-1-yl)pyridin-3-yl)-1-((isoxazol-3-ylamino)methyl)-9,9a-dihydrooxazolo[3,4-a]indol-3(1H)-one;
N-(((1S,9aS)-7-(6-(4-(hydroxymethyl)-1H-1,2,3-triazol-1-yl)pyridin-3-yl)-3-oxo-1,3,9,9a-tetrahydrooxazolo[3,4-a]indol-1-yl)methyl)acetamide;
methyl(((1S,9aS)-7-(6-(4-(hydroxymethyl)-1H-1,2,3-triazol-1-yl)pyridin-3-yl)-3-oxo-1,3,9,9a-tetrahydrooxazolo[3,4-a]indol-1-yl)methyl)carbamate;
(1S,9aS)-1-((1H-1,2,3-triazol-1-yl)methyl)-7-(6-(4-(hydroxymethyl)-1H-1,2,3-triazol-1-yl)pyridin-3-yl)-9,9a-dihydrooxazolo[3,4-a]indol-3(1H)-one;
N-(((1S,9aS)-7-(6-(4-(2-hydroxypropan-2-yl)-1H-1,2,3-triazol-1-yl)pyridin-3-yl)-3-oxo-1,3,9,9a-tetrahydrooxazolo[3,4-a]indol-1-yl)methyl)acetamide;
methyl(((1S,9aS)-7-(6-(4-(2-hydroxypropan-2-yl)-1H-1,2,3-triazol-1-yl)pyridin-3-yl)-3-oxo-1,3,9,9a-tetrahydrooxazolo[3,4-a]indol-1-yl)methyl)carbamate;
(1S,9aS)-1-((1H-1,2,3-triazol-1-yl)methyl)-7-(6-(4-(2-hydroxypropan-2-yl)-1H-1,2,3-triazol-1-yl)pyridin-3-yl)-9,9a-dihydrooxazolo[3,4-a]indol-3(1H)-one.

Another preferred group of compounds of the present invention is illustrated below:
N-(((1S,9aS)-7-(6-(1H-tetrazol-1-yl)pyridin-3-yl)-6-fluoro-3-oxo-1,3,9,9a-tetrahydrooxazolo[3,4-a]indol-1-yl)methyl)acetamide;
methyl(((1S,9aS)-7-(6-(1H-tetrazol-1-yl)pyridin-3-yl)-6-fluoro-3-oxo-1,3,9,9a-tetrahydrooxazolo[3,4-a]indol-1-yl)methyl)carbamate;
(1S,9aS)-1-((1H-1,2,3-triazol-1-yl)methyl)-7-(6-(1H-tetrazol-1-yl)pyridin-3-yl)-6-fluoro-9,9a-dihydrooxazolo[3,4-a]indol-3(1H)-one;
N-(((1S,9aS)-6-fluoro-7-(6-(1-methyl-1H-tetrazol-5-yl)pyridin-3-yl)-3-oxo-1,3,9,9a-tetrahydrooxazolo[3,4-a]indol-1-yl)methyl)acetamide;
methyl(((1S,9aS)-6-fluoro-7-(6-(1-methyl-1H-tetrazol-5-yl)pyridin-3-yl)-3-oxo-1,3,9,9a-tetrahydrooxazolo[3,4-a]indol-1-yl)methyl)carbamate;
(1S,9aS)-1-((1H-1,2,3-triazol-1-yl)methyl)-6-fluoro-7-(6-(1-methyl-1H-tetrazol-5-yl)pyridin-3-yl)-9,9a-dihydrooxazolo[3,4-a]indol-3(1H)-one;
N-(((1S,9aS)-6-fluoro-3-oxo-7-(6-(2-oxooxazolidin-3-yl)pyridin-3-yl)-1,3,9,9a-tetrahydrooxazolo[3,4-a]indol-1-yl)methyl)acetamide;
methyl(((1S,9aS)-7-(5-fluoro-6-(2-oxooxazolidin-3-yl)pyridin-3-yl)-6-methyl-3-oxo-1,3,9,9a-tetrahydrooxazolo[3,4-a]indol-1-yl)methyl)carbamate;
(1S,9aS)-1-((1H-1,2,3-triazol-1-yl)methyl)-6-fluoro-7-(6-(2-oxooxazolidin-3-yl)pyridin-3-yl)-9,9a-dihydrooxazolo[3,4-a]indol-3 (1H)-one;
N-(((1S,9aS)-6-fluoro-7-(6-(2-methyl-2H-tetrazol-5-yl)pyridin-3-yl)-3-oxo-1,3,9,9a-tetrahydrooxazolo[3,4-a]indol-1-yl)methyl)acetamide;
methyl(((1S,9aS)-6-fluoro-7-(6-(2-methyl-2H-tetrazol-5-yl)pyridin-3-yl)-3-oxo-1,3,9,9a-tetrahydrooxazolo[3,4-a]indol-1-yl)methyl)carbamate;
(1S,9aS)-1-((1H-1,2,3-triazol-1-yl)methyl)-6-fluoro-7-(6-(2-methyl-2H-tetrazol-5-yl)pyridin-3-yl)-9,9a-dihydrooxazolo[3,4-a]indol-3(1H)-one.

Another preferred group of compounds of the present invention is illustrated below:
N-(((1S,9aS)-3-oxo-7-(tetrahydro-2H-thiopyran-4-yl)-1,3,9,9a-tetrahydrooxazolo[3,4-a]indol-1-yl)methyl)acetamide;
N-(((1S,9aS)-3-oxo-7-(1-oxo-tetrahydro-2H-thiopyran-4-yl)-1,3,9,9a-tetrahydrooxazolo[3,4-a]indol-1-yl)methyl)acetamide;
N-(((1S,9aS)-3-oxo-7-(1,1-dioxo-tetrahydro-2H-thiopyran-4-yl)-1,3,9,9a-tetrahydrooxazolo[3,4-a]indol-1-yl)methyl)acetamide;
N-(((1S,9aS)-7-(3,6-dihydro-2H-thiopyran-4-yl)-3-oxo-1,3,9,9a-tetrahydrooxazolo[3,4-a]indol-1-yl)methyl)acetamide;
N-(((1S,9aS)-7-(1-oxo-3,6-dihydro-2H-thiopyran-4-yl)-3-oxo-1,3,9,9a-tetrahydrooxazolo[3,4-a]indol-1-yl)methyl)acetamide;
N-(((1S,9aS)-7-(1,1-dioxo-3,6-dihydro-2H-thiopyran-4-yl)-3-oxo-1,3,9,9a-tetrahydrooxazolo[3,4-a]indol-1-yl)methyl)acetamide;
methyl(((1S,9aS)-3-oxo-7-(tetrahydro-2H-thiopyran-4-yl)-1,3,9,9a-tetrahydrooxazolo[3,4-a]indol-1-yl)methyl)carbamate;
methyl(((1S,9aS)-3-oxo-7-(1-oxo-tetrahydro-2H-thiopyran-4-yl)-1,3,9,9a-tetrahydrooxazolo[3,4-a]indol-1-yl)methyl)carbamate;
methyl(((1S,9aS)-3-oxo-7-(1,1-dioxo-tetrahydro-2H-thiopyran-4-yl)-1,3,9,9a-tetrahydrooxazolo[3,4-a]indol-1-yl)methyl)carbamate;
methyl(((1S,9aS)-7-(3,6-dihydro-2H-thiopyran-4-yl)-3-oxo-1,3,9,9a-tetrahydrooxazolo[3,4-a]indol-1-yl)methyl)carbamate;
methyl(((1S,9aS)-7-(1-oxo-3,6-dihydro-2H-thiopyran-4-yl)-3-oxo-1,3,9,9a-tetrahydrooxazolo[3,4-a]indol-1-yl)methyl)carbamate;
methyl(((1S,9aS)-7-(1,1-dioxo-3,6-dihydro-2H-thiopyran-4-yl)-3-oxo-1,3,9,9a-tetrahydrooxazolo[3,4-a]indol-1-yl)methyl)carbamate;
(1S,9aS)-1-((1H-1,2,3-triazol-1-yl)methyl)-7-(tetrahydro-2H-thiopyran-4-yl)-9,9a-dihydrooxazolo[3,4-a]indol-3(1H)-one;
(1S,9aS)-1-((1H-1,2,3-triazol-1-yl)methyl)-7-(1-oxo-tetrahydro-2H-thiopyran-4-yl)-9,9a-dihydrooxazolo[3,4-a]indol-3(1H)-one;
(1S,9aS)-1-((1H-1,2,3-triazol-1-yl)methyl)-7-(1,1-dioxo-tetrahydro-2H-thiopyran-4-yl)-9,9a-dihydrooxazolo[3,4-a]indol-3(1H)-one;
(1S,9aS)-1-((1H-1,2,3-triazol-1-yl)methyl)-7-(3,6-dihydro-2H-thiopyran-4-yl)-9,9a-dihydrooxazolo[3,4-a]indol-3(1H)-one;
(1S,9aS)-1-((1H-1,2,3-triazol-1-yl)methyl)-7-(1-oxo-3,6-dihydro-2H-thiopyran-4-yl)-9,9a-dihydrooxazolo[3,4-a]indol-3(1H)-one;
(1S,9aS)-1-((1H-1,2,3-triazol-1-yl)methyl)-7-(1,1-dioxo-3,6-dihydro-2H-thiopyran-4-yl)-9,9a-dihydrooxazolo[3,4-a]indol-3(1H)-one.

Additionally, several anti-infective oxazolidinones structures lacking the novel CR₂R₃ group (featured in compounds of the present invention) between the position 4 of the oxazolidinone ring and the ortho (in relation to above ring) position of the N-phenyl substituent have been described in patent publications WO 01/94342, WO 2005/058886, U.S. Pat. No. 6,689,779, WO 97/30995, WO 99/64417, WO 01/40236, WO 01/81350, WO 02/080841, WO 02/081468, WO 02/081469, WO 02/081470, WO 03/072575, WO 03/035648, WO 2004/048350, WO 2004/048370, WO 2004/048392, WO 2004/056816, WO 2004/056817, WO 2004/056818, WO 2004/078753, WO 2004/083205, WO 2004/083206, U.S. Pat. No. 6,734,200, U.S. Pat. No. 6,638,955, U.S. Pat. No. 7,141,583, U.S. Pat. No. 7,157,482, U.S. Pat. No. 7,186,738, U.S. Pat. No. 7,192,974, WO 03/027083, WO 03/048136, WO 2005/005398, 2005/005399, 2005/005420, WO 2005/005422, WO 2005/005398, WO 2004/029066, WO 2004/078770, WO 2005/012270, WO 2005/012271, WO 2005/019211, WO 2005/042554, WO 2005/049632, WO 2005/061468, WO 2005/118610, WO 2006/022794, WO 2006/133397, WO 2007/133803, WO 2004/087697, WO 2004/089943, WO 2004/089944, KR 2001/075920, WO 2004/099199, WO 2004/101552, WO 2004/113329, WO 2005/003087, WO 2005/019213, WO 2005/028473, DE 10342292, WO 2005/042523, WO 2005/054234, WO 2005/070940, WO 2005/082897, WO 2005/082899, WO 2005/082900, WO 2005/113520, WO 2005/116021, WO 2005/116017, WO 2005/116022, WO 2005/116023, WO 2006/010756, US 2006/030609, WO 2006/018682, WO 2006/035283, WO 2006/038100, WO 2006/043121, WO 2006/051408, US 2006105941, WO 2006/056875, WO 2006/056877, KR 2004035207, KR 2004090065, KR 2005020083, KR 2006035035, WO 2006/106427, WO 2006/106426, WO 2006/109156, KR 2006033300, WO 2007/00043, WO 2007/000644, WO 2007/00404, WO 2007/004037, WO 2007/004032, KR 2006092382, IN 2002MU00099, IN 2000MA00369, WO 2007/023507, IN 2004-CH00295, IN 2001DE00827, CN 1948306, IN 2005MU01200, WO 2007/093904, WO 2007/114326, WO 2007/116960, CN 101054374, WO 2007/138381, WO 2008/010070, and WO 2008/014108. It is understood that useful novel derivatives of these structures featuring the new CR₂R₃ group bridge are included within the scope of the present invention. Said novel oxazolidinone derivatives are produced using a combination of techniques described in above publications with general synthetic methods of the present invention (see below, General Synthetic Methods, examples of Schemes 1-13). In addition, the novel compounds of the present invention offer certain advantage(s) over aforementioned prior art analogs (not containing the new group CR₂R₃), such as an improved pharmacological property(ies) in vitro or in vivo, including safety and tolerability. For example, a reduced propensity to undesired monoamine oxidase inhibition (MOA-I) is achieved in compounds of the present invention (see below, MOA-I data for select Examples of the Table 1). For the antibacterial therapeutic agent, lower levels of MAO inhibition are desired, as illustrated by the serotonin syndrome precaution on the prescribing information for the drug Zyvox™, the active ingredient of which is the oxazolidinone linezolid.

In another aspect, the present invention provides methods of synthesis of a compound of formula XXVIII comprising reacting a compound of formula XXVII with a reducing agent to yield a compound of formula XXVIII:

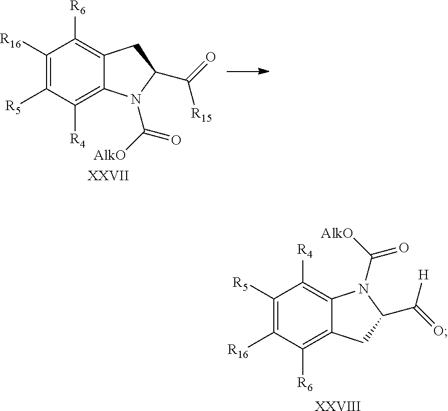

wherein R⁴, R⁵, and R⁶ are independently H, F, Cl, CN, CH₃, or OH; R¹⁵ is OH, N(Me)OMe, OC₁₋₄alkyl, 3 to 6-membered N-heterocycle, or Ar; Alk is C₁₋₄alkyl, C₃₋₆ cycloalkyl, CH₂Ar; and R¹⁶ is H, halo, NH₂, NO₂, R⁷, Het¹, or Het². In certain aspects, the compound of formula XXVII is in enantiomeric excess >85%. In certain aspects, the reducing agent is LiAlH₄, diisobutylaluminum hydride, or a like reducing agent apparent to those of skill in the art. In certain aspects, the yield of the compound of formula XXVIII is at least 50%. In certain aspects, R⁴═R⁵═R⁶═R¹⁶═H; R¹⁵ is N(Me)OMe; and Alk is CH₂Ph. In certain aspects, the compounds of formula XXVII and XXVIII have an absolute configuration and an optical purity of at least 80%.

In another aspect, the present invention provides methods of synthesis of a compound of formula XXIX comprising reacting a compound of formula XXVIII with trialkylsilylcyanide to form the compound of formula XXIX:

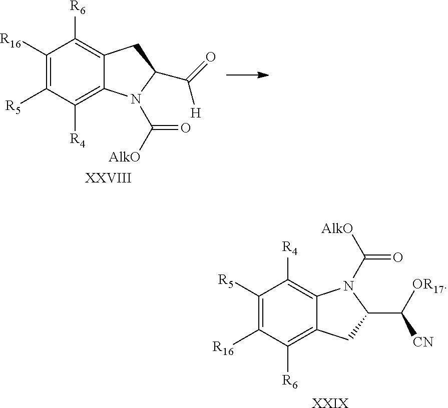

In certain aspects, the reaction of the compound of formula XXVIII in tralkylsilylcyanide is in the presence of LiF, or a like common fluoride source apparent to those of skill in the art. In certain aspects, the reaction of the compound of formula XXVIII in tralkylsilylcyanide is in the presence of HCN, or a like common cyanide source apparent to those of skill in the art. In certain aspects, the reaction of the compound of formula XXVIII in tralkylsilylcyanide is in the presence of a Lewis acid catalyst. In certain aspects, the Lewis acid catalyst is selected from an optically active magnesium (II), aluminum (III), boron (III), lanthanide (III), tin (II), titanium (IV), vanadium (IV), yttrium (IV), or zirconium (IV) complex. In certain aspects, the silicon group on the compound of formula XXIX is optionally removed with an acid or a like reagent apparent to those of skill in the art. In certain aspects, the yield of the compound of formula XXIX is at least 35%. In certain aspects, the compound of formula XXIX has an optical purity of at least 80%. In certain aspects, R⁴═R⁵═R⁶═R¹⁶═H; H; R¹⁵ is N(Me)OMe; and Alk is CH₂Ph. In certain aspects, the compounds of formula XXVIII and XXIX have an absolute configuration and an optical purity of at least 80%.

In another aspect, the present invention provides methods of synthesis of a compound of formula XXX comprising N-deprotecting a compound of formula XXIX, followed by cyclizing the product:

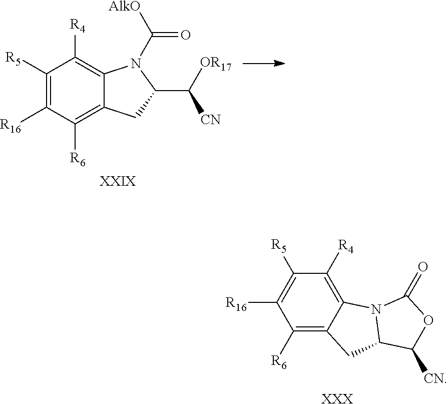

In certain aspects, the compound of formula XXIX is optically active. In certain aspects, the cyclization uses phosgene, or a like reagent apparent to those of skill in the art. In certain aspects, the yield of the compound of formula XXX is at least 50%. In certain aspects, R⁴═R⁵═R⁶═R¹⁶═H; R¹⁵ is N(Me)OMe; and Alk is CH₂Ph. In certain aspects, the compounds of formula XXIX and XXX have an absolute configuration and an optical purity of at least 80%.

### General Synthetic Methods

The compounds of this invention can be prepared in accordance with one or more of the Schemes discussed below. Syntheses of certain specific intermediates are precedented in the prior art. For example, the synthesis of the key intermediate 1 (R₄═R₅═R₆═H) has been described by Gleave and Brickner [J. Org. Chem., 1996, v. 61, pp. 647-6474].

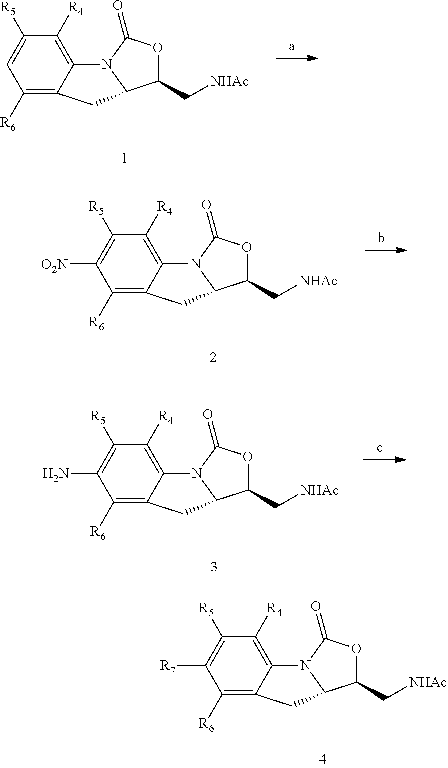

Scheme 1: a) Nitrating agent: HNO₃/H₂SO₄ or HNO₃/trifluoroacetic anhydride, or alike; b) reducing agent: Fe/aq. NH₄Cl or XnCl₂, or alike; c) an appropriate amine group modifying reagent: e.g., O₂S(CH₂CH₂Cl)₂/base (e.g. K₂CO₃) for R₇=thiomorpholine S,S-dioxide; gamma-pyrone for R₇=gamma-pyridone; O(CH₂CH₂Cl)₂/base (e.g. K₂CO₃) for R₇=morpholine; acylating agent C₁₋₄alkyl-COCl/base (e.g., TEA) for R₇═NHCOC₁₋₄alkyl; acylating agent Het¹-COCl/base (e.g., TEA) for R₇═NHCOHet¹.

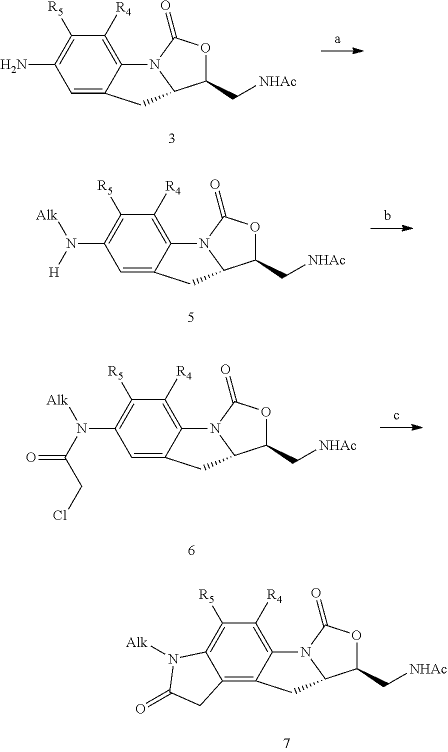

Scheme 2. a) alkylating agent: e.g. AlkCHO/NaBH₃CN/AcOH or alkyl halide/base (e.g., K₂CO₃); b) ClCH₂COCl/base (e.g., K₂CO₃); c) Lewis acid: e.g. TiCl₄, or BF₃ etherate, or AlCl₃, or Yb(OTf)₃; or Pd(II) reagent, such as Pd(OAc)₂ with phosphine or phosphinyl ligand (e.g. 2-[(t-butyl)phosphinyl]biphenyl), base (e.g., TEA).

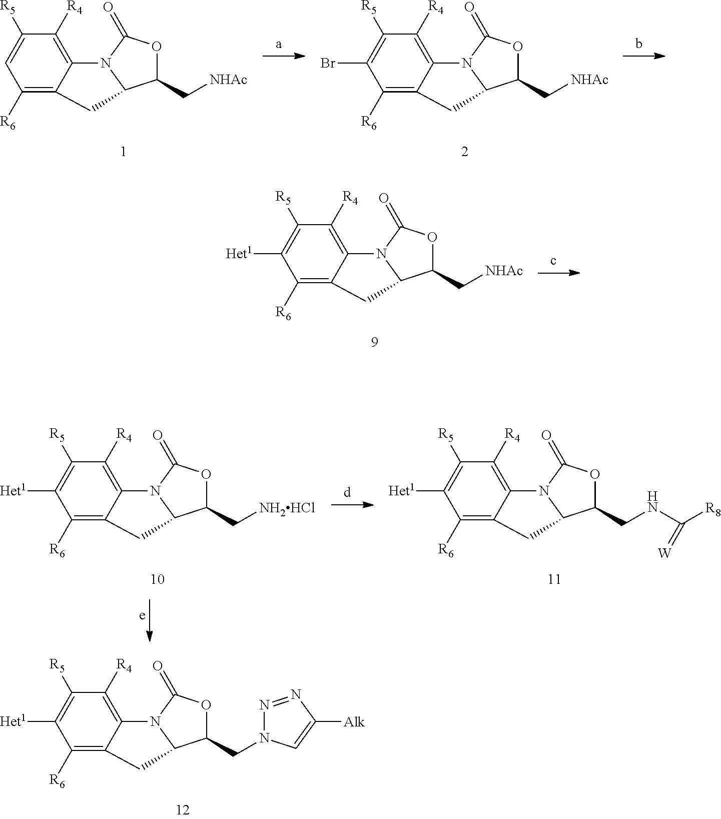

Scheme 3. a) N-Bromosuccinimide (NBS); b) Het¹-B(OH)₂ or Het¹-B(OAlk)₂ e.g. boronic ester picolinate or alike, Pd catalyst (e.g. PdCl₂(dppf)DCM or alike); c) HCl; d) appropriate acylating agent: e.g. R⁸COCl/base (e.g., K₂CO₃)or R⁸COOH/HATU/DIEA for W═O; for R⁸C(═S)SMe/base (e.g., DIEA) for W═S; e) TsNHN═C(CHCl₂)Alk/base (e.g., K₂CO₃).

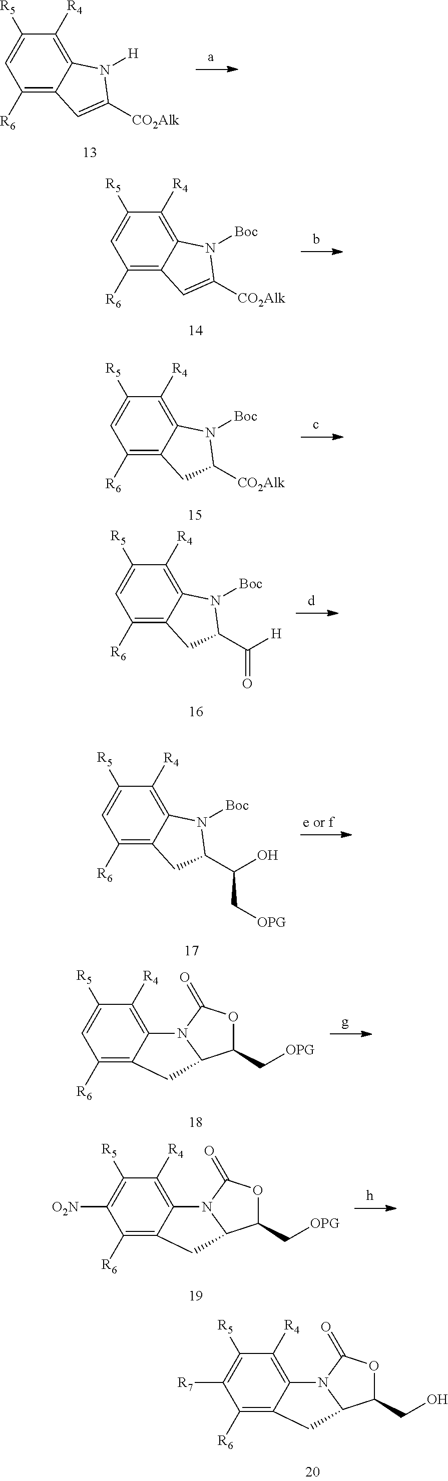

Scheme 5. a) Boc₂O, base (NaH or DMAP); b) [R(nbd)₂]SbF₆-PhTRAP-Cs-2CO₃, H₂, isopropanol; c) DIBAL; d) PGOCH₂MgX (wherein PG is a protective group, such as THP, TBS or alike, and X is Halo; e) base (e.g., DMAP); f) TFA, then N,N′-carbonyldiimidazole, base (e.g., TEA or alike); g) nitrating agent: HNO₃H₂SO₄ or HNO₃/trifluoroacetic anhydride, or alike; h) deprotection: e.g., TFA for PG=THP, or TBAF for PG=TBS.

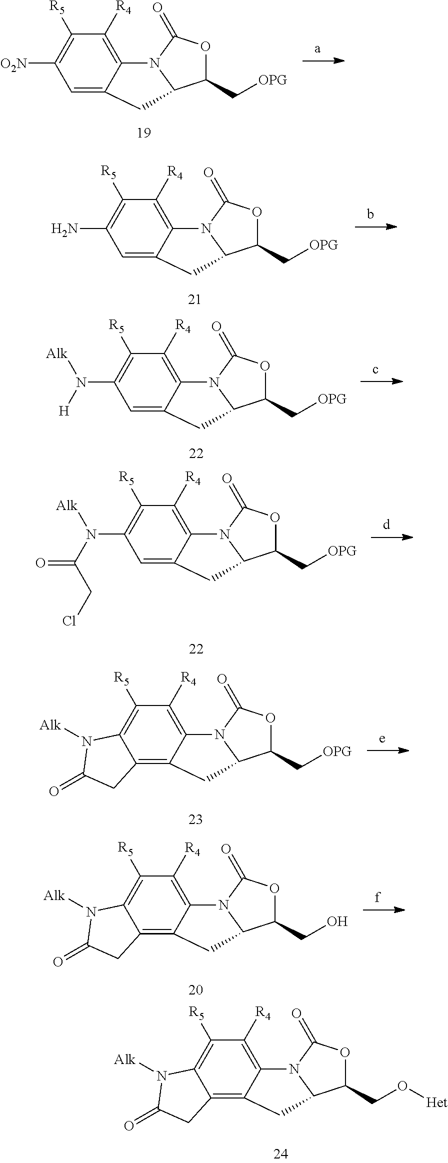

Scheme 6. a) Reducing agent (e.g. Fe/NH₄Cl); b) alkylating agent: e.g. AlkCHO/NaBH₃CN/AcOH or alkyl halide/base (e.g., K₂CO₃); c) ClCH₂COCl/base (e.g., K₂CO₃); d) Pd(II) reagent, such as Pd(OAc)₂ with phosphine or phosphinyl ligand (e.g. 2-[(t-butyl)phosphinyl]biphenyl), base (e.g., TEA); e) deprotection: e.g., TFA for PG=THP, or TBAF for PG=TBS; f) Het¹OH or Het²OH, Mitsunobu reagents (e.g., triphenylphosphine, DIAD, base).

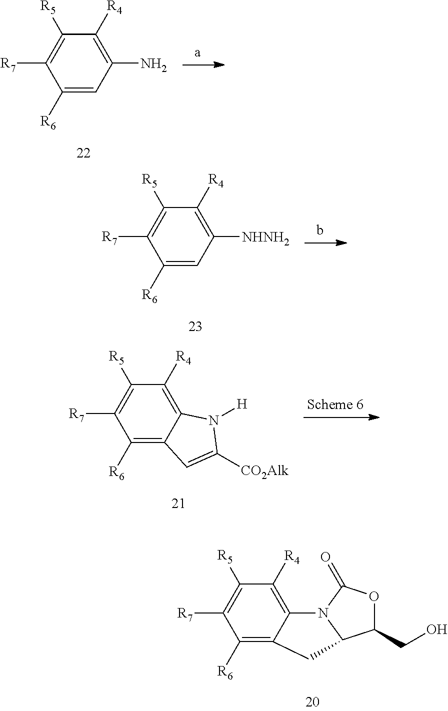

Scheme 7. a) NaNO₂, HCl, then SnCl₂; or ArSO₂ONH₂; b) MeC(═O)CO₂Alk, heating (Fisher indole synthesis).

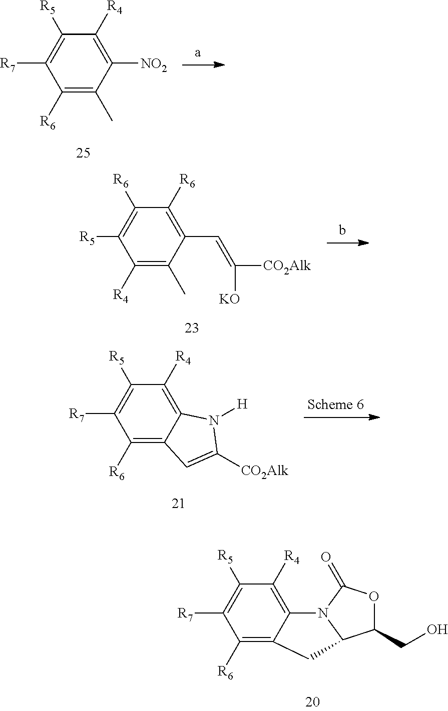

Scheme 8. a) CH₂(CO₂Alk)₂, KOH; b) H₂, PtO₂.

In one preferred embodiment, the synthesis of the compounds of formula I is performed using optically active (S)-indoline-2-carboxylic acid derivatives as shown in Scheme 9 below. Specific N-Cbz group at the indoline nitrogen is provided herein as an example only, and can be substituted for a multitude of common alkyl carbamate groups (e.g., i-Pr, Me, or Et carbamate). The Scheme is generally applicable to a multitude of variously substituted aromatic derivatives 26 to arrive at respective compounds of formula I exemplified herein by structures 37, 39, and 42.

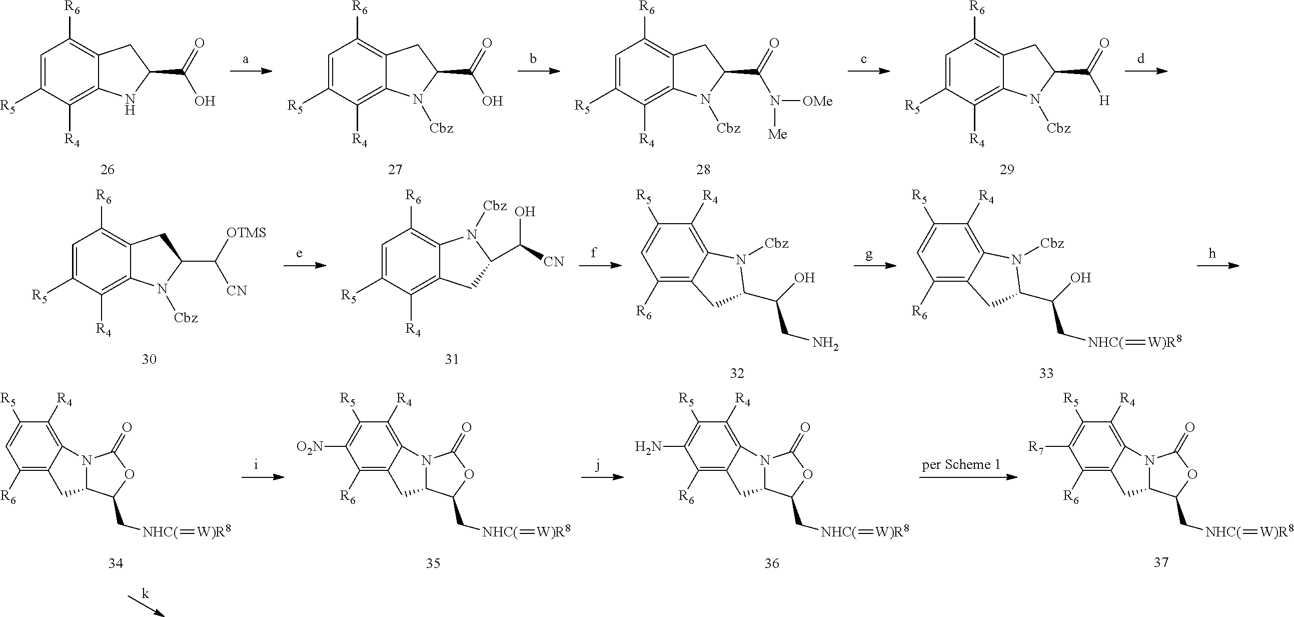

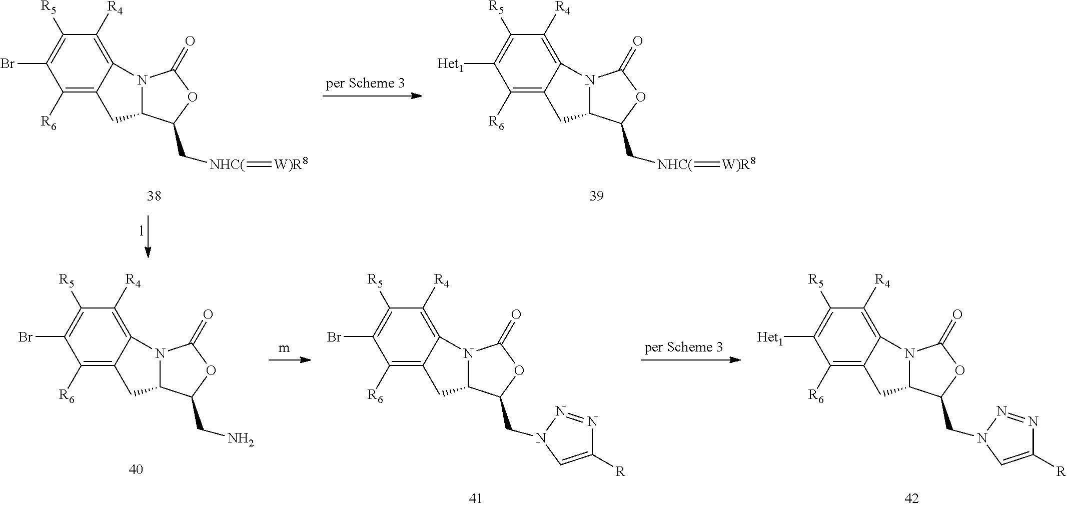

Scheme 9. a) Cbz-Cl, DIEA; b) carbonyldiimidazole (CDI), MeNHOMe, DIEA; c) reduction: e.g., LAH or DIBAL-H; d) TMSCN, LiF; e) deprotection: e.g. TFA or TBAF; Reduction: e.g. BH₃Me₂S or H₂, Pt/C or Pd/C; g) acylating agent: e.g. R⁸COCl/base (e.g., K₂CO₃) or R⁸COOH/HATU/DIEA for W═O; for R⁸C(═S)SMe/base (e.g., DIEA) for W═S; h) base, e.g. K₂CO₃; i) HNO₃; j) reduction: H₂, Pt/C or Pd/C; or Fe/NH₄Cl; k) NBS; 1) for R⁸C(═W)=Boc: TFA; for R⁸C(═W)=Ac: aq. HCl or aq. —alcohol HCl; then base (e.g., K₂CO₃); m) TsNHN═C(CHCl₂)R/base (e.g., K₂CO₃).

In another preferred embodiment, the synthesis of the compounds of formula I is performed as illustrated in Scheme 10. This synthesis is based on the chiral cyanohydrine derivatives 31 of the preceding Scheme 9. Alternatively, racemic (at indoline CH) cyanohydrine analogs of 31 can be cyclized into respective racemic oxazolidinone analogs of 37 and then separated by conventional means (such as column chromatography or chrystallization) to afford the desired compounds 32. The latter intermediates are then transformed into the desired compounds of formula I as per Scheme 10 below.

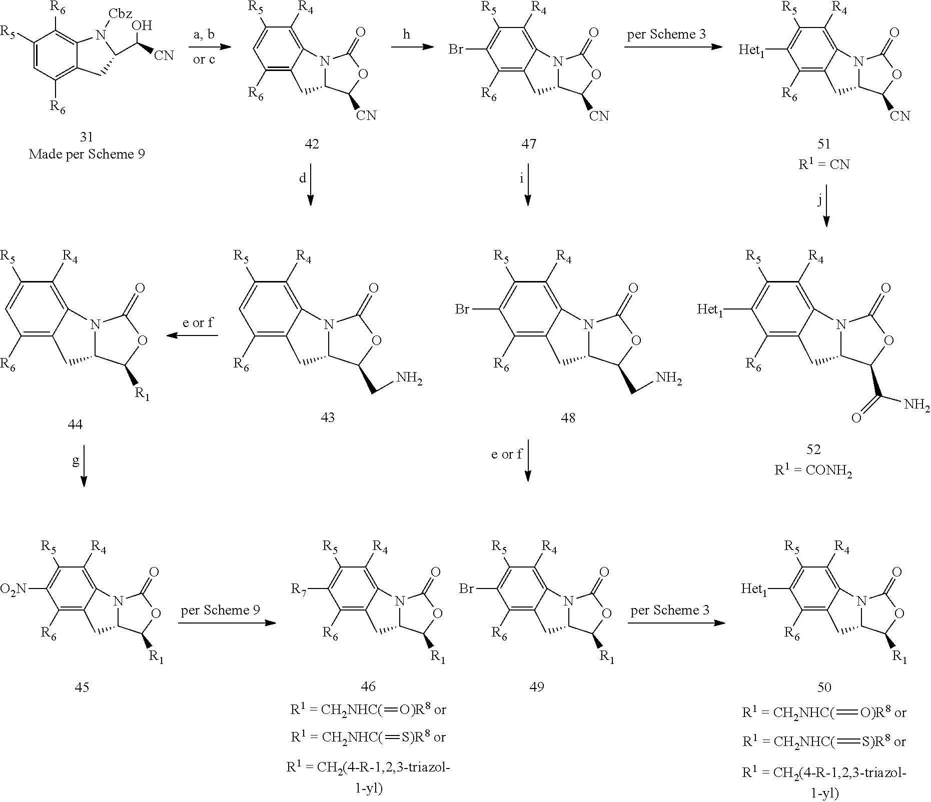

Scheme 10. a) H₂, Pd/C; b) COCl₂, carbonyldiimidazole, or triphosgene, base (e.g., TEA); c) base (e.g. K₂CO₃, LiOBu-t, DMAP); d) reducing agent: e.g. H₂ with Pd/C or Pt/C; or BH₃.Me₂S; e) for R¹═CH₂NHC(═W)R⁸: acylating agent: e.g. R⁸COCl/base (e.g., K₂CO₃) or R⁸COOH/2-(1H-7-Azabenzotriazol-1-yl)—1 μl, 3,3-tetramethyl uronium hexafluorophosphate(HATU)/N,N-diisopropylethylamine (DIEA) for W═O; for R⁸C(═S)SMe/base (e.g., DIEA) for W═S; f) for R¹=4-R-1,2,3-triazol-1-yl: TsNHN═C(CHCl₂)R/base (e.g., K₂CO₃); g) nitrating agent: HNO₃/H₂SO₄ or HNO₃/trifluoroacetic anhydride, or alike; h) N-bromosuccinimide; i) reducing agent: e.g. BH₃.Me₂S or alike; j) nitrile-hydrolyzing agent: e.g. aq. H₂SO₄ or aq. HCl, or TMSCl, aq. alcohol; or H₂O₂, LiOH or KOH.

Importantly, invented herein synthetic methods of above Schemes 9 and 10 employ new chemistry and are economically advantageous vs. the prior art synthesis involving asymmetric starting materials. Specifically, the method of present invention is superior to that of the U.S. Pat. No. 5,231,188 in terms of the number of synthetic steps, the process duration, and separation techniques required. In particular, the prior art necessitated an indole 2-carboxylate reduction into an achiral mixture of indoline 2-carboxylate derivative, not required herein. Furthermore, at the stage of achiral indoline analog of the amine 32 (of Scheme 9), a laborious enantiomer separation via crystallization with an asymmetric auxiliary reagent was required in the method by Brickner et al. as described in U.S. Pat. No. 5,231,188 (no yields provided therein). In contrast, only simple chromatographic separation is needed in the method of this invention to isolate the key optically pure intermediates 31.

Moreover, the method of Scheme 9 utilizes unprecedented and highly efficient synthesis of cyanohydrines 31 via a facile formation of a silicon ether precursor 30 using trimethylsilyl cyanide (TMSCN) and LiF. In contrast to the acetone cyanohydrine method of the U.S. Pat. No. 5,231,188 featuring a 3-day process, the new method of the present invention employs a commercially available reagent TMSCN (or any other (trisubstituted)silyl cyanide) to afford the desired intermediate in quantitative yields after only 1-12 h reaction under ambient conditions. The method also permits for a use of chiral catalysts (e.g. taddol—Ti(IV), tartrate—Ti(IV), or triol—Ti(IV) complexes, asymmetric boron catalysts, and alike) employed at the cyanohydrine formation stage to afford the desired stereoisomer in a stereoselective manner (as reviewed for asymmetric cyanohydrine syntheses with TMSCN by Brunel and Holmes in Angew. Chemie, 2004, 2753). Other than TMSCN cyanide sources may be used likewise for the asymmetric formation of cyanohydrins 31. The new methods of Schemes 9 and 10 also compares favorably to a different (Sharpless oxidation) multi-step asymmetric route to related intermediates described by Gleave and Brickner in J. Org. Chem., 1996, v. 61, pp. 647-6474.

Certain preferred fluorinated compounds of the formula I can be made either using respective fluorine-substituted starting materials per Schemes 1-10 (if one, two, or all of R₄, R₅, or R₆ are F), or produced via a direct fluorination of aniline derivatives (such as compounds 3 and 36 of Schemes 1 and 9), or amide (e.g. trifluoroacetamide) derivatives thereof (e.g., using CF₃OF as described by Fifolt in U.S. Pat. No. 4,766,243). Alternatively, these can be produced per Scheme 11, via a 2-step process involving an ortho-lithiation of a respective alkyl carbamate derivatives (as described by Pinto et al. in Organic Letters, 2006, p. 4929), followed by a fluorination using one of several commercially available electrophilic fluorine sources, such as Selectfluor^{R} (Chung et al. in J. Fluorine Chem., 2004, p. 543), N-fluorobenzenesulfonamide (Aldrichimica Acta, 1995, vol. 28, p. 36), CF₃OF (Middleton et al. in J. Am. Chem. Soc., 1980, p. 4845), N-fluoropyridinium salts (Umemoto et al. in J. Am. Chem. Soc., 1990, p. 8563), or alike reagents capable of a carbanion fluorination (see e.g. Umemoto et al. in J. Am. Chem. Soc., 1990, p. 8563, and references cited therein).

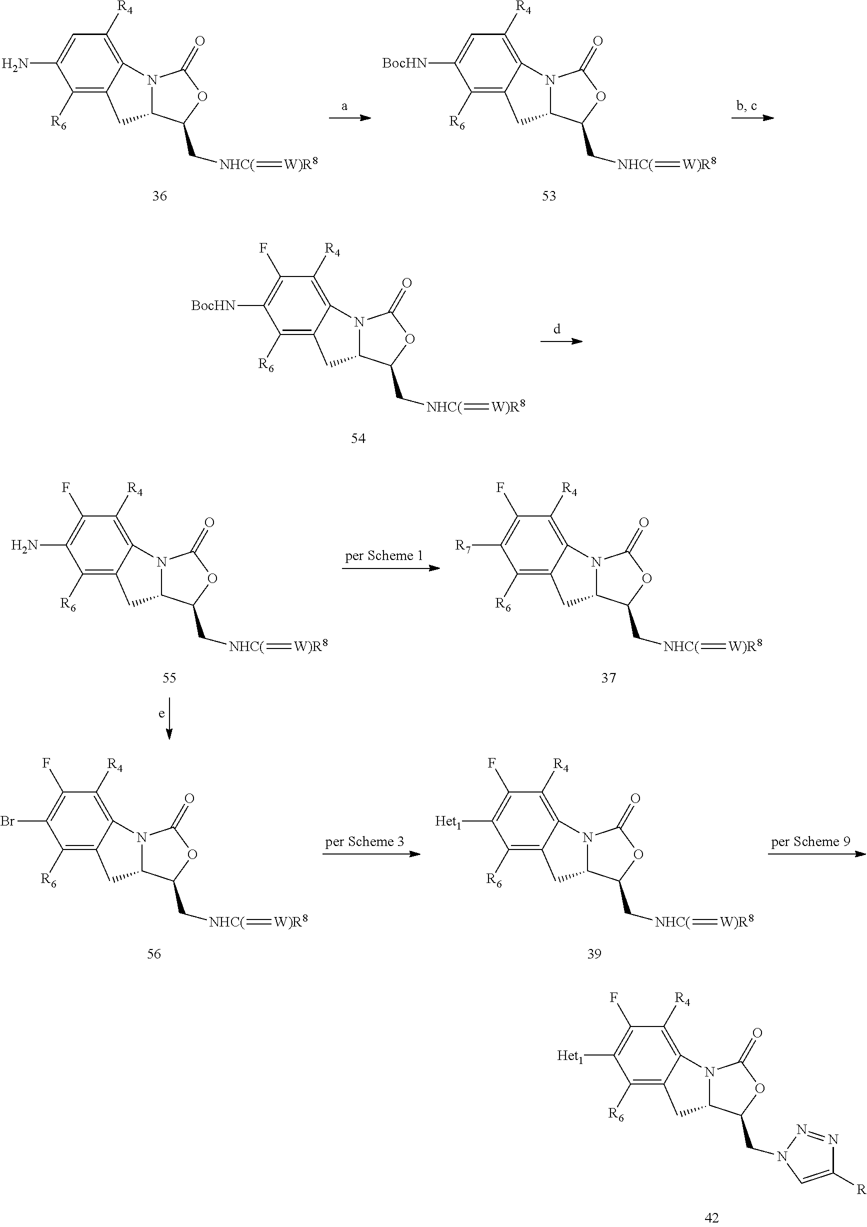

Scheme 11. a) Boc₂O, DIEA; b) base: e.g. sec-BuLi, sec-BuLi/N,N,N′,N′-tetrameylenediamine, or tert-BuLi; c) fluorinating reagent: e.g. Selectfluor^{R}, N-fluorobenzenesulfonamide, N-fluoropyridinium triflate or alike; d) TFA; e) NaNO₂, HBr or tert-BuONO, CuBr₂.

It is to be noted that intermediates of the type 22 (aniline) and 24 (nitroarene) of Schemes 7 and 8 are ubiquitous in oxazolidinone literature, and variety of these bearing a multitude of suitable R⁴-R⁷ substituents can be prepared as described, e.g., in the following publications: WO 9323384, WO 20028084, WO 2003072553, WO 2003072576, WO 2003072575, WO 200142229, WO 200264575, WO 9615130, WO 200216960, WO 200027830, WO 200146185, WO 200281469, WO 200281470, WO 2001080841, WO 2003084534, WO 2003093247, WO 200202095, WO 200230395, WO 200272066, WO 2003063862, WO 2003072141, WO 2003072081, WO 2003119817, WO 2003008389, WO 2003007870, WO 200206278, WO 200032599, WO 9924428, WO 2004014392, WO 2004002967, WO 2004009587, WO 2004018439, WO 2004074282, US Patent Application Publication No. US 2004/0044052, U.S. Pat. No. 5,547,950, U.S. Pat. No. 5,700,799, DE 10034627. Furthermore, the hydroxymethyl group (R¹═CH₂OH) of compounds 20 can be transformed into a variety of desired R¹ substituents just as described, for example, in aforementioned publications.

Select compounds I of the present invention can be produced and utilized in a prodrug form to maximize certain useful pharmarcological properties such as aqueous solubility for injections, or oral bioavailability for administration in a tablet, powder, or gel forms. Various prodrug forms can be made and employed, such as carboxylic acid and amino acid esters, carbamates, or phosphate ester derivatives known in the art (for a review, see e.g. Ettmayer et al. J. Med. Chem., 2004, p. 2393). An example for general synthesis of such compounds and phosphate prodrugs thereof is illustrated by Scheme 12. Specific preferred boronic ester of the intermediate 57 shown herein as an example only, and can be substituted by a variety of similar derivatives known in the carbon-carbon bond forming chemistry, such as boronic acid, tin derivatives, etc. (for a review, see e.g. Rossi et al. Synthesis, 2004, p. 2419).

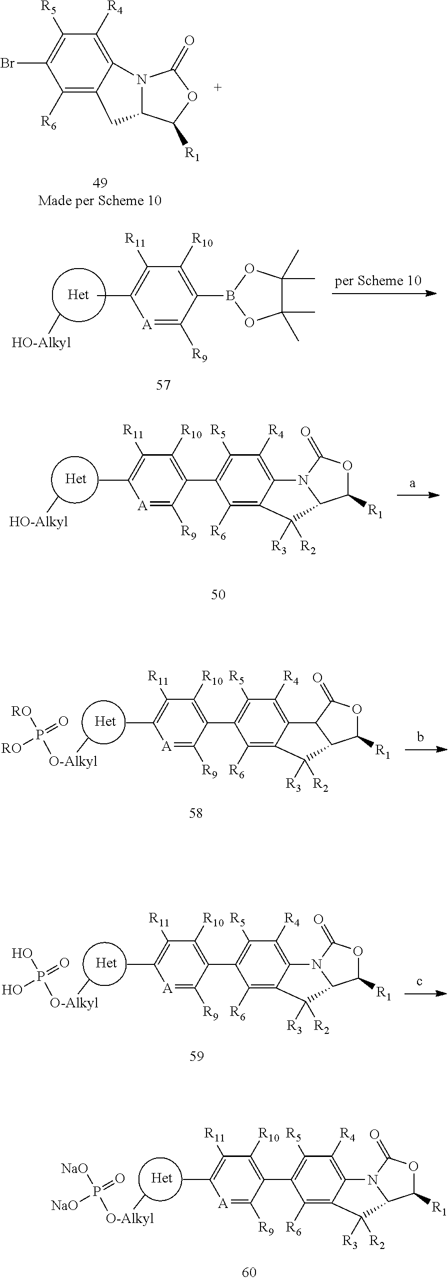

Scheme 12. a) Phosphorylating reagents: e.g., pyrophosphate [(RO)₂P(═O)]₂O, base (e.g., NaH, LDA, or 1,8-diazabicyclo[5.4.0]undec-7-ene (DBU); b) deprotection: for R═PhCH₂: H₂, Pd/C; for R=t-Bu: trifluoroacetic acid (TFA); c) basic sodium source: e.g. NaOH, NaOAc, NaHCO₃, or Na₂CO₃.

Additional prodrug derivatives have been reported in WO 05/028473. Likewise, certain prodrugs of compounds of the present invention can be prepared herein as shown in Scheme 13, wherein Alk₁ and Alk₂ are alkyl groups, most commonly being C₁₋₄ alkyl and alike.

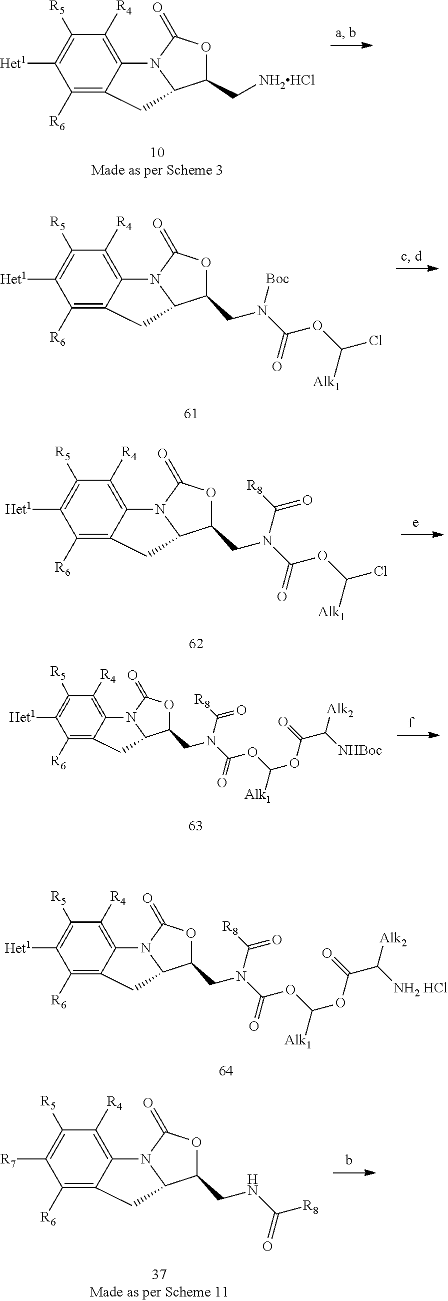

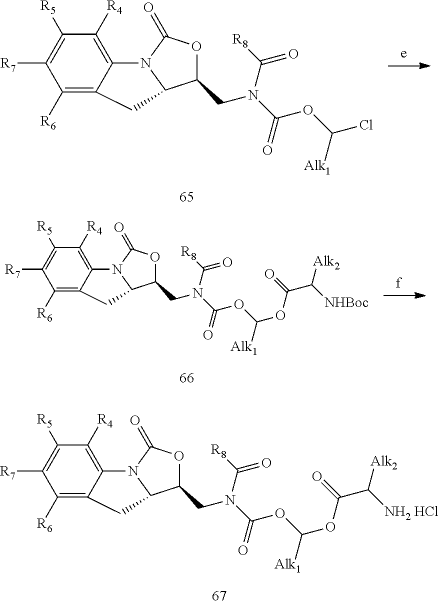

Scheme 13. a) (t-BuOC(═O))₂O (Boc₂O), Boc-ON, or alike; base (e.g., NaHCO₃, DIEA, or alike); b) Cl(C═O)OCH(Alk₁)Cl, base (e.g., NaHCO₃, LiOBu-t, DIEA, or 1,8-diazabicyclo[5.4.0]undec-7-ene (DBU); c) trifluoroacetic acid (TFA); d) acylating agent: e.g. R⁸C(═O)Cl, R⁸OC(═O)C1, or R⁸C(═O)OH, HATU or HBTU, DIEA; e) BocNHCH(Alk₂)COOH metal salt (e.g. Na, K, Cs, or Ag salt), optional NaI, KI, or CsI; f) HCl in dioxane, ether, or alike solvent.

### EXAMPLES

Embodiments of the present invention are described in the following examples, which are meant to illustrate and not limit the scope of this invention.

### Example 1

### Preparation of N-(((1S,9aS)-7-(6-(2-methyl-2H-tetrazol-5-yl)pyridin-3-yl)-3-oxo-1,3,9,9a-tetrahydrooxazolo[3,4-a]indol-1-yl)methyl)acetamide

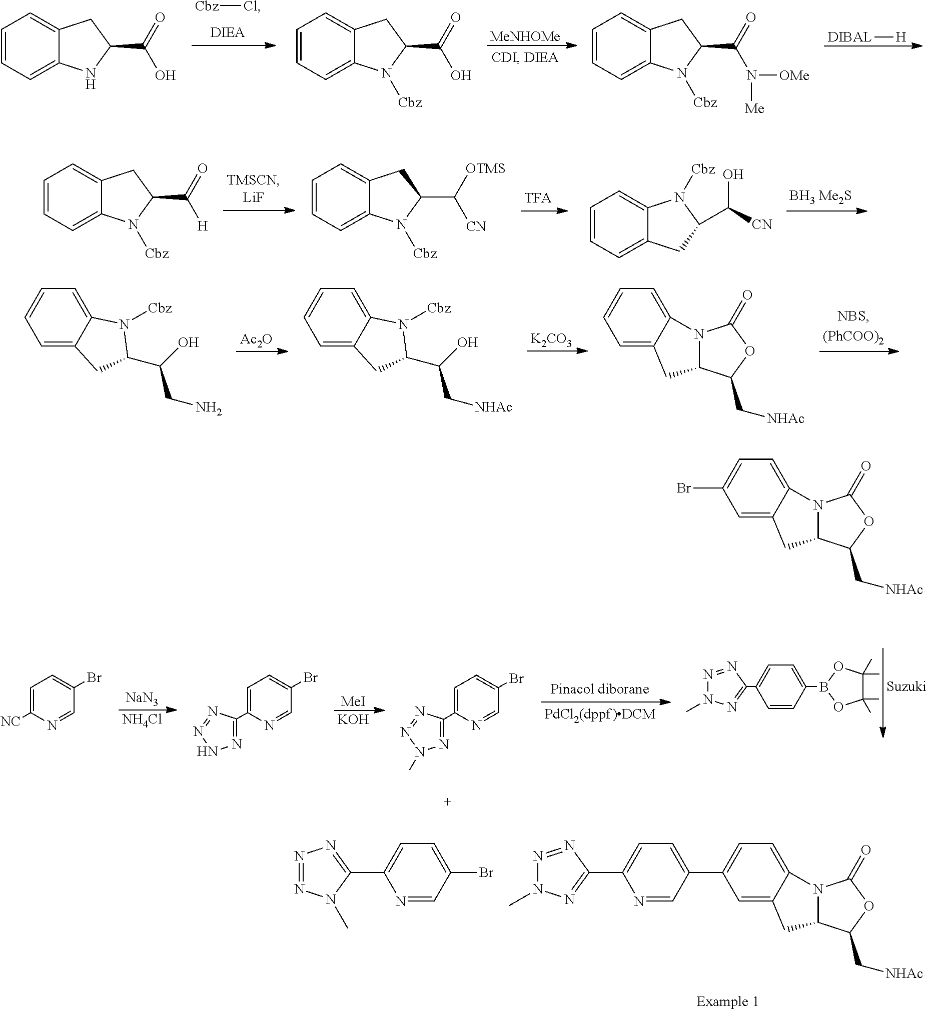

### Step 1. Preparation of (S)-1-(benzyloxycarbonyl)indoline-2-carboxylic acid

Cbz-Cl (20 mL, 0.13 mol) in MeCN (50 mL) was added dropwise to (S)-indoline-2-carboxylic acid (20 g, 0.12 mol) and DIEA (43 mL, 0.25 mol) in MeCN (350 mL) at 5-10° C. over 20 min. The mixture was allowed to warm up r.t. After 3 h, volatiles were removed under vacuum. The oily residue was dissolved in EtOAc and washed with 1% aq. HCl, water, brine, and dried (MgSO₄). Solvent was removed under vacuum to afford the product as a thick oil that crystallized in refrigerator into a brown solid. Yield 35 g (100%).

### Step 2. Preparation of (S)-benzyl 2-(methoxy(methyl)carbamoyl)indoline-1-carboxylate

CDI (14.2 g, 0.087 mol) was added to (S)-1-(benzyloxycarbonyl)indoline-2-carboxylic acid (20 g, 0.067 mol) in DCM (150 mL) at −5° C., and the solution was kept at −5° C. for 1 h. DIEA (15.3 mL, 0.087 mol) was added, followed by N,O-dimethylhydroxylamine hydrochloride (8.5 g, 0.087 mol). The mixture was allowed to warmed up to r.t. and stirred for 30 min, then filtered and washed with EtOAc to obtain (S)-benzyl 2-(methoxy(methyl)carbamoyl)indoline-1-carboxylate as a white solid (20 g, yield: 88%). [α]₂₁D−73.3° (C=0.5, DCM).

### Step 3. Preparation of (S)-benzyl 2-formylindoline-1-carboxylate

DIBAL-H in toluene (353 mL, 0.35 mol) was added dropwise with stirring under Ar to (S)-benzyl 2-(methoxy(methyl)carbamoyl)indoline-1-carboxylate (40 g, 0.12 mol) in dry THF (800 mL) at −78° C. over 30 min. The reaction mixture was stirred at −78° C. for 1 h, then allowed to warm up to −30° C. over 1 h and stirred for an additional 30 min. Cold MeOH (20 mL) was added, then 2M HCl (200 mL) was added. The product was extracted with EtOAc (1000 mL). The organic layer was washed with 2M HCl (2×200 mL) and brine (200 mL), and dried (MgSO₄). Solvent was evaporated under vacuum to afford an oil. Purification by chromatography (silica gel, petroleum ether:EtOAc=8:1) afforded the title compound as a yellow solid (27.2 g, yield: 82%).

### Step 4. Preparation of (2S)-benzyl 2-(cyano(trimethylsilyloxy)methyl)indoline-1-carboxylate

(S)-Benzyl 2-formylindoline-1-carboxylate (25.9 g, 0.092 mol) and LiF (3.5 g, 0.183 mol) were dissolved in THF (200 mL) under Ar, then TMSCN (17 mL, 0.183 mol) was added with stirring. The resulting was stirred at r.t. for 5 h under Ar. Volatiles were removed under vacuum to afford the product as an oil, which was directly used in the next step without further purification.

### Step 5. Preparation of (S)-benzyl 2-((R)-cyano(hydroxy)methyl)indoline-1-carboxylate

TFA (38 mL) was added dropwise with stirring to a solution of (2S)-benzyl 2-(cyano(trimethylsilyloxy)methyl)indoline-1-carboxylate in THF, and the mixture was stirred at r.t. for 12 h. Excess of EtOH was added, and the volatiles removed to afford an oil. Purification by chromatography (silica gel, petroleum ether:EtOAc 8:1) afforded a solid, which was crystallized with petroleum ether/EtOAc: 12/1 to afford desired (S)-benzyl 2-((R)-cyano(hydroxy)methyl)indoline-1-carboxylate [10 g, yield: 35% (for two steps)]. [α]₂₁D−46.9° (C=0.5, EtOH). MS (m/z): 309 [M+H]⁺.

### Step 6. Preparation of (S)-benzyl 2-((S)-2-amino-1-hydroxyethyl)indoline-1-carboxylate

2 M Borane-dimethylsulfide in THF (24 mL, 48 mmol) was added to a solution of (S)-benzyl 2-((R)-cyano(hydroxy)methyl)indoline-1-carboxylate in dry THF (50 mL) at r.t. The reaction mixture was heated to reflux and stirred for an additional 30 min, then which was cooled to r.t. and directly used in the next step without further purification.

### Step 7. Preparation of (S)-benzyl 2-((S)-2-acetamido-1-hydroxyethyl)indoline-1-carboxylate

Ac₂O (40 mL) was added to the solution of the (S)-benzyl 2-((S)-2-amino-1-hydroxyethyl)indoline-1-carboxylate with stirring. Agitation was continued for 2 h. The solvent was removed under vacuum, and the product purified chromatography (silica gel, EtOAc) to afford the product as a white solid. Yield 8.8 g, 77% (for two steps)]. MS (m/z): 355 [M+H]⁺.

### Step 8. Preparation of N-(((1S,9aS)-3-oxo-1,3,9,9a-tetrahydrooxazolo[3,4-a]indol-1-yl)methyl)acetamide

To a solution of (S)-benzyl 2-((S)-2-acetamido-1-hydroxyethyl)indoline-1-carboxylate (10 g, 28.2 mmol) in MeCN (100 mL) was added K₂CO₃ (3.9 g, 28.2 mmol), and the reaction mixture was stirred at 45° C. overnight. Filtration, concentration and purification by chromatography (silica gel, EtOAc) afforded the title compound as a white solid. Yield 6.6 g, 96%). [α]₂₁D−62.4° (C=0.44, DCM). MS (m/z): 247 [M+H]⁺.

### Step 9. Preparation of N-(((1S,9aS)-7-bromo-3-oxo-1,3,9,9a-tetrahydrooxazolo[3,4-a]indol-1-yl)methyl)acetamide

To a solution of N-(((1S,9aS)-3-oxo-1,3,9,9a-tetrahydrooxazolo[3,4-a]indol-1-yl)methyl)acetamide (9.5 g, 0.039 mol) in MeCN (100 mL)was added N-bromosuccinimide (8.9 g, 0.05 mol) and (PhCOO)₂ (0.9 g, 3.9 mmol). After having been stirred at r.t. overnight, the solution was evaporated under vacuum. Purification by chromatography (silica gel, EtOAc) afforded the title compound (9.5 g, yield: 76%). [α]₂₂D−22.9° (C=0.5, DCM).

### Step 10. Preparation of 5-bromo-2-(2H-tetrazol-5-yl)pyridine

To the solution of 5-bromopicolinonitrile (2.33 g, 12.7 mmol) in DMF (26 mL) was added NH₄Cl (2.18 g, 40.7 mmol) and NaN₃ (1.24 g, 19.1 mmol) at r.t. and the mixture was heated to 120° C. for 4 h. The reaction mixture was poured into ice water (100 mL) and acidified to pH=2 with 6N HCl, stirred for 1 h, and the mixture was extracted with EtOAc. The organic phases were combined, dried (Na₂SO₄) and evaporated under vacuum to give crude 5-bromo-2-(2H-tetrazol-5-yl)pyridine in 3.15 g, which was used for next step without further purification.

### Step 11. Preparation of 5-bromo-2-(2-methyl-2H-tetrazol-5-yl)pyridine

To the solution of crude 5-bromo-2-(2H-tetrazol-5-yl)pyridine (3.15 g, 13.9 mmol) in DMF (32 mL) was added MeI (7.92 g, 55.8 mmol) and KOH (1.95 g, 34.8 mmol) at room temperature. The mixture was stirred for 23 h at r.t. The reaction mixture was poured into ice water (100 mL) and extracted with EtOAc. The organic layer was washed with brine, dried (Na₂SO₄), and evaporated under vacuum to give a residue, further purified by flash column chromatography (hexanes—EtOAc from 50:1 to 10:1) to give 5-bromo-2-(2-methyl-2H-tetrazol-5-yl)pyridine as yellow solid (1.32 g, 43% yield over 2 steps) and 5-bromo-2-(1-methyl-1H-tetrazol-5-yl)pyridine as a white solid (0.97 g, 32% yield, 2 steps).

### Step 12. Preparation of 2-(2-methyl-2H-tetrazol-5-yl)-5-(4,4,5,5-tetramethyl-1,3,2-dioxaborolan-2-yl)pyridine

To the solution of 5-bromo-2-(2-methyl-2H-tetrazol-5-yl)pyridine (480 mg, 2 mmol) in DMSO (5 mL) was added pinacol diborane (1.02 g, 4 mmol), KOAc (588 mg, 6 mmol) and PdCl₂(dppf)DCM (160 mg, 0.2 mmol), degassed with N₂. The mixture was stirred at 80° C. for 2 h. The reaction mixture was diluted with DCM (100 mL) and washed with brine (2×100 mL), dried (Na₂SO₄) and evaporated under vacuum, then purified by prep-TLC (hexanes—EtOAc) to give 2-(2-methyl-2H-tetrazol-5-yl)-5-(4,4,5,5-tetramethyl-1,3,2-dioxaborolan-2-yl)pyridine as white solid (380 mg, 66% yield).

### Step 13. Preparation of N-(((1S,9aS)-7-(6-(2-methyl-2H-tetrazol-5-yl)pyridin-3-yl)-3-oxo-1,3,9,9a-tetrahydrooxazolo[3,4-a]indol-1-yl)methyl)acetamide

To the solution of 2-(2-methyl-2H-tetrazol-5-yl)-5-(4,4,5,5-tetramethyl-1,3,2-dioxaborolan-2-yl)pyridine (128 mg, 0.45 mmol) in dioxane/H₂O (5:1, 5 mL) was added N-(((1S,9aS)-7-bromo-3-oxo-1,3,9,9a-tetrahydrooxazolo[3,4-a]indol-1-yl)methyl)acetamide (100 mg, 0.31 mmol), K₂CO₃ (128 mg, 0.93 mmol) and PdCl₂(dppf)DCM (25 mg, 0.03 mmol), degassed and protected with N₂. The mixture was stirred at 80° C. for 3 h. The reaction mixture was diluted with DCM/MeOH (2:1, 20 mL), filtered and evaporated under vacuum, then purified by preparative TLC (2-10% MeOHin DCM) to give N-(((1S,9aS)-7-(6-(2-methyl-2H-tetrazol-5-yl)pyridin-3-yl)-3-oxo-1,3,9,9a-tetrahydrooxazolo[3,4-a]indol-1-yl)methyl)acetamide as white solid (110 mg, 87% yield). ¹H NMR (DMSO-d₆, 300 MHz): 9.12-9.11 (m, 1H), 8.36-8.27 (m, 3H), 7.80-7.74 (m, 2H), 7.40-7.37 (m, 1H), 4.77-4.74 (m, 1H), 4.57-4.55 (m, 1H), 4.45 (s, 3H), 3.56-3.52 (m, 2H), 3.27-3.17 (m, 2H), 1.88 (s, 3H); MS (m/z): 406.1 [M+H]⁺.

### Example 2

### Preparation of N-(((1S,9aS)-7-(6-(1-methyl-1H-tetrazol-5-yl)pyridin-3-yl)-3-oxo-1,3,9,9a-tetrahydrooxazolo[3,4-a]indol-1-yl)methyl)acetamide

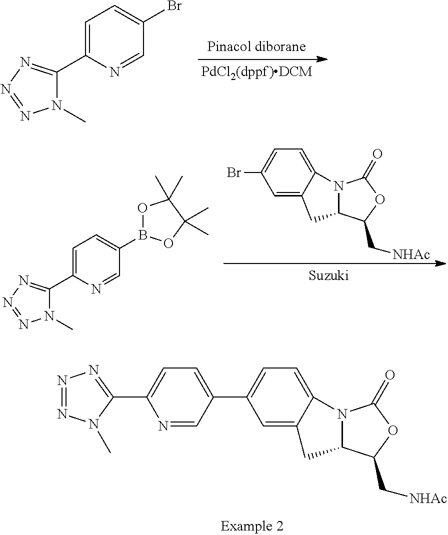

### Step 1. Preparation of 2-(1-methyl-1H-tetrazol-5-yl)-5-(4,4,5,5-tetramethyl-1,3,2-dioxaborolan-2-yl)pyridine

To the solution of 5-bromo-2-(1-methyl-1H-tetrazol-5-yl)pyridine (200 mg, 0.83 mmol, prepared as described in Example 1, Step 11) in dioxane (2 mL) was added pinacol diborane (270 mg, 1.06 mmol), KOAc (270 mg, 2.75 mmol) and PdCl₂(dppf)DCM (60 mg, 0.07 mmol), degassed and protected with N₂. The mixture was stirred at 80° C. for 2 h. The reaction mixture was diluted with DCM (100 mL) and washed with brine (2×100 mL), dried (Na₂SO₄) and evaporated under vacuum, then purified by preparation TLC to give 2-(1-methyl-1H-tetrazol-5-yl)-5-(4,4,5,5-tetramethyl-1,3,2-dioxaborolan-2-yl)pyridine as white solid (170 mg, 71% yield).

### Step 2 N-(((1S,9aS)-7-(6-(1-methyl-1H-tetrazol-5-yl)pyridin-3-yl)-3-oxo-1,3,9,9a-tetrahydrooxazolo[3,4-a]indol-1-yl)methyl)acetamide

To the solution of 2-(1-methyl-1H-tetrazol-5-yl)-5-(4,4,5,5-tetramethyl-1,3,2-dioxaborolan-2-yl)pyridine (99 mg, 0.34 mmol) in dioxane/H₂O (5:1, 5 mL) was added N-(((1S,9aS)-7-bromo-3-oxo-1,3,9,9a-tetrahydrooxazolo[3,4-a]indol-1-yl)methyl)acetamide (86 mg, 0.26 mmol), K₂CO₃ (110 mg, 0.79 mmol) and PdCl₂(dppf)DCM (22 mg, 0.03 mmol), degassed and protected with N₂. The mixture was stirred at 80° C. for 3 h. The reaction mixture was diluted with DCM—MeOH 2:1 (20 mL), filtered and evaporated under vacuum, then the residue was purified by preparative TLC (hexanes—EtOAc) to give N-(((1S,9aS)-7-(6-(1-methyl-1H-tetrazol-5-yl)pyridin-3-yl)-3-oxo-1,3,9,9a-tetrahydrooxazolo[3,4-a]indol-1-yl)methyl)acetamide as white solid (83 mg, 78% yield). ¹H NMR (CDCl₃, 300 MHz): 8.94-8.93 (m, 1H), 8.30-8.27 (m, 1H), 8.01-7.98 (m, 1H), 7.54-7.49 (m, 3H), 6.11-6.07 (m, 1H), 4.66-4.56 (m, 2H), 4.46 (s, 3H), 3.82-3.79 (m, 1H), 3.74-3.67 (m, 1H), 3.44-3.36 (m, 1H), 3.24-3.15 (m, 1H), 2.07 (s, 3H); MS (m/z): 406.1 [M+H]⁺.

### Example 3

### N-(((1S,9aS)-3-oxo-7-(6-(2-oxooxazolidin-3-yl)pyridin-3-yl)-1,3,9,9a-tetrahydrooxazolo[3,4-a]indol-1-yl)methyl)acetamide

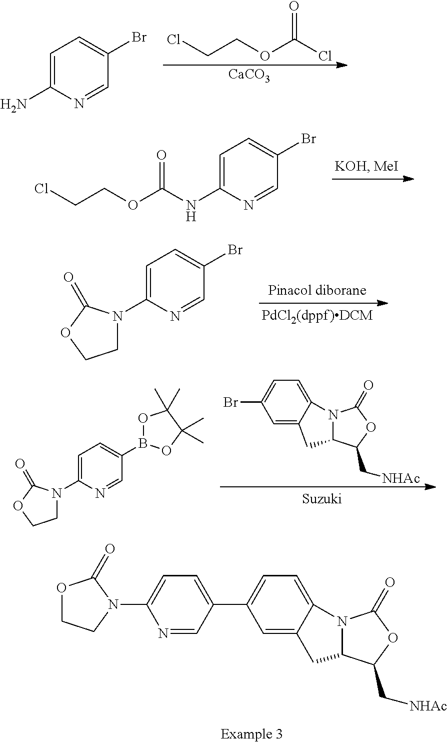

### Step 1. Preparation of 2-chloroethyl 5-bromopyridin-2-ylcarbamate

The mixture of 5-bromopyridin-2-amine (2.6 g, 15 mmol) and CaCO₃ (3.75 g, 37.5 mmol) in dioxane (30 mL) was heated to 70° C. and 2-chloroethyl carbonochloridate (5 mL) was added slowly over 30 min, stirred overnight at 70° C. Then the reaction was filtered while still hot and evaporated under vacuum to give a yellow solid. Recrystallized with MeOH/DCM (1:3) to give 2-chloroethyl 5-bromopyridin-2-ylcarbamate as a white solid (1.98 g, 47% yield).

### Step 2. Preparation of 3-(5-bromopyridin-2-yl)oxazolidin-2-one

To the solution of NH₃/MeOH (40 mL) in 200 mL autoclave was added 2-chloroethyl 5-bromopyridin-2-ylcarbamate (1.98 g, 7 mmol) and the mixture was heated to 120° C. for 3 h. Cooled to r.t., filtered and evaporated under vacuum to give a yellow solid, which was dissolved in 200 mL of DCM, treated with active carbon, passed through a sillica gel pad, and evaporated under vacuum to give 3-(5-bromopyridin-2-yl)oxazolidin-2-one a white solid (468 mg, 60% yield).

### Step 3. Preparation of 3-(5-(4,4,5,5-tetramethyl-1,3,2-dioxaborolan-2-yl)pyridin-2-yl)oxazolidin-2-one

To the solution of 3-(5-bromopyridin-2-yl)oxazolidin-2-one (486 mg, 2 mmol) in DMSO (10 mL) was added pinacol diborane (1.02 g, 4.0 mmol), KOAc (588 mg, 6 mmol) and PdCl₂dppfDCM (160 mg, 0.2 mmol), degassed and protected with N₂. The mixture was stirred at 80° C. for 2 h. The reaction mixture was diluted with DCM (100 mL) and washed with sat. aq. NaCl solution (100 mL×2), dried (Na₂SO₄) and evaporated under vacuum. The residue was purified by prep. TLC (hexanes—EtOAc) to give 3-(5-(4,4,5,5-tetramethyl-1,3,2-dioxaborolan-2-yl)pyridin-2-yl)oxazolidin-2-one as white solid (187 mg, 32% yield).

### Step 4. Preparation of N-(((1S,9aS)-3-oxo-7-(6-(2-oxooxazolidin-3-yl)pyridin-3-yl)-1,3,9,9a-tetrahydrooxazolo[3,4-a]indol-1-yl)methyl)acetamide

To the solution of 3-(5-(4,4,5,5-tetramethyl-1,3,2-dioxaborolan-2-yl)pyridin-2-yl)oxazolidin-2-one (133 mg, 0.46 mmol) in dioxane/H₂O (5:1, 5 mL) was added N-(((1S,9aS)-7-bromo-3-oxo-1,3,9,9a-tetrahydrooxazolo[3,4-a]indol-1-yl)methyl)acetamide (125 mg, 0.38 mmol), K₂CO₃ (158 mg, 1.14 mmol) and PdCl₂(dppf)DCM (30 mg, 0.04 mmol), degassed and protected with N₂. The mixture was stirred at 80° C. for 3 h. The reaction mixture was diluted with DCM/MeOH 2:1 (20 mL), filtered and evaporated under vacuum, then purified by preparative TLC to give N-(((1S,9aS)-3-oxo-7-(6-(2-oxooxazolidin-3-yl)pyridin-3-yl)-1,3,9,9a-tetrahydrooxazolo[3,4-a]indol-1-yl)methyl)acetamide as white solid (82 mg, 53% yield). ¹H NMR (DMSO-d₆, 300 MHz): 8.65-8.64 (m, 1H), 8.30-8.26 (m, 1H), 8.16-8.08 (m, 2H), 7.64-7.57 (m, 2H), 7.34-7.31 (m, 1H), 4.74-4.70 (m, 1H), 4.55-4.45 (m, 3H), 4.23-4.18 (m, 2H), 3.55-3.51 (m, 2H), 3.27-3.24 (m, 2H), 1.88 (s, 3H); MS (m/z): 409.1 [M+H]⁺.

### Example 4

### Preparation of N-(((1S,9aS)-7-(6-(1H-tetrazol-1-yl)pyridin-3-yl)-3-oxo-1,3,9,9a-tetrahydrooxazolo[3,4-a]indol-1-yl)methyl)acetamide

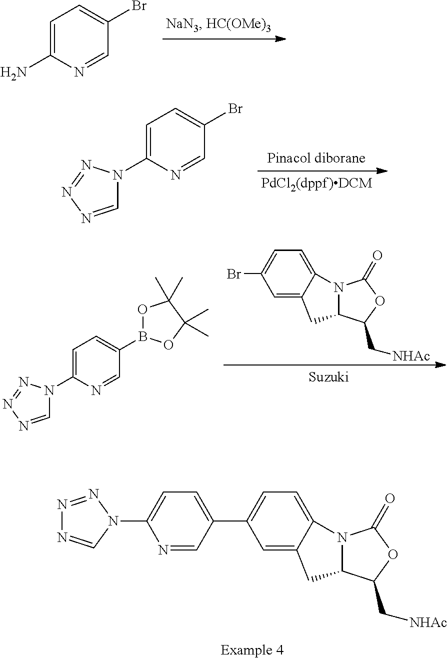

### Step 1. Preparation of 5-bromo-2-(1H-tetrazol-1-yl)pyridine

To the solution of 5-bromopyridin-2-amine (3.3 g, 19 mmol) in AcOH (14 mL) was added NaN₃ (1.43 g, 22 mmol) and HC(OMe)₃ (3.5 mL) and the mixture was warmed up to 80° C. for 5 h. Cooled to r.t., the reaction mixture was evaporated under vacuum and dissolved in water, extracted with EtOAc (100 mL×3), washed with 1N HCl (100 mL×2), sat. aq. NaHCO₃ (100 mL), brine (100 mL), and dried (Na₂SO₄). Solvent was evaporated to give a white solid. This was washed with EtOAc/hexanes (1:1) to afford 5-bromo-2-(1H-tetrazol-1-yl)pyridine as a white solid (3.69 g, 87% yield).

### Step 2. Preparation of 5-(4,4,5,5-tetramethyl-1,3,2-dioxaborolan-2-yl)-2-(1H-tetrazol-1-yl)pyridine

To the solution of 5-bromo-2-(1H-tetrazol-1-yl)pyridine (80 mg, 0.36 mmol) in dioxane (3 mL) was added pinacol diborane (100 mg, 0.39 mmol), KOAc (100 mg, 1.08 mmol) and PdCl2(dppf)DCM (10 mg, 0.01 mmol), degassed and protected with N₂. The mixture was stirred at 80° C. for 4 h. The reaction mixture was diluted with DCM (100 mL), filtered and evaporated under vacuum to give a yellow solid, purified by preparative TLC (solvent system here) to give 5-(4,4,5,5-tetramethyl-1,3,2-dioxaborolan-2-yl)-2-(1H-tetrazol-1-yl)pyridine as white solid (48 mg, 49% yield).

### Step 3. Preparation of N-(((1S,9aS)-7-(6-(1H-tetrazol-1-yl)pyridin-3-yl)-3-oxo-1,3,9,9a-tetrahydrooxazolo[3,4-a]indol-1-yl)methyl)acetamide

To the solution of 5-(4,4,5,5-tetramethyl-1,3,2-dioxaborolan-2-yl)-2-(1H-tetrazol-1-yl)pyridine (69 mg, 0.25 mmol) in dioxane/H₂O (5:1, 4 mL) was added N-(((1S,9aS)-7-bromo-3-oxo-1,3,9,9a-tetrahydrooxazolo[3,4-a]indol-1-yl)methyl)acetamide (128 mg, 0.25 mmol), K₂CO₃ (105 mg, 0.75 mmol) and PdCl₂(dppf)DCM (25 mg, 0.03 mmol), degassed and protected with N₂. The mixture was stirred at 80° C. for 3 h. The reaction mixture was diluted with DCM/MeOH 2:1 (20 mL), filtered, and evaporated under vacuum, then purified by preparative TLC (solvent system here) to give N-(((1S,9aS)-7-(6-(1H-tetrazol-1-yl)pyridin-3-yl)-3-oxo-1,3,9,9a-tetrahydrooxazolo[3,4-a]indol-1-yl)methyl)acetamide as white solid (82 mg, 57% yield). ¹H NMR (DMSO-d₆, 300 MHz): 10.20 (s, 1H), 8.93 (s, 1H), 8.45-8.41 (m, 1H), 8.29-8.27 (m, 1H), 8.13-8.10 (m, 1H), 7.78-7.71 (m, 2H), 7.40-7.37 (m, 1H), 4.77-4.73 (m, 1H), 4.61-4.52 (m, 1H), 3.56-3.50 (m, 2H), 3.30-3.27 (m, 2H), 1.88 (s, 3H); MS (m/z): 391.9 [M+H]⁺.

### Example 5

### Preparation of N-(((1S,9aS)-7-(2-methylpyrrolo[3,4-c]pyrazol-5(2H,4H,6H)-yl)-3-oxo-1,3,9,9a-tetrahydrooxazolo[3,4-a]indol-1-yl)methyl)acetamide

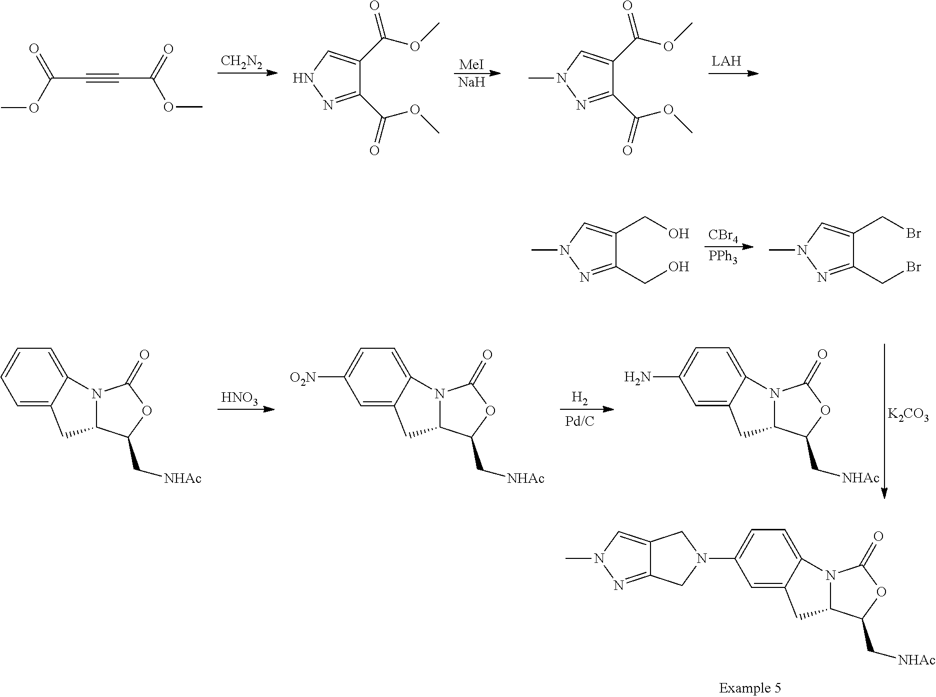

### Step 1. Preparation of dimethyl 1H-pyrazole-3,4-dicarboxylate

To the solution of diazomethane (˜218 mmol) in Et₂O (600 mL) was added dimethyl but-2-ynedioate (31 g, 218 mmol) dropwise over 1 h at 0° C. Then the mixture was filtered and washed with Et₂O to give dimethyl 1H-pyrazole-3,4-dicarboxylate as white crystals (17.4 g, 44% yield).

### Step 2. Preparation of dimethyl 1-methyl-1H-pyrazole-3,4-dicarboxylate

To the solution of dimethyl 1H-pyrazole-3,4-dicarboxylate (14.72 g, 80 mmol) in THF (500 mL) was added NaH (60%, 3.84 g, 96 mmol) and MeI (7.92 g, 55.8 mmol) at room temperature. The mixture was stirred for 3 h at room temperature. EtOAc was added followed by water, the mixture was extracted and the combined the organic phases were dried (MgSO₄, evaporated under vacuum, and the residue was recrystallized from hexanes gave dimethyl 1-methyl-1H-pyrazole-3,4-dicarboxylate as white solid (11.35 g, 72% yield).

### Step 3. Preparation of (1-methyl-1H-pyrazole-3,4-diyl)dimethanol

The solution of dimethyl 1-methyl-1H-pyrazole-3,4-dicarboxylate (4.0 g, 20 mmol) in anhydrous ether (10 mL) and anhydrous DCM (60 mL) was added slowly to a stirred suspension of LiAlH₄ (1.4 g, 37 mmol) in anhydrous ether (60 mL), and the mixture was reflux for 24 h. The solution was quenched by careful addition of MeOH, and the solvents were removed on a rotavap. DCM and MeOH were added to dissolve the mixture. The solution was filtered off, and the filtrate evaporated in vacuo. Crude (1-methyl-1H-pyrazole-3,4-diyl)dimethanol was purified by silica gel column chromatography (DCM:MeOH=20:1). Yield 2.5 g, 87%.

### Step 4. Preparation of 3,4-bis(bromomethyl)-1-methyl-1H-pyrazole

A three-neck flask (50 mL) was charged with (1-methyl-1H-pyrazole-3,4-diyl)dimethanol (284 mg, 2 mmol), PPh₃ (1.05 g, 4 mmol), CBr₄ (1.32 g, 4 mmol). Anhydrous DCM was added to the flask under N₂ atmosphere. After 30 min. stirring, another portion of PPh₃ (0.524 mg, 2 mmol) and CBr₄ (662 mg, 2 mmol) was added and the mixture was stirred for further 30 min. Then the solvent was removed and 3,4-bis(bromomethyl)-1-methyl-1H-pyrazole was obtained by silica gel column chromatography (petroleum: EtOAc=5:1). Yield 270 mg, 50%.

### Step 5. Preparation of N-(((1S,9aS)-7-amino-3-oxo-1,3,9,9a-tetrahydrooxazolo[3,4-a]indol-1-yl)methyl)acetamide

N-(((1S,9aS)-7-nitro-3-oxo-1,3,9,9a-tetrahydrooxazolo[3,4-a]indol-1-yl)methyl)acetamide (220 mg, 0.75 mmol) and 10% Pd/C (22 mg) in EtOH (5 mL) was stirred at r.t. for 4 h under H₂ (1 atm). The mixture was filtered through Celite, the filtrate evaporated under vacuum, and the product purified by preparative TLC (solvent system here) to give N-(((1S,9aS)-7-amino-3-oxo-1,3,9,9a-tetrahydrooxazolo[3,4-a]indol-1-yl)methyl)acetamide as white solid. Yield 169 mg, 85%. [α]25D−74.0° [C=0.25, THF/MeOH (1:1)]. MS (m/z): 262 [M+H]⁺.

### Step 6. Preparation of N-(((1S,9aS)-7-nitro-3-oxo-1,3,9,9a-tetrahydrooxazolo[3,4-a]indol-1-yl)methyl)acetamide

Fuming nitric acid (18 mL, 0.36 mol) was added dropwise with stirring to a solution of N-(((1S,9aS)-3-oxo-1,3,9,9a-tetrahydrooxazolo[3,4-a]indol-1-yl)methyl)acetamide (9 g, 0.036 mol; prepared as described for Example 1, Step 8) in AcOH (100 mL) at 0° C. The reaction mixture was stirred at r.t. for 2 h and poured into ice. The mixture was extracted by EtOAc (3×100 mL). The combined organic phase was dried (MgSO₄). Purification by chromatography (silica gel, EtOAc) afforded the title compound.

Yield 6.5 g, 61%.

### Step 7. Preparation of N-(((1S,9aS)-7-(2-methylpyrrolo[3,4-c]pyrazol-5(2H,4H,6H)-yl)-3-oxo-1,3,9,9a-tetrahydrooxazolo[3,4-a]indol-1-yl)methyl)acetamide

A 50 ml flask was charged with 3,4-bis(bromomethyl)-1-methyl-1H-pyrazole (230 mg, 0.86 mmol), N-(((1S,9aS)-7-amino-3-oxo-1,3,9,9a-tetrahydrooxazolo[3,4-a]indol-1-yl)methyl)acetamide (224 mg, 0.86 mmol), K₂CO₃ (608 mg, 4.29 mmol) and anhydrous DMF (40 mL). The mixture was heated to 60° C. and stirred for 1.5 h. Water and DCM were added, the organic layer separated, and the water phase was extracted twice with DCM. The combined the organic layers were washed with water 5 times, dried (Na₂SO₄) and N-(((1S,9aS)-7-(2-methylpyrrolo[3,4-c]pyrazol-5(2H,4H,6H)-yl)-3-oxo-1,3,9,9a-tetrahydrooxazolo[3,4-a]indol-1-yl)methyl)acetamide was purified by silica gel column chromatography (DCM:MeOH=20:1). Yield 105 mg, 33%. ¹H NMR (DMSO-d₆, 300 MHz): 8.27 (w, 1H), 7.58-7.51 (d, 1H), 7.06-6.86 (m, 3H), 4.63-4.61 (m, 1H), 4.47-4.24 (m, 4H), 4.09 (m, 1H), 3.74 (s, 3H), 3.49 (m, 2H), 3.10-3.01 (m, 2H), 1.86 (s, 3H); MS (m/z): 735 [2M+H]⁺.

### Example 6

### Preparation of N-(((1S,9aS)-3-oxo-7-(5H-pyrrolo[3,4-b]pyridin-6(7H)-yl)-1,3,9,9a-tetrahydrooxazolo[3,4-a]indol-1-yl)methyl)acetamide

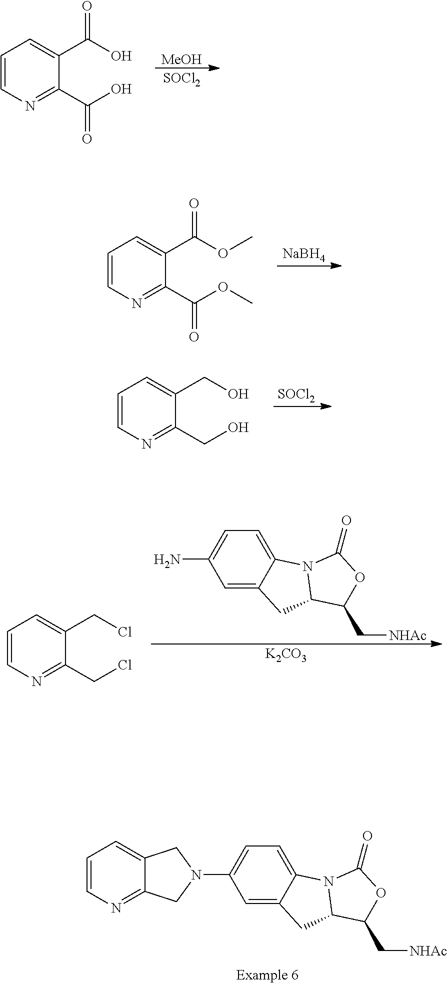

### Step 1. Preparation of dimethylpyridine-2,3-dicarboxylate

To the solution of pyridine-2,3-dicarboxylic acid (50 g, 300 mmol) in MeOH (300 mL) was added SOCl₂ (44 mL, 600 mmol), and the mixture was refluxed overnight. Volatiles were removed under vacuum, sat. Na₂CO₃ was added, and the mixture was extracted with EtOAc. The organic phase was washed with brine, dried (Na₂SO₄) and evaporated under vacuum to give dimethylpyridine-2,3-dicarboxylate as a colorless oil. Yield 32 g, 54%.

### Step 2. Preparation of pyridine-2,3-diyldimethanol

To the solution of dimethylpyridine-2,3-dicarboxylate (32 g, 164 mmol) in EtOH/H₂O (10:1, 440 mL) was added NaBH₄ (32 g, 842 mmol) portionwise over 30 min, and the mixture stirred at r.t. overnight. Acetone and EtOAc were added, and the mixture was filtered through a silica gel pad, and evaporated under vacuum to give a yellow solid. This was dissolved in EtOAc/MeOH (5:1), passed through a silica gel pad, and evaporated to give pyridine-2,3-diyldimethanol as a yellow solid. Yield 3.2 g, 14%.

### Step 3. Preparation of 2,3-bis(chloromethyl)pyridine

To the solution of pyridine-2,3-diyldimethanol (6 g, 43 mmol) in DCM (40 mL) was added SOCl₂ (32 mL, 430 mmol) and the mixture was refluxed overnight. The mixture was evaporated under vacuum to give a yellow semi-solid, dissolved in DCM and passed through a silica gel pad, evaporated under vacuum and purified by silica gel column to give 2,3-bis(chloromethyl)pyridine as a orange oil. Yield 1.8 g, 24%.

### Step 4. Preparation of N-(((1S,9aS)-3-oxo-7-(5H-pyrrolo[3,4-b]pyridin-6(7H)-yl)-1,3,9,9a-tetrahydrooxazolo[3,4-a]indol-1-yl)methyl)acetamide

A mixture of 2,3-bis(chloromethyl)pyridine (149 mg, 0.85 mmol), N-(((1S,9aS)-7-amino-3-oxo-1,3,9,9a-tetrahydrooxazolo[3,4-a]indol-1-yl)methyl)acetamide (200 mg, 0.76 mmol), K₂CO₃ (266 mg, 1.9 mmol), KI (27 mg, 0.16 mmol) and anhydrous DMF (10 mL) was stirred at 80^{˜}90° C. overnight under N₂. Water and DCM was then added to dilute the solution. The organic layer was separated and the water phase was extracted twice with DCM. The combined DCM solution was washed with water. The organic phase was dried (Na₂SO₄, evaporated under vacuum, and the product purified by preparative TLC (2-10% MeOHin DCM) to give N-(((1S,9aS)-3-oxo-7-(5H-pyrrolo[3,4-b]pyridin-6(7H)-yl)-1,3,9,9a-tetrahydrooxazolo[3,4-a]indol-1-yl)methyl)acetamide as white solid (145 mg, 80% yield). ¹H NMR (DMSO-d₆, 300 MHz): 8.50 (m, 1H), 7.64 (m, 1H), 7.35 (m, 1H), 7.21 (m, 1H), 6.55 (m, 2H), 6.09 (m, 1H), 4.56-4.63 (m, 5H), 4.47 (m, 1H), 3.79 (m, 1H), 3.66 (m, 1H), 3.14 (m, 1H), 3.22 (m, 1H), 2.06 (s, 3H); MS (m/z): 364.9 [M+1]⁺.

### Example 7

### Preparation of N-(((1S,9aS)-3-oxo-7-(6H-pyrrolo[3,4-b]pyridin-6-yl)-1,3,9,9a-tetrahydrooxazolo[3,4-a]indol-1-yl)methyl)acetamide

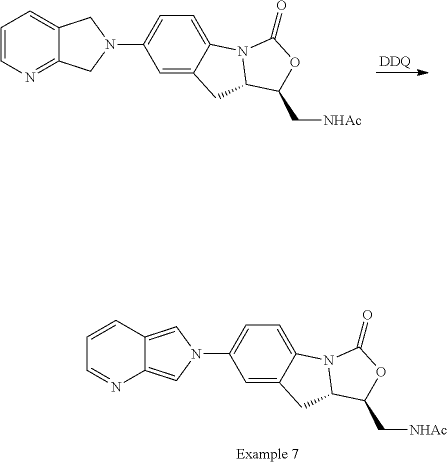

To the solution of N-(((1S,9aS)-3-oxo-7-(5H-pyrrolo[3,4-b]pyridin-6(7H)-yl)-1,3,9,9a-tetrahydrooxazolo[3,4-a]indol-1-yl)methyl)acetamide (68 mg, 0.19 mmol) in DCM/toluene (1:1, 20 mL) DDQ/toluene (43 mg, 0.20 mmol in 3 mL toluene) was added dropwise with stirring at 0° C. The mixture was allowed to warm up to r.t. over ca. 1-2 h. EtOAc (100 mL) was added, the organic mixture was washed with 5% aq. Na₂CO₃ (100 mL×2), sat. NaCl solution, and dried (Na₂SO₄). Solvent was evaporated under vacuum, and the crude product purified by preparation TLC (2-10% MeOHin DCM) to give N-(((1S,9aS)-3-oxo-7-(6H-pyrrolo[3,4-b]pyridin-6-yl)-1,3,9,9a-tetrahydrooxazolo[3,4-a]indol-1-yl)methyl)acetamide as white solid (47 mg, 68% yield). ¹H NMR (DMSO-d₆, 300 MHz): 8.46 (m, 1H), 7.95 (m, 1H), 7.68 (s, 1H), 7.54 (m, 1H), 7.46 (m, 2H), 7.35 (m, 1H), 6.90 (m, 1H), 6.04 (m, 1H), 4.59-4.67 (m, 2H), 3.80 (m, 1H), 3.74 (m, 1H), 3.38 (m, 1H), 3.24 (m, 1H), 2.07 (s, 3H); MS (m/z): 362.9 (M+1).

### Example 8

### Preparation of N-(((1S,9aS)-7-(6-(5-(hydroxymethyl)-4,5-dihydro isoxazol-3-yl)pyridin-3-yl)-3-oxo-1,3,9,9a-tetrahydrooxazolo[3,4-a]indol-1-yl)methyl)acetamide

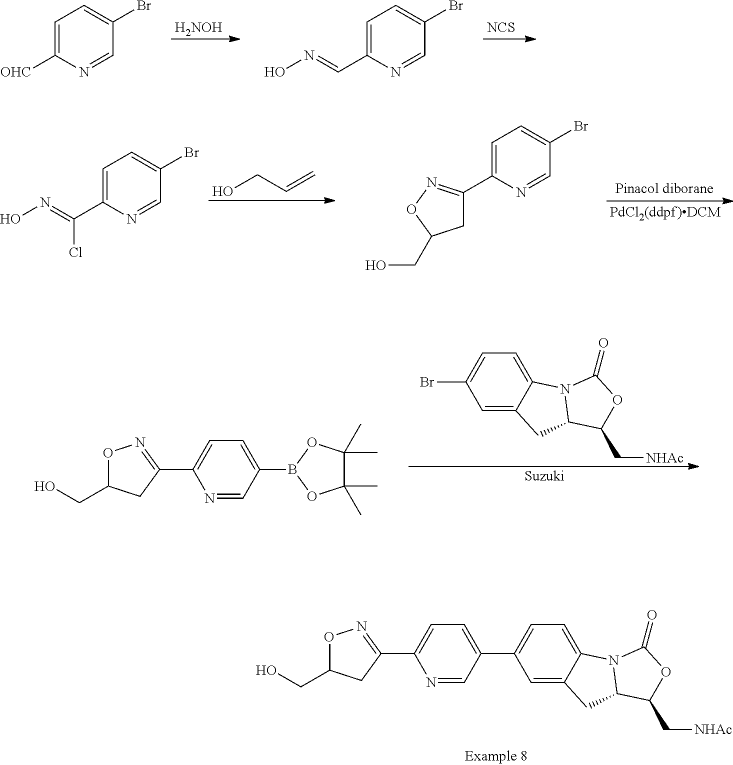

### Step 1. Preparation of 5-bromopicolinaldehyde oxime

To a solution of 5-bromopicolinaldehyde (7 g, 38 mmol) in methanol (100 mL) and water (90 mL) was added NH₂OH.HCl (3.4 g, 49=01). Then, Na₂CO₃ (2.7 g, 25 mmol) in 10 mL of water was added. The reaction mixture was stirred at r.t. for 30 min. Water (30 mL) was added, the precipitate filtered off and washed with water to give 5-bromopicolinaldehyde oxime (7 g, 93% yield), which was used for the next step without further purification.

### Step 2. Preparation of (3-(5-bromopyridin-2-yl)-4,5-dihydroisoxazol-5-yl)methanol

To a solution of 5-bromopicolinaldehyde oxime (1 g, 5 mmol) in 30 mL of anhydrous DMF was added N-chlorosuccinimide (0.8 g, 6 mmol; NCS) at 60° C. under N2 atmosphere. The reaction mixture was stirred at 60° C. for 30 min. The reaction mixture was cooled to 0° C. and prop-2-en-1-ol (1.5 g, 25 mmol) was added. The reaction mixture was stirred at 0° C. for 10 min. A mixture of Et₃N (0.7 g, 7 mmol) in 5 mL of anhydrous DMF was added dropwise. The reaction mixture was stirred at 0° C. for 30 min and then stirred at r.t. for 1 h. EtOAc was added to the reaction mixture. The solution was washed with water for several times. The organic phase was dried (Na₂SO₄) and evaporated under vacuum. The residue was purified by silca gel column chromatography to give (3-(5-bromopyridin-2-yl)-4,5-dihydroisoxazol-5-yl)methanol (1.1 g, 85% yield).

### Step 3. Preparation of (3-(5-(4,4,5,5-tetramethyl-1,3,2-dioxaborolan-2-yl)pyridin-2-yl)-4,5-dihydroisoxazol-5-yl)methanol

(3-(5-Bromopyridin-2-yl)-4,5-dihydroisoxazol-5-yl)methanol (1.5 g, 5.86 mmol), pinacol diborane (4.5 g, 18 mmol) and KOAc (1.7 g, 18 mmol) were added to 30 mL of anhydrous dioxane. The slurry was degassed with N2 for 3 min. Then 200 mg of PdCl₂(dppf)DCM was added. The reaction mixture was refluxed for 2 h, cooled to r.t., and filtered. The filtrate was evaporated under vacuum under reduced pressure. The residue was dissolved in EtOAc and washed with water. The aq. phase was extracted with EtOAc. The combined organic phases were dried (Na2SO4 and evaporated under vacuum. The crude product was purified by silica gel column to give (3-(5-(4,4,5,5-tetramethyl-1,3,2-dioxaborolan-2-yl)pyridin-2-yl)-4,5-dihydroisoxazol-5-yl)methanol (880 mg: 34% yield).

### Step 4. Preparation of N-(((1S,9aS)-7-(6-(5-(hydroxymethyl)-4,5-dihydroisoxazol-3-yl)pyridin-3-yl)-3-oxo-1,3,9,9a-tetrahydrooxazolo[3,4-a]indol-1-yl)methyl)acetamide

(3-(5-(4,4,5,5-Tetramethyl-1,3,2-dioxaborolan-2-yl)pyridin-2-yl)-4,5-dihydroisoxazol-5-yl)methanol (350 mg, 1.15 mmol), N-(((1S,9aS)-7-bromo-3-oxo-1,3,9,9a-tetrahydrooxazolo[3,4-a]indol-1-yl)methyl)acetamide (90 mg, 0.28 mmol) and K₂CO₃ (130 mg, 0.94 mmol) were added to dioxane/water(5:1, 6 mL). The slurry was degassed with N2 for 3 min. Then 20 mg of Pd Cl₂(dppf)DCM was added. The reaction mixture was stirred at 90° C. for 3 h. The solvent was removed under vacuum, residue was dissolved in EtOAc, and washed with water. The aqueous phase was extracted with EtOAc. The combined organic phases were dried (Na₂SO₄ and evaporated under vacuum. The crude product was purified by silica gel column (solvent system here) to give N-(((1S,9aS)-7-(6-(5-(hydroxymethyl)-4,5-dihydroisoxazol-3-yl)pyridin-3-yl)-3-oxo-1,3,9,9a-tetrahydrooxazolo[3,4-a]indol-1-yl)methyl)acetamide as a white solid (75 mg, 64% yield). ¹H NMR (DMSO-d₆, 300 MHz): 8.92 (dd, 1H), 8.31 (t, 1H), 8.12 (dd, 1H), 7.95 (dd, 1H), 7.73 (s, 1H), 7.67 (dd, 1H), 7.36 (d, 1H), 5.00-5.03 (m, 1H), 4.73-4.78 (m, 2H), 4.53-4.56 (m, 1H), 3.51-3.56 (m, 4H), 3.25-3.41 (m, 4H), 1.88 (s, 3H); MS (m/z): 423.1 (M+1).

### Example 9

### Preparation of N-(((1S,9aS)-7-(5-fluoro-6-(5-(hydroxymethyl)-4,5-dihydroisoxazol-3-yl)pyridin-3-yl)-3-oxo-1,3,9,9a-tetrahydrooxazolo[3,4-a]indol-1-yl)methyl)acetamide

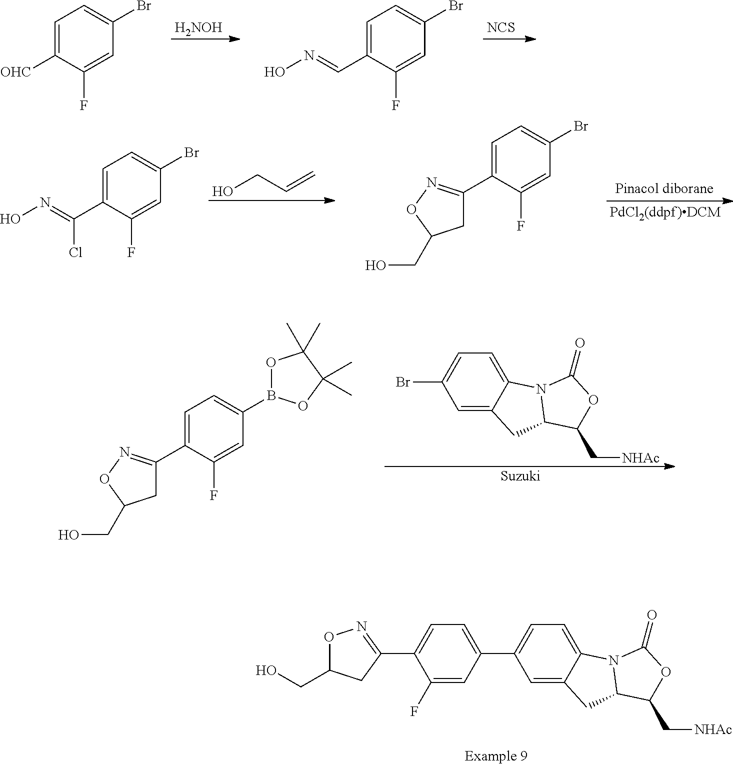

### Step 1. Preparation of 5-bromo-3-fluoropicolinaldehyde oxime

5-Bromo-3-fluoropicolinaldehyde (6.0 g, 29.4 mmol) was dissolved in 120 mL MeOH with 60 mL H₂O. NH₂OH.HCl (3.0 g, 43.2 mmol) and Na₂CO₃ (3.0 g, 28.3 mmol) were added, and the mixture stirred at r.t. for 1.5 h. MeOH was removed in vacuo, and the mixture was extracted with EtOAc. The organic layer washed with brine, dried Na₂SO₄ and evaporated under vacuum to give the crude 5-bromo-3-fluoropicolinaldehyde oxime (6.0 g, yield 94%).

### Step 2. Preparation of (3-(5-bromo-3-fluoropyridin-2-yl)-4,5-dihydroisoxazol-5-yl)methanol

To the solution of 5-bromo-3-fluoropicolinaldehyde oxime (2.9 g, 13.4 mmol) in anhydrous DMF (50 mL) was added NCS (1.97 g, 14.74 mmol), under N2 atmosphere heated to 60° C. The mixture was stirred at 60° C. for 0.5 h. Then the reaction mixture was cooled to 2° C. and the allyl alcohol (3.89 g, 67.0 mmol) was added. The mixture of Et₃N (1.49 g, 14.74 mmol) in 10 mL DMF was added dropwise to the reaction mixture over 30 min, stirred 30 min at 2° C. and then at r.t. for 1 h. The solvent was removed under vacuum, the residue was dissolved in EtOAc and washed with sat. aq. Na₂CO₃ and brine. The EtOAc layers were dried (Na₂SO₄) and evaporated in vacuo. The residue was purified by column chromatography (hexanes—EtOAc 4/1) to give (3-(5-bromo-3-fluoropyridin-2-yl)-4,5-dihydroisoxazol-5-yl)methanol (3.24 g, 86% yield).

### Step 3. Preparation of (3-(3-fluoro-5-(4,4,5,5-tetramethyl-1,3,2-dioxaborolan-2-yl)pyridin-2-yl)-4,5-dihydroisoxazol-5-yl)methanol

To the solution of (3-(5-bromo-3-fluoropyridin-2-yl)-4,5-dihydroisoxazol-5-yl)methanol (500 mg, 1.82 mmol) in anhydrous dioxane (20 mL) was added pinacol diborane (927 mg, 3.65 mmol), KOAc (536 mg, 5.47 mmol) and PdCl₂dppfDCM (100 mg, 0.12 mmol), degassed and protected with N₂. The mixture was stirred at 80° C. for 2 h. The reaction mixture was evaporated to dryness, and the product isolated by prep. TLC (1% MeOH in DCM) to give (3-(3-fluoro-5-(4,4,5,5-tetramethyl-1,3,2-dioxaborolan-2-yl)pyridin-2-yl)-4,5-dihydroisoxazol-5-yl)methanol (517 mg, yield 88%).

### Step 4. Preparation of N4(1S,9aS)-7-(5-fluoro-6-(5-(hydroxymethyl)-4,5-dihydroisoxazol-3-yl)pyridin-3-yl)-3-oxo-1,3,9,9a-tetrahydrooxazolo[3,4-a]indol-1-yl)methyl)acetamide

To the solution of (3-(3-fluoro-5-(4,4,5,5-tetramethyl-1,3,2-dioxaborolan-2-yl)pyridin-2-yl)-4,5-dihydroisoxazol-5-yl)methanol (138 mg, 0.43 mmol) in dioxane/H₂O (5/1, 12 mL) was added N-(((1S,9aS)-7-bromo-3-oxo-1,3,9,9a-tetrahydrooxazolo[3,4-a]indol-1-yl)methyl)acetamide (100 mg, 0.31 mmol; prepared as described for Example 1, Step 9), K₂CO₃ (128 mg, 0.92 mmol) and PdCl₂dppfDCM (26 mg, 0.031 mmol). The mixture was degassed and stirred at 90° C. for 3 h under N2. Then the mixture was diluted with EtOAc (150 mL), washed with water, brine, and dried (Na₂SO₄). Solvent was evaporated under vacuum, and the residue purified by prep-TLC (1% MeOH in DCM) to give N-(((1S,9aS)-7-(5-fluoro-6-(5-(hydroxymethyl)-4,5-dihydroisoxazol-3-yl)pyridin-3-yl)-3-oxo-1,3,9,9a-tetrahydrooxazolo[3,4-a]indol-1-yl)methyl)acetamide (74 mg, yield 55%). ¹H NMR (DMSO-d₆, 300 MHz): 8.31-8.27 (m, 1H), 7.80-7.56 (m, 5H), 7.34-7.31 (m, 1H), 5.01-4.97 (m, 1H), 4.76-4.68 (m, 2H), 4.55-4.53 (m, 1H), 3.55-3.41 (m, 5H), 3.24-3.21 (m, 3H), 1.88 (s, 3H); MS (m/z): 440.2 [M+1]⁺.

### Example 10

### Preparation of N-(((1S,9aS)-7-(6-(5-(morpholinomethyl)-4,5-dihydroisoxazol-3-yl)pyridin-3-yl)-3-oxo-1,3,9,9a-tetrahydrooxazolo[3,4-a]indol-1-yl)methyl)acetamide

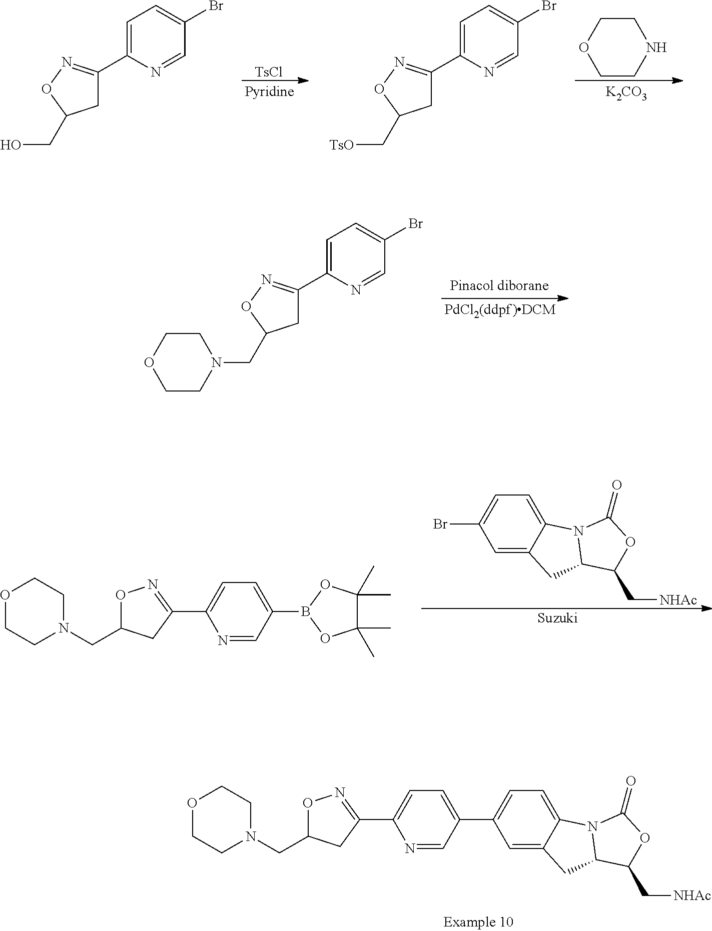

### Step 1. Preparation of (3-(5-bromopyridin-2-yl)-4,5-dihydroisoxazol-5-yl)methyl 4-methylbenzenesulfonate

TsCl (1.1 g, 5.8 mmol) was added to a solution of (3-(5-bromopyridin-2-yl)-4,5-dihydroisoxazol-5-yl)methanol (1.0 g, 2.9 mmol) in 20 mL of anhydrous Py under N2. The reaction mixture was stirred at r.t. overnight, then cooled to 0° C. Sat. NaHCO₃ was added dropwise, and the solvent was removed under vacuum. The residue was dissolved in EtOAc and washed with water, brine, and dried (Na₂SO₄). Solvent was evaporated under vacuum. The crude product was purified by column chromatography on silica gel (hexanes—EtOAc) to give compound (3-(5-bromopyridin-2-yl)-4,5-dihydroisoxazol-5-yl)methyl 4-methylbenzenesulfonate (1.14 g, 96% yield).

### Step 2. Preparation of 44(3-(5-bromopyridin-2-yl)-4,5-dihydroisoxazol-5-yl)methyl)morpholine

(3-(5-Bromopyridin-2-yl)-4,5-dihydroisoxazol-5-yl)methyl 4-methylbenzenesulfonate (1.14 g, 2.8 mmol) was added to the mixture of K₂CO₃(0.8 g, 5.7 mmol) in 20 mL of DMSO. Then morpholine (2.5 g, 28 mmol) was added dropwise. The reaction mixture was stirred at 90° C. for 2 h. EtOAc was added, and the solution was washed with water, brine, and dried (Na₂SO₄). The crude product was purified by column chromatography on silica gel (solvent system here) to give 4-((3-(5-bromopyridin-2-yl)-4,5-dihydroisoxazol-5-yl)methyl)morpholine (0.55 g, 61% yield).

### Step 3. Preparation of 4-((3-(5-(4,4,5,5-tetramethyl-1,3,2-dioxaborolan-2-yl)pyridin-2-yl)-4,5-dihydroisoxazol-5-yl)methyl)morpholine

44(3-(5-Bromopyridin-2-yl)-4,5-dihydroisoxazol-5-yl)methyl)morpholine (500 mg, 1.53 mmol), bis(pinacolate)diborone (470 mg, 1.84) and KOAc (300 mg, 3.1 mmol) were added to 20 mL of anhydrous dioxane. The slurry was degassed with N2 for 3 min. Then 50 mg of PdCl₂(dppf)DCM was added. The reaction mixture was stirred at 100° C. for 2 h, cooled to r.t., and filtered. The filtrate was evaporated under vacuum, and the residue dissolved in EtOAc and washed with water. The aqueous phase was extracted with EtOAc. The combined organic phase was dried (Na₂SO₄) and evaporated under vacuum. The crude product was purified by column chromatography on silica gel (EtOAc—hexanes) to give 4-((3-(5-(4,4,5,5-tetramethyl-1,3,2-dioxaborolan-2-yl)pyridin-2-yl)-4,5-dihydroisoxazol-5-yl)methyl)morpholine (200 mg, 35% yield).

### Step 4. Preparation of N-(((1S,9aS)-7-(6-(5-(morpholinomethyl)-4,5-dihydroisoxazol-3-yl)pyridin-3-yl)-3-oxo-1,3,9,9a-tetrahydrooxazolo[3,4-a]indol-1-yl)methyl)acetamide

4-((3-(5-(4,4,5,5-Tetramethyl-1,3,2-dioxaborolan-2-yl)pyridin-2-yl)-4,5-dihydroisoxazol-5-yl)methyl)morpholine (200 mg), N-(((1S,9aS)-7-bromo-3-oxo-1,3,9,9a-tetrahydrooxazolo[3,4-a]indol-1-yl)methyl)acetamide (60 mg, 0.18 mmol) and K₂CO₃ (83 mg, 0.6 mmol) were added to 5 mL of dioxane and 1 mL of water. The slurry was degassed with N2, and 10 mg of PdCl₂(dppf)DCM was added. The reaction mixture was stirred at 90° C. for 3 h. The solvent was removed under vacuum. The residue was dissolved in EtOAc and washed with water. The aqueous phase was extracted with EtOAc. The combined organic phase was dried (Na₂SO₄ and evaporated under vacuum. The crude product was purified by column chromatography on silica gel (solvent system here) to give N-(((1S,9aS)-7-(6-(5-(morpholinomethyl)-4,5-dihydroisoxazol-3-yl)pyridin-3-yl)-3-oxo-1,3,9,9a-tetrahydrooxazolo[3,4-a]indol-1-yl)methyl)acetamide (60 mg, 68% yield). ¹H NMR (DMSO-d₆, 300 MHz): 8.92 (dd, 1H), 8.31 (t, 1H), 8.12 (dd, 1H), 7.95 (dd, 1H), 7.72 (s, 1H), 7.67 (dd, 1H), 7.37 (d, 1H), 4.90-5.00 (m, 1H), 4.73-4.76 (m, 1H), 4.51-4.59 (m, 1H), 3.49-3.59 (m, 7H), 3.18-3.28 (m, 5H), 2.55-2.58 (m, 2H), 2.45-2.51 (m, 2H), 1.88 (s, 3H); MS (m/z): 492.2 [M+1]⁺.

### Example 11

### Preparation of N-(((1S,9aS)-7-(6-(2-(1H-imidazol-1-yl)acetyl)pyridin-3-yl)-3-oxo-1,3,9,9a-tetrahydrooxazolo[3,4-a]indol-1-yl)methyl)acetamide

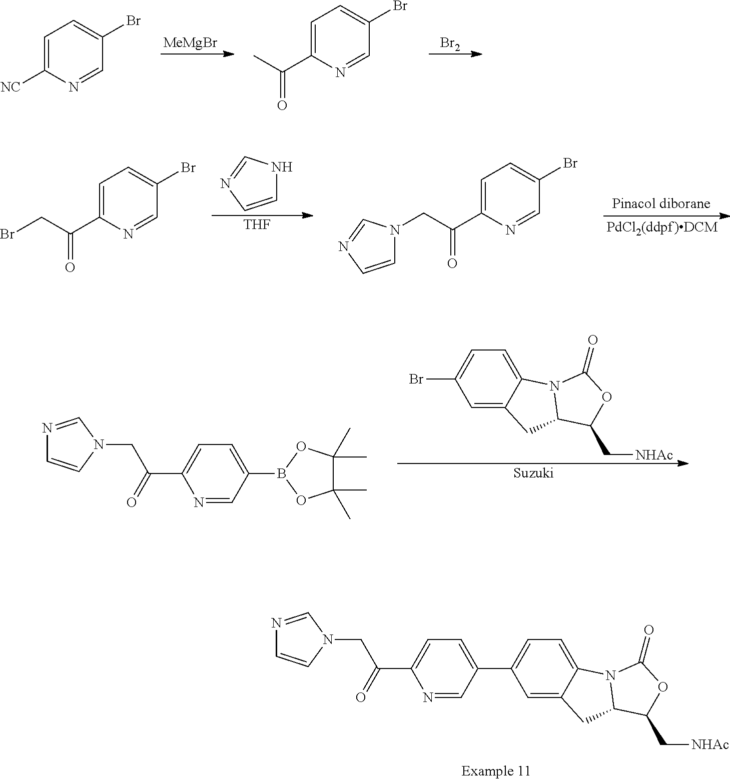

### Step 1. Preparation of 1-(5-bromonyridin-2-yflethanone

5-Bromopicolinonitrile (5.4 g, 29.7 mmol) was dissolved in anhydrous THF (120 mL) and cooled to −20° C. MeMgBr (35 mL, 1M in Et₂O) was added dropwise, and the reaction mixture was stirred at −20° C. for 3 h. The reaction mixture was cooled to −40° C., and neutralized with conc. HCl. The solvent was removed under reduced pressure. The residue was dissolved in EtOAc and washed with water. The aqueous layer was extracted with EtOAc. The combined organic phase was dried (Na₂SO₄) and evaporated under vacuum. The crude product was purified by column chromatography on silica gel (EtOAc—hexanes) to give 1-(5-bromopyridin-2-yl)ethanone (1.9 g, 30% yield).

### Step 2. Preparation of 2-bromo-1-(5-bromonyridin-2-yflethanone

Br₂ (1.2 g, 7.6 mmol) was added to a solution of 1-(5-bromopyridin-2-yl)ethanone (1.5 g, 7.6 mmol) in 60 mL of CCl₄. The reaction mixture was sealed and stirred at 70° C. overnight. The solvent was removed under reduced pressure. The residue was dissolved in EtOAc and washed with water. The aqueous was extracted with EtOAc for several times. The combined organic phases were dried (Na₂SO₄ and evaporated under vacuum. The crude product was purified by column chromatography on silica gel (EtOAc—hexanes) to give compound 2-bromo-1-(5-bromopyridin-2-yl)ethanone (1.2 g, 57% yield).

### Step 3. Preparation of 1-(5-bromopyridin-2-yl)-2-(1H-imidazol-1-yl)ethanone

2-Bromo-1-(5-bromopyridin-2-yl)ethanone (1.2 g, 4.3 mmol) was added to a solution of imidazole (2.9 g, 43 mmol) in 30 mL of THF. The reaction mixture was stirred at r.t. overnight. The solvent was removed under reduced pressure. The residue was dissolved in EtOAc and washed with water. The aqueous phase was extracted with EtOAc. The combined organic phase was dried (Na₂SO₄) and evaporated under vacuum. The crude product was purified by column chromatography on silica gel (EtOAc—hexanes) to give compound 1-(5-bromopyridin-2-yl)-2-(1H-imidazol-1-yl)ethanone (540 mg, 47% yield).

### Step 4. Preparation of N-(((1S,9aS)-3-oxo-7-(4,4,5,5-tetramethyl-1,3,2-dioxaborolan-2-yl)-1,3,9,9a-tetrahydrooxazolo[3,4-a]indol-1-yl)methyl)acetamide

4,4,5,5-Tetramethyl-2-(4,4,5,5-tetramethyl-1,3,2-dioxaborolan-2-yl)-1,3,2-dioxaborolane (470 mg, 1.85 mmol) was added to a solution of N-(((1S,9aS)-7-bromo-3-oxo-1,3,9,9a-tetrahydrooxazolo[3,4-a]indol-1-yl)methyl)acetamide (500 mg, 1.54 mmol, 1.00 equiv) in 1,4-dioxane (9 mL) under N₂, followed by PdCl₂(dppf)DCM (77 mg, 0.14 mmol) and KOAc (302 mg, 3.08 mmol). The mixture was stirred at 80° C. for 48 h, then cooled to r.t. EtOAc (300 mL) was added, and the mixture was filtered. The filtrate was evaporated under vacuum, and the residue purified by flash chromatography (eluent: EtOAc—hexanes 3:1). This yielded 472 mg (83%) of N-(((1S,9aS)-3-oxo-7-(4,4,5,5-tetramethyl-1,3,2-dioxaborolan-2-yl)-1,3,9,9a-tetrahydrooxazolo[3,4-a]indol-1-yl)methyl)acetamide as a light yellow solid. ¹H NMR (CDCl₃, 300 MHz): 7.77 (d, 1H), 7.69 (s, 1H), 7.46 (d, 1H), 5.92 (s, 2H), 4.60-3.00 (m, 4H), 2.01 (s, 3H), 1.29 (s, 9H). MS (m/z): 373 [M+H]⁺.

### Step 5. Preparation of N-(((1S,9aS)-7-(6-(2-(1H-imidazol-1-yl)acetyl)pyridin-3-yl)-3-oxo-1,3,9,9a-tetrahydrooxazolo[3,4-a]indol-1-yl)methyl)acetamide

N-(((1S,9aS)-3-oxo-7-(4,4,5,5-tetramethyl-1,3,2-dioxaborolan-2-yl)-1,3,9,9a-tetrahydrooxazolo[3,4-a]indol-1-yl)methyl)acetamide (200 mg, 0.54 mmol), 1-(5-bromopyridin-2-yl)-2-(1H-imidazol-1-yl)ethanone (200 mg, 0.76 mmol) and K₂CO₃ (150 mg, 1.12 mmol) were added to 5 mL of dioxane and 1 mL of water. The slurry was degassed under N₂, then 30 mg of PdCl₂(dppf)DCM was added. The reaction mixture was stirred at 80° C. overnight. The solvent was removed under vacuum, and the residue was dissolved in EtOAc, and washed with water. The aqueous phase was extracted with EtOAc. The combined organic phase was dried (Na₂SO₄) and evaporated under vacuum. The crude product was purified by column chromatography on silica gel (2-10% MeOHin DCM) to give N-(((1S,9aS)-7-(6-(2-(1H-imidazol-1-yl)acetyl)pyridin-3-yl)-3-oxo-1,3,9,9a-tetrahydrooxazolo[3,4-a]indol-1-yl)methyl)acetamide (66 mg, 20.1% yield). ¹H NMR (DMSO-d₆, 300 MHz): 8.87 (dd, 1H), 8.14 (m, 1H), 8.01 (m, 1H), 7.56 (m, 3H), 7.49 (m, 1H), 7.15 (s, 1H), 7.00 (m, 1H), 5.91-5.99 (m, 1H), 5.68 (s, 2H), 4.58-4.66 (m, 2H), 3.70-3.86 (m, 2H), 3.36-3.46 (m, 1H), 3.18-2.25 (m, 1H), 2.07 (s, 3H); MS (m/z): 431.8 [M+1]⁺.

### Example 12

### Preparation of N-(((1S,9aS)-7-(6-(5,5-bis(hydroxymethyl)-4,5-dihydroisoxazol-3-yl)pyridin-3-yl)-3-oxo-1,3,9,9a-tetrahydrooxazolo[3,4-a]indol-1-yl)methyl)acetamide

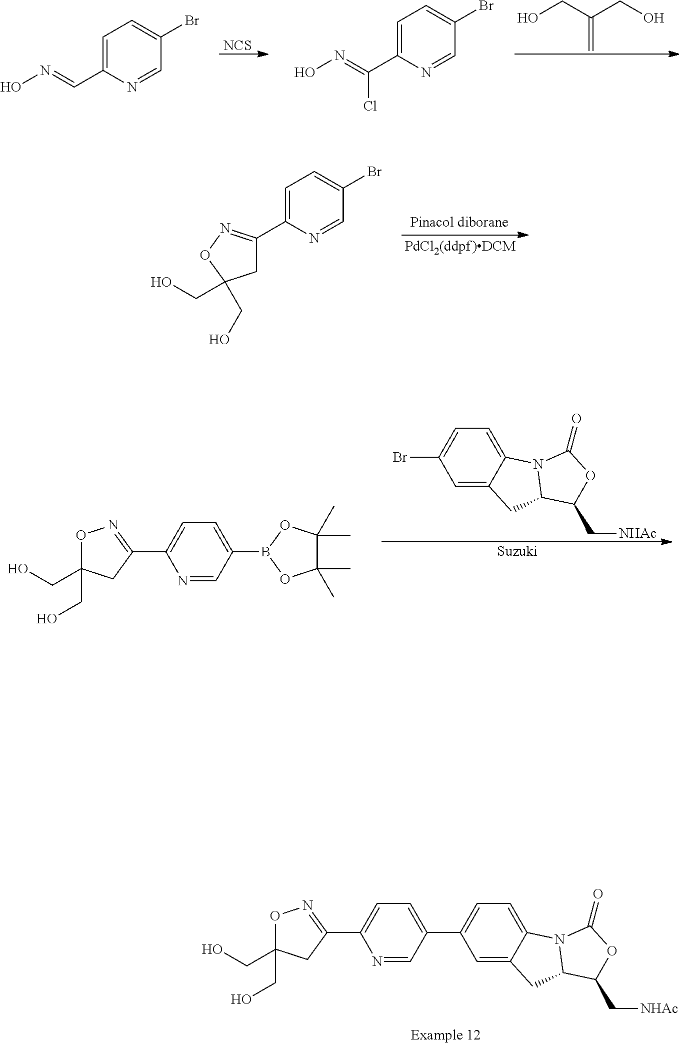

### Step 1. Preparation of (3-(5-bromopyridin-2-yl)-4,5-dihydroisoxazole-4,4-diyl)dimethanol

To the solution of 5-bromopicolinaldehyde oxime (1.02 g, 5.1 mmol, prepared as for Example 8) in anhydrous DMF (25 mL) was added NCS (812 mg, 6.1 mmol) under a N₂ atmosphere. The mixture was stirred at 60° C. for 1 h, then cooled to 0° C., and 2-methylenepropane-1,3-diol (2.23 g, 25 mmol) was added. Et₃N (717 mg, 7 mmol) in 5 mL DMF was added dropwise over 30 min, and the mixture was stirred 30 min at 0° C., and then at r.t. for 2 h. The solvent was removed under vacuum, and the residue was dissolved in EtOAc and washed with water. Aq. layer were extracted with EtOAc, the combined EtOAc layers were dried (Na₂SO₄) and evaporated under vacuum. The residue was purified by column chromatography (hexanes—EtOAc 2/1) to give (3-(5-bromopyridin-2-yl)-4,5-dihydroisoxazole-4,4-diyl)dimethanol (1.02 g, yield 70%).

### Step 2. Preparation of N-(((1S,9aS)-7-(6-(5,5-bis(hydroxymethyl)-4,5-dihydroisoxazol-3-yl)pyridin-3-yl)-3-oxo-1,3,9,9a-tetrahydrooxazolo[3,4-a]indol-1-yl)methyl)acetamide

To the solution of (3-(5-bromopyridin-2-yl)-4,5-dihydroisoxazole-4,4-diyl)dimethanol (208 mg, 0.73 mmol) in dioxane/H₂O (5/1, 12 mL) was added N-(((1S,9aS)-3-oxo-7-(4,4,5,5-tetramethyl-1,3,2-dioxaborolan-2-yl)-1,3,9,9a-tetrahydrooxazolo[3,4-a]indol-1-yl)methyl)acetamide (180 mg, 0.48 mmol), K₂CO₃ (200 mg, 1.45 mmol) and PdCl₂dppfDCM (81 mg, 0.097 mmol). The mixture was degassed with N₂ and stirred at 90° C. for 5 h. Then the mixture was diluted with EA, washed with water and brine, dried (Na₂SO₄ and evaporated under vacuum in vacuo. The residue was purified by prep-TLC (DCM/MeOH=10/1) to give N-(((1S,9aS)-7-(6-(5,5-bis(hydroxymethyl)-4,5-dihydroisoxazol-3-yl)pyridin-3-yl)-3-oxo-1,3,9,9a-tetrahydrooxazolo[3,4-a]indol-1-yl)methyl)acetamide (70 mg, yield 32%). ¹H NMR (DMSO-d₆, 300 MHz): 8.93 (m, 1H), 8.29 (t, 1H), 8.11 (m, 1H), 7.94 (d, 1H), 7.72 (s, 1H), 7.67 (d, 1H), 7.35 (d, 1H), 5.01 (t, 2H), 4.74 (m, 1H), 4.56 (q, 1H), 3.51-3.56 (m, 6H), 3.29 (s, 2H), 3.22-3.25 (m, 2H), 1.88 (s, 3H); MS (m/z): 453.1 [M+1]⁺.

### Example 13

### Preparation of N-(((1S,9aS)-7-(3-methoxyazetidin-1-yl)-3-oxo-1,3,9,9a-tetrahydrooxazolo[3,4-a]indol-1-yl)methyl)acetamide

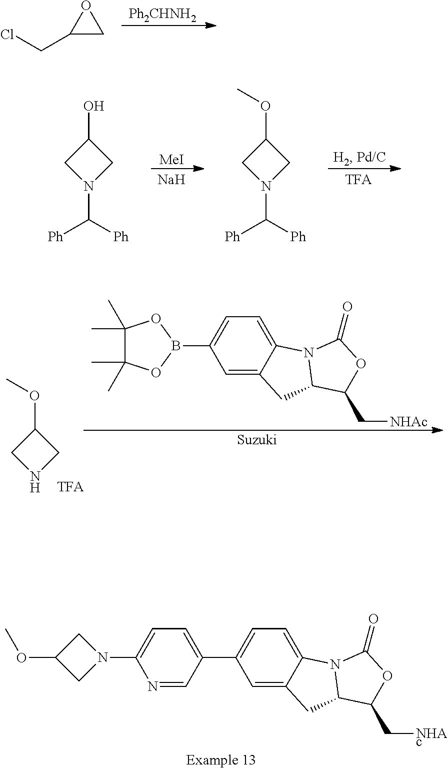

### Step 1. Preparation of 1-benzhydrylazetidin-3-ol

The solution of 2-(chloromethyl)oxirane (10 mL, 128 mmol) and Ph₂CHNH₂ (11 mL, 64 mmol) in DMF (80 mL) was heated at 90-100° C. under N₂ for 3 d. The mixture was cooled to r.t. and evaporated under vacuum to give a yellow oil, which was dissolved in DCM (200 mL), washed with water (200 mL×2), and extracted with 1N HCl (200 mL×2). The aq. solution was made basic with 10% aq. NaOH, the mixture was extracted with Et₂O (200 mL×3), dried (Na₂SO₄), and evaporated under vacuum to give 1-benzhydrylazetidin-3-ol (5.74 g, 38% yield).

### Step 2. Preparation of 1-benzhydryl-3-methoxyazetidine

To the solution of 1-benzhydrylazetidin-3-ol (5.74 g, 24 mmol) in DMF (120 mL) was added NaH (60%, 1.92 g, 48 mmol) portionwise with stirring over 20 min at 0° C. under N₂, then MeI (6.8 g, 48 mmol) was added, and the solution was warmed up to r.t. and stirred for 1 h. The mixture was poured into brine, extracted with Et₂O (200 mL×3), and dried (Na₂SO₄). Solvent was evaporated under vacuum, and the crude material purified by silica gel column chromatography (hexanes—EtOAc) to give 1-benzhydryl-3-methoxyazetidine as colorless oil (5.37 g, 80% yield).

### Step 3. Preparation of 3-methoxyazetidine

To the solution of 1-benzhydryl-3-methoxyazetidine (2.53 mg, 10 mmol) in EtOH (100 mL) was added TFA (1.25 g, 11 mmol) and 10% Pd/C (1.6 g), and the mixture was stirred at r.t. for 2 h under 1 atm of H₂, then filtered through Celite and evaporated under vacuum to give 3-methoxyazetidine as a white solid (2 g, quant.).

### Step 4. Preparation of N-(((1S,9aS)-7-(3-methoxyazetidin-1-yl)-3-oxo-1,3,9,9a-tetrahydrooxazolo[3,4-a]indol-1-yl)methyl)acetamide

N-(((1S,9aS)-3-oxo-7-(4,4,5,5-tetramethyl-1,3,2-dioxaborolan-2-yl)-1,3,9,9a-tetrahydrooxazolo[3,4-a]indol-1-yl)methyl)acetamide (190 mg, 0.5 mmol), Cu(OAc)₂.H₂O (100 mg, 0.5 mmol), KF (35 mg, 0.6 mmol), 4A molecule sieves (1.0 g) and MeCN (12 mL) were placed in microwave reaction tube, the mixture was stirred and purged with O₂ for 10 min. A mixture of 3-methoxyazetidine (500 mg, 2.5 mmol), Et₃N (350 mg, 3.5 mmol) in MeCN (3 mL) was added dropwise, and the mixture was stirred and saturated with O₂ for another 10 min. The tube was sealed, placed in the microwave reactor, and the latter was then operated at 100° C. for 5 h. The mixture was filtered, and the filtrate was evaporated under vacuum to give a brown semi-solid that was purified by prep-TLC (5% MeOH in DCM) to give N-(((1S,9aS)-7-(3-methoxyazetidin-1-yl)-3-oxo-1,3,9,9a-tetrahydrooxazolo[3,4-a]indol-1-yl)methyl)acetamide as a yellow solid (67 mg, 40% yield). ¹H NMR (CDCl₃, 300 MHz): 7.24 (m, 1H), 6.33 (m, 2H), 6.11 (m, 1H), 4.55 (m, 1H), 4.43 (m, 1H), 4.31 (m, 1H), 4.06 (m, 2H), 3.78 (m, 1H), 3.55-3.67 (m, 3H), 3.33 (s, 3H), 3.31 (m, 1H), 3.09 (m, 1H), 2.04 (s, 3H); MS (m/z): 332.1 [M+1⁺].

### Example 14

### Preparation of tert-butyl 4-((1S,9aS)-1-(acetamidomethyl)-3-oxo-1,3,9,9a-tetrahydrooxazolo[3,4-a]indol-7-yl)-5,6-dihydropyridine-1(2H)-carboxylate

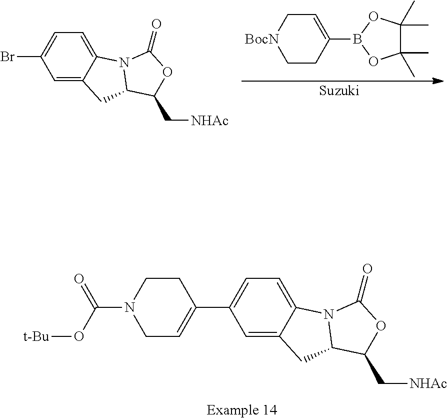

1,2-Dimethoxyethane (5 mL) was added to tert-butyl 4-(4,4,5,5-tetramethyl-1,3,2-dioxaborolan-2-yl)-5,6-dihydropyridine-1(2H)-carboxylate (210 mg, 0.69 mmol; prepared as per Wustrow et al. Synthesis, 1991, p. 993), N-(((1S,9aS)-7-bromo-3-oxo-1,3,9,9a-tetrahydrooxazolo[3,4-a]indol-1-yl)methyl)acetamide (150 mg, 0.46 mmol; prepared as for Example 1, Step 9), PdCl₂(dppf) DCM (34 mg 0.046 mmol), followed by 2M aqueous Na₂CO₃ (1 mL). The reaction mixture was heated at 80° C. overnight, cooled to r.t., and filtered through a short silica gel pad. After washing several times with EtOAc, the filtrate was evaporated under vacuum, and the residue was purified by column chromatography (EtOAc). The product was obtained as an off-white solid (80 mg, 26%). ¹H NMR (CDCl₃, 300 MHz): 7.35 (1H), 7.24 (2H), 6.43 (1H), 5.97 (1H), 4.62 (1H), 4.53 (1H), 4.07 (2H), 3.79 (1H), 3.70 (1H), 3.63 (2H), 3.28 (1H), 3.14 (1H), 2.49 (2H), 2.07 (3H), 1.50 (9H); MS (m/z)=428 [M+H].

### Example 15

### Preparation of N-(((1S,9aS)-7-(1-(2-acetoxyacetyl)-1,2,3,6-tetrahydropyridin-4-yl)-3-oxo-1,3,9,9a-tetrahydrooxazolo[3,4-a]indol-1-yl)methyl)acetamide

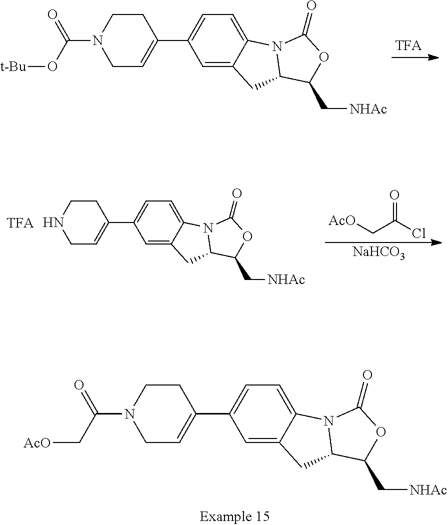

### Step 1. Preparation of N-(((1S,9aS)-3-oxo-7-(1,2,3,6-tetrahydropyridin-4-yl)-1,3,9,9a-tetrahydrooxazolo[3,4-a]indol-1-yl)methyl)acetamide trifluoroacetate

A solution of tert-butyl 4-((1S,9aS)-1-(acetamidomethyl)-3-oxo-1,3,9,9a-tetrahydrooxazolo[3,4-a]indol-7-yl)-5,6-dihydropyridine-1(2H)-carboxylate (18 mg, 0.04 mmol) in 0.5 mL of DCE was cooled to 0° C. To this mixture was added 0.5 mL of 20% TFA in 1,2-dichloroethane (DCE). The reaction mixture was stirred for 30 min at 0° C., then condensed under vacuum. More DCE was added, and the solvent was removed again under vacuum. The process was repeated several times to afford the product as a light-brownish solid. MS (m/z)=−328 [M+H]⁺.

### Step 2. Preparation of N-(((1S,9aS)-7-(1-(2-acetoxyacetyl)-1,2,3,6-tetrahydropyridin-4-yl)-3-oxo-1,3,9,9a-tetrahydrooxazolo[3,4-a]indol-1-yl)methyl)acetamide

The above Boc-deprotection product was dissolved in 1 mL of acetone and 1 mL of aqueous NaHCO₃ (0.2 M). To the reaction mixture was added acetoxyacetyl chloride (7 uL, 0.06 mmol). The reaction mixture was stirred for 1 h at r.t., then quenched with MeOH. The mixture was extracted with EtOAc (5 mL×2). The combined organic layers were washed with brine, and dried (Na₂SO₄). The filtrate was evaporated under vacuum, and the residue was purified by preparative TLC (10% MeOH in DCM). The product was obtained as a solid (16 mg, 92%). ¹H NMR (CDCl₃, 300 MHz) 7.37 (1H), 7.24 (2H), 6.22 (1H), 6.02 (1H), 4.82 (2H), 4.63 (1H), 4.54 (1H), 4.25 (2H), 3.83 (2H), 3.71 (1H), 3.61 (1H), 3.30 (1H), 3.13 (1H), 2.60 (2H), 2.22 (3H), 2.08 (3H). MS (m/z)=428.

### Example 16

### Preparation of N-(((1S,9aS)-7-(1-(2-hydroxyacetyl)-1,2,3,6-tetrahydropyridin-4-yl)-3-oxo-1,3,9,9a-tetrahydrooxazolo[3,4-a]indol-1-yl)methyl)acetamide

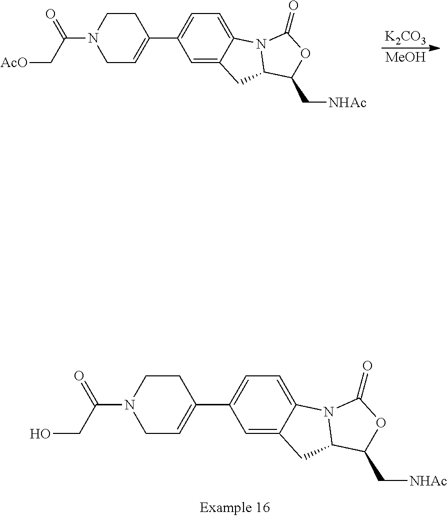

Compound of Example 15 (17.5 mg, 0.041 mmol) and K₂CO₃ (10 mg, 0.072 mmol) in MeOH (1 ml) and THF (0.5 ml) was stirred at r.t. for 2 h, and concentrated under reduced pressure. After redissolving in DCM, the solution was purified by preparative TLC eluting with 5% MeOH/DCM to afford the title compound. MS: (m/z) 386.0 [M+H]⁺.

### Example 17

### Preparation of (1R,5S,6s)-tert-butyl 6-(5-((1S,9aS)-1-(acetamidomethyl)-3-oxo-1,3,9,9a-tetrahydrooxazolo[3,4-a]indol-7-yl)pyridin-2-yl)-6-cyano-3-azabicyclo[3.1.0]hexane-3-carboxylate

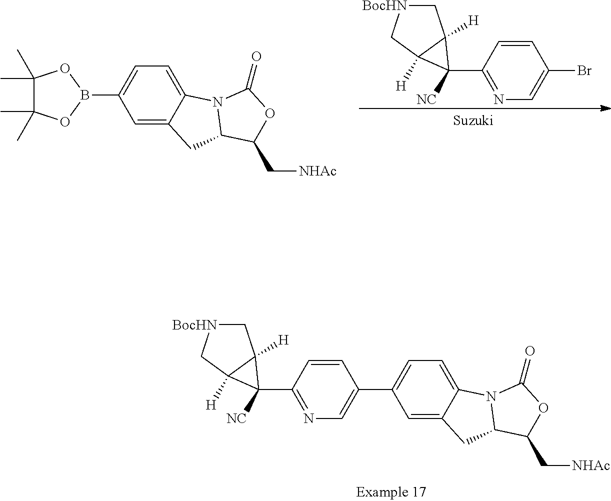

N-(((1S,9aS)-3-oxo-7-(4,4,5,5-tetramethyl-1,3,2-dioxaborolan-2-yl)-1,3,9,9a-tetrahydrooxazolo[3,4-a]indol-1-yl)methyl)acetamide (472 mg, 1.27 mmol, 1.00 equiv; prepared as described for Example 11, Step 4) was added to a solution of (1S,5R)-tert-butyl 6-(5-bromopyridin-2-yl)-6-cyano-3-aza-bicyclo[3.1.0]hexane-3-carboxylate (446 mg, 1.22 mmol; prepared as described in WO 2005/005398) in degassed dioxane (13 mL) under N₂, followed by Pd(PPh₃)₄ (147 mg, 0.13 mmol, 0.10 equiv) was added, followed by a solution of Na₂CO₃ (673 mg, 8.11 mmol, 6.39 equiv) in water (3.7 mL). The resulting solution was stirred for 3 h at 80° C. The resulting solution was diluted with 300 mL of EtOAc, and the mixture was washed brine, and dried (MgSO₄). Solvent was evaporated in vacuo, and the residue was purified by silica gel column chromatography with a 20:1 DCM/MeOH solvent system. This afforded 450 mg (67%) of the product isolated as a white solid. ¹H NMR: (CDCl₃): δ 8.65 (d, 1H), 7.86-7.82 (m, 2H), 7.54-7.42 (m, 3H), 4.72 (d, 1H), 5.99 (s, 2H), 3.61 (d, 2H), 3.43 (s, 1H), 3.34-3.24 (m, 6H), 2.70 (s, 1H), 2.01 (s, 3H), 1.29 (s, 9H). MS (m/z): 530 [M+H]⁺.

### Example 18

### Preparation of N-(((1S,9aS)-7-(6-((1R,5S,6s)-6-cyano-3-azabicyclo[3.1.0]hexan-6-yl)pyridin-3-yl)-3-oxo-1,3,9,9a-tetrahydrooxazolo[3,4-a]indol-1-yl)methyl)acetamide

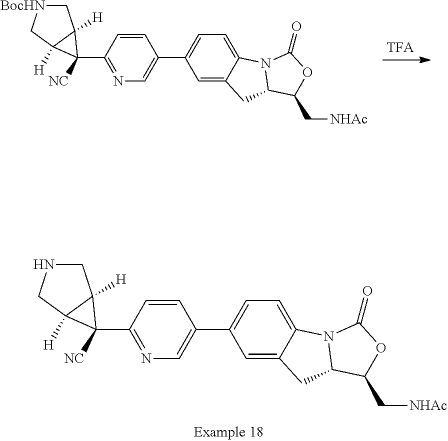

Trifluoroacetic acid (1 mL) was added dropwise with stirring at 4° C. to a solution of (1S,5R)-tert-butyl 6-(5-((1S,9aS)-1-(acetamidomethyl)-3-oxo-1,3,9,9a-tetrahydrooxazolo[3,4-a]indol-7-yl)pyridin-2-yl)-6-cyano-3-aza-bicyclo[3.1.0]hexane-3-carboxylate (400 mg, 0.76 mmol, 1.00 equiv) in DCM (2 mL). The mixture was kept at 4° C. for 2 h. Volatiles were removed under vacuum, and the residue was taken in 5 mL of DCM and 1 mL of MeOH. It was then evaporated under vacuum to dryness after adjusting the pH value to 7 with a saturated aq. NaHCO₃ solution. The residue was purified by flash chromatography with a 20:1 DCM/MeOH solvent system. This resulted in 181 mg (56%) of the product isolated as a white solid. ¹H NMR (CD₃OD): δ 8.71 (d, 1H), 7.98-8.02 (m, 1H), 7.65 (d, 1H), 7.54 (d, 2H), 7.42 (d, 1H), 4.72 (d, 1H), 4.59 (d, 1H), 3.61 (d, 2H), 3.43 (s, 1H), 3.34-3.24 (m, 6H), 2.70 (s, 1H), 2.01 (s, 3H). MS (m/z): 430 [M+H]⁺

### Example 19

### Preparation of N-(((1S,9aS)-7-(6-((1R,5S)-3,6-dicyano-3-azabicyclo[3.1.0]hexan-6-yl)pyridin-3-yl)-3-oxo-1,3,9,9a-tetrahydrooxazolo indol-1-yl)methyl)acetamide

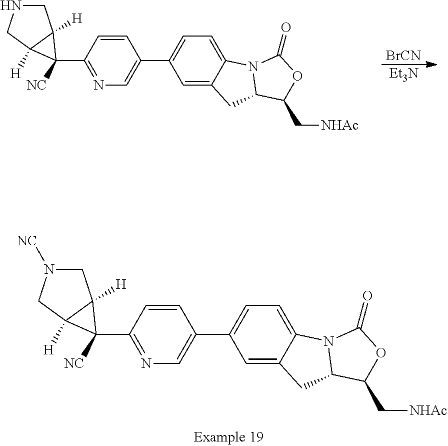

Cyanogen bromide (15 mg, 0.14 mmol) in DCM (1 ml) was added with stirring to a solution of N-(((1S,9aS)-7-(6-((1R,5S,6s)-6-cyano-3-azabicyclo[3.1.0]hexan-6-yl)pyridin-3-yl)-3-oxo-1,3,9,9a-tetrahydrooxazolo[3,4-a]indol-1-yl)methyl)acetamide 20 mg (0.046 mmol; Example 18) in MeOH (1 ml) at ca. 0° C. The reaction was allowed to warm up to r.t. and stirred overnight. Volatiles were removed under vacuum, and the product was purified by preparative TLC eluting with 5% MeOH/DCM to afford the title compound (17.0 mg). NMR (300 MHz, CDCl₃): δ 8.39 (m, 1H), 8.04 (t, 1H), 7.66 (dd, J1=8.10 Hz, J2=2.40, 1H), 7.52 (d, J=8.40 Hz, 1H), 7.24-7.21 (m, 3H), 4.46 (m, 1H), 4.33 (m, 1H), 3.83 (m, 2H), 3.60 (m, 2H), 3.44 (m, 2H), 3.19-2.98 (m, 2H), 2.61 (m, 2H), 1.81 (s, 3H). MS: (m/z) 455.1 [M+H]⁺.

### Example 20

### Preparation of (1R,5S)-methyl 6-(5-((1S,9aS)-1-(acetamidomethyl)-3-oxo-1,3,9,9a-tetrahydrooxazolo[3,4-a]indol-7-yl)pyridin-2-yl)-6-cyano-3-azabicyclo[3.1.0]hexane-3-carboxylate

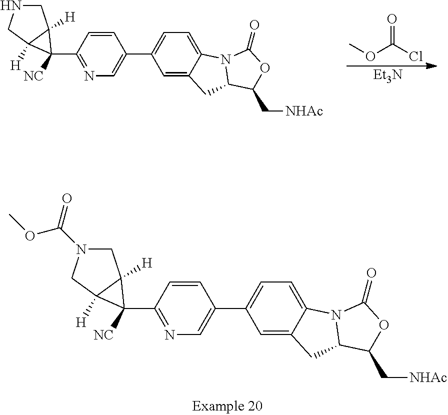

A solution of methyl chloroformate (4 μl, 0.051 mmol) in DCM (0.5 ml) was added with stirring at ca. 0° C. to a suspension of N-(((1S,9aS)-7-(6-((1R,5S,6s)-6-cyano-3-azabicyclo[3.1.0]hexan-6-yl)pyridin-3-yl)-3-oxo-1,3,9,9a-tetrahydrooxazolo[3,4-a]indol-1-yl)methyl)acetamide (20 mg, 0.046 mmol; Example 18) in DCM (0.8 ml) and Et₃N (20 μl, 0.14 mmol), and the mixture was stirred at r.t. for 3 h. Volatiles were removed under vacuum, and the product was purified by preparative TLC eluting with 5% MeOH/DCM to afford (1R,5S)-methyl 6-(5-((1S,9aS)-1-(acetamidomethyl)-3-oxo-1,3,9,9a-tetrahydrooxazolo[3,4-a]indol-7-yl)pyridin-2-yl)-6-cyano-3-azabicyclo[3.1.0]hexane-3-carboxylate (19.6 mg). ¹H NMR (300 MHz, CDCl₃): δ 8.47 (m, 1H), 7.89 (t, J=6.0 Hz, 1H), 7.72 (dd, J1=8.10 Hz, J2=2.40, 1H), 7.58 (d, J=8.10 Hz, 1H), 7.33-7.23 (m, 3H), 4.50 (m, 1H), 4.41 (m, 1H), 3.84˜3.77 (m, 2H), 3.73˜3.68 (m, 2H), 3.59 (3, 3H), 3.53 (m, 2H), 3.22-3.06 (m, 2H), 2.64 (m, 2H), 1.89 (s, 3H). MS: (m/z) 488.1 [M+H]⁺.

### Example 21

### Preparation of N-(((1S,9aS)-7-(6-((1R,5S)-6-cyano-3-(2-acetoxyacetyl)-3-azabicyclo[3.1.0]hexan-6-yl]pyridin-3-yl)-3-oxo-1,3,9,9a-tetrahydrooxazolo[3,4-a]indol-1-yl)methyl)acetamide

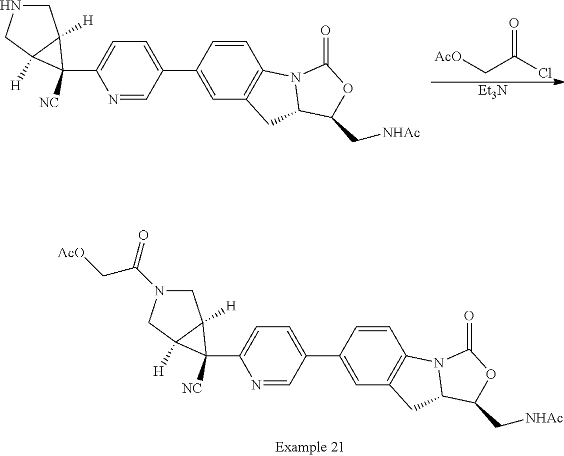

Example 21 was prepared analogously to Example 20, except that acetoxyacetyl chloride was used in place of methyl chloroformate.

¹H NMR (300 MHz, CD₃OD): δ 8.59 (m, 1H), 7.86 (dd, J1=8.40 Hz, J2=2.40, 1H), 7.68 (d, J=8.10 Hz, 1H), 7.42-7.40 (m, 3H), 4.75 (m, 1H), 4.63 (m, 1H), 4.11˜4.02 (m, 2H), 3.90˜3.85 (m, 2H), 3.66˜3.63 (m, 2H), 3.37˜3.31 (m, 3H), 3.18 (m, 1H), 2.91˜2.80 (m, 2H), 2.16 (s, 3H), 2.01 (s, 3H). MS: (m/z) 530.0 [M+H]⁺.

### Example 22

### Preparation of N-(((1S,9aS)-7-(6-((1R,5S)-6-cyano-3-(2-hydroxyacetyl)-3-azabicyclo[3.1.0]hexan-6-yl)pyridin-3-yl)-3-oxo-1,3,9,9a-tetrahydrooxazolo[3,4-a]indol-1-yl)methyl)acetamide

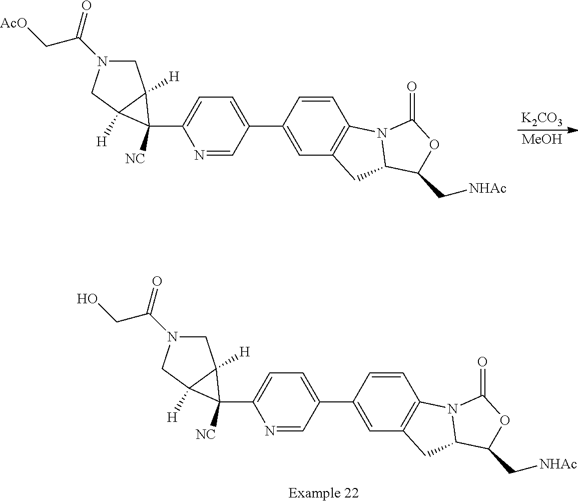

To a solution of compound of Example 21 (20 mg, 0.038 mmol) in MeOH (3 ml) and THF (1 ml) was added K₂CO₃ (10 mg, 0.072 mmol). The mixture was left at r.t. for 2 h, and concentrated under reduced pressure. After redissolved in DCM, the solution was purified on a preparative TLC eluting with 5% MeOH/DCM to afford the title compound. ¹H NMR (300 MHz, CDCl₃): δ 8.45 (m, 1H), 7.71˜7.68 (m, 1H), 7.53 (d, J=8.10 Hz, 1H), 7.28-7.24 (m, 3H), 4.50 (m, 1H), 4.41 (m, 1H), 3.81˜3.72 (m, 2H), 3.68˜3.49 (m, 2H), 3.17 (m, 2H), 3.07 (m, 1H), 2.75-2.65 (m, 2H), 1.87 (s, 3H). MS: (m/z) 488.1 [M+H]⁺.

### Example 23

### Preparation of N-(((1S,9aS)-7-(6-((1R,5S)-6-cyano-3-formyl-3-azabicyclo[3.1.0]hexan-6-yl)pyridin-3-yl)-3-oxo-1,3,9,9a-tetrahydrooxazolo[3,4-a]indol-1-yl)methyl)acetamide

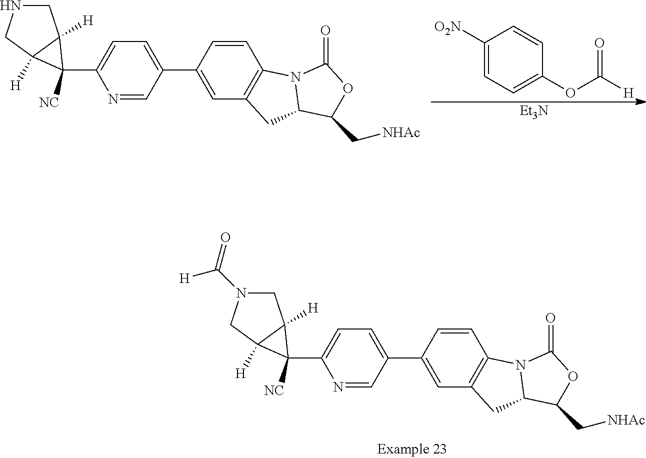

To a solution of compound of Example 18 (20 mg, 0.047 mmol) in THF (1 ml) was added p-nitrophenyl formate (9.3 mg, 0.055 mmol). The mixture was heated to 60° C. for 4 h, and concentrated under reduced pressure. The crude product was purified by preparative TLC eluting with 5% MeOH/DCM to afford the title compound. ¹H NMR (300 MHz, CD₃OD): 8.71 (s, 1H), 8.17 (s, 1H), 8.01 (m, 1H), 7.65 (dd, J1=8.40 Hz, 1H), 7.54 (m, 2H), 7.40 (d, J=8.10 Hz, 1H), 4.72 (m, 1H), 4.61 (m, 1H), 4.11 (s, 2H), 4.04 (m, 1H), 3.72˜3.66 (m, 3H), 3.37 (m, 1H), 3.25 (m, 1H), 2.84 (s, 2H), 2.02 (s, 3H). MS (m/z): 458.2 [M+H]⁺.

### Example 24

### Preparation of 3-((1S,9aS)-1-(Acetamidomethyl)-3-oxo-1,3,9,9a-tetrahydrooxazolo[3,4-a]indol-7-yl)pyridine 1-oxide

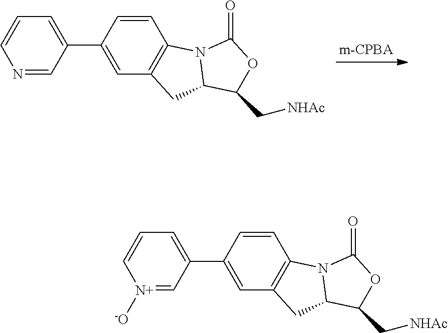

A solution of N-(((1S,9aS)-3-oxo-7-(pyridin-3-yl)-1,3,9,9a-tetrahydrooxazolo[3,4-a]indol-1-yl)methyl)acetamide (17 mg, 0.18 μmol) in DCM (1.2 ml) was cooled on ice-water bath, and then m-chloroperbenzoic acid was added (17 mg, ca 4.9 μmol). The reaction mixture was left at r.t. overnight, and the product purified by a preparative TLC with 10% MeOH/DCM. ¹H NMR (CDCl₃, 300 MHz): 8.56 (b, 1H), 8.28 (b, 1H), 7.85 (d, 1H, J=7.50 Hz), 7.59-7.41 (m, 3H), 4.74 (m, 1H), 4.58 (m, 1H), 3.64 (m, 2H), 3.25 (m, 2H), 2.02 (s, 3H). MS (m/z): 340.1 [M+H]⁺.

### Example 25

### Preparation of N-(((1S,9aS)-3-oxo-7-(1,4-dioxa-8-azaspiro[4.5]decan-8-yl)-1,3,9,9a-tetrahydrooxazolo[3,4-a]indol-1-yl)methyl)acetamide

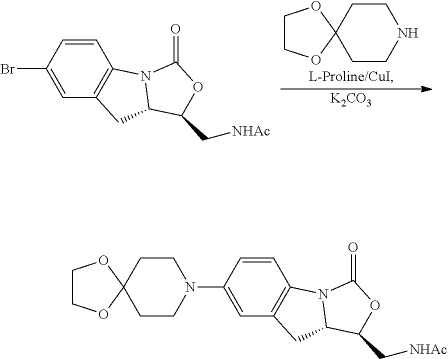

A mixture of N-(((1S,9aS)-7-bromo-3-oxo-1,3,9,9a-tetrahydrooxazolo[3,4-a]indol-1-yl)methyl)acetamide (80 mg, 0.25 μmol), 1,4-dioxa-8-azaspiro[4.5]decane (48 μl, 0.37 μmol), L-proline (35 mg), K₂CO₃ (220 mg), CuI (99) mg in DMSO (3 ml) was degassed with N₂, stirred at r.t. for 1 h, and then heated to 70° C. overnight. It was then quenched with water, and the mixture was extracted with EtOAc. Upon drying (MgSO₄) and solvent removal, the product was purified by silica gel chromatography using 5% MeOH/DCM to afford the title compound. ¹H NMR (DMSO-d₆, 300 MHz): 8.93 (m, 1H), 8.29 (t, 1H), 8.11 (m, 1H), 7.94 (d, 1H), 7.72 (s, 1H), 7.67 (d, 1H), 7.35 (d, 1H), 5.01 (t, 2H), 4.74 (m, 1H), 4.56 (q, 1H), 3.51-3.56 (m, 6H), 3.29 (s, 2H), 3.22-3.25 (m, 2H), 1.88 (s, 3H). MS (m/z): 388.1 [M+H]⁺.

### Examples 26 and 27

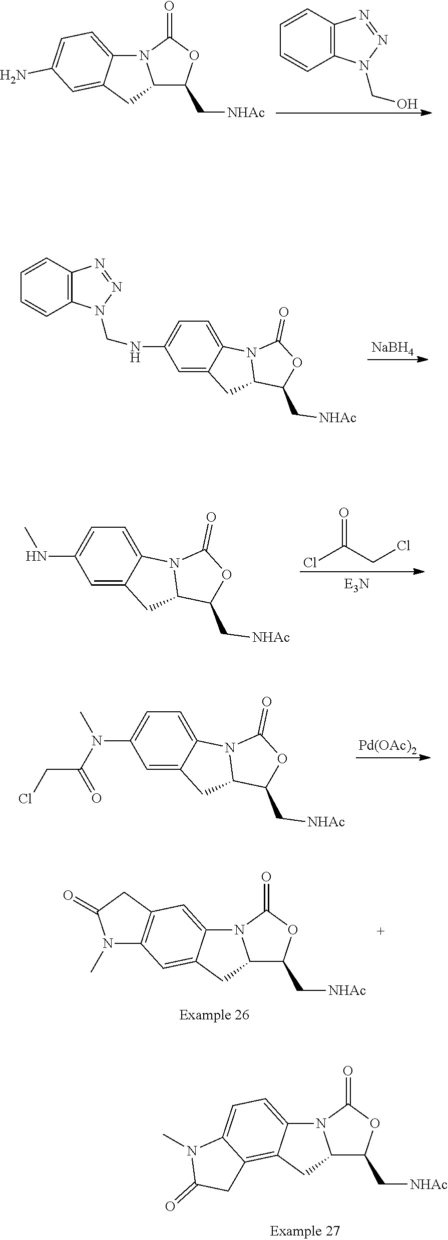

### Step 1. Preparation of N-(((1S,9aS)-7-((1H-benzo[d][1,2,3]triazol-1-yl)methylamino)-3-oxo-1,3,9,9a-tetrahydrooxazolo[3,4-a]indol-1-yl)methyl)acetamide

The suspension of N-(((1S,9aS)-7-amino-3-oxo-1,3,9,9a-tetrahydrooxazolo[3,4-a]indol-1-yl)methyl)acetamide 360 mg (1.38 mmol, prepared as for Example 5), and (1H-benzo[d][1,2,3]triazol-1-yl)methanol 230 mg (1.54 mmol) in EtOH (5 ml) was heated to dissolution, and briefly brought to a reflux (ca. 5 sec). The mixture was the stirred at r.t. overnight. The precipitated product was filtered and isolated as a white solid that was used directly in the next step.

### Step 2. Preparation of N-(((1S,9aS)-7-(methylamino)-3-oxo-1,3,9,9a-tetrahydrooxazolo[3,4-a]indol-1-yl)methyl)acetamide

To a solution of N-(((1S,9aS)-7-((1H-benzo[d][1,2,3]triazol-1-yl)methylamino)-3-oxo-1,3,9,9a-tetrahydrooxazolo[3,4-a]indol-1-yl)methyl)acetamide 250 mg (0.65 mmol) in THF—N-methylpyrrolidinone 10:1 (11 mL) was added NaBH₄ (99 mg). The mixture was stirred at r.t. overnight, then quenched with water. Upon extractive workup (EtOAc), silica gel chromatography purification afforded N-(((1S,9aS)-7-(methylamino)-3-oxo-1,3,9,9a-tetrahydrooxazolo[3,4-a]indol-1-yl)methyl)acetamide, which was directly used in the next step.

### Step 3. Preparation of N-((1S,9aS)-1-(acetamidomethyl)-3-oxo-1,3,9,9a-tetrahydrooxazolo[3,4-a]indol-7-yl)-2-chloro-N-methylacetamide

N-(((1S,9aS)-7-(methylamino)-3-oxo-1,3,9,9a-tetrahydrooxazolo[3,4-a]indol-1-yl)methyl)acetamide 90 mg (0.32 mmol) was dissolved in 1,4-dioxane (5 ml), excess aq. NaHCO₃ and chloroacetyl chloride 26 μl (d1.42, 0.32 mmol) was added. The resulting suspension was refluxed for 1 min. and left at r.t. for 2 h. The reaction was then quenched with water, extracted with EtOAc, and the crude product was purified with silica gel chromatography eluting with 80% EtOAc/hexane to afford N-((1S,9aS)-1-(acetamidomethyl)-3-oxo-1,3,9,9a-tetrahydrooxazolo[3,4-a]indol-7-yl)-2-chloro-N-methylacetamide. MS (m/z): 352.0 [M+H]⁺.

### Step 4. Preparation of compound Examples 26 and 27

A suspension of N-((1S,9aS)-1-(acetamidomethyl)-3-oxo-1,3,9,9a-tetrahydrooxazolo[3,4-a]indol-7-yl)-2-chloro-N-methylacetamide 110 mg (0.31 mmol) in toluene (2 ml) was flushed with N₂, followed by addition of Pd(OAc)₂ (14 mg, 0.06 mmol), and biphenyl-2-yldi-tert-butylphosphine (38 mg, 0.13 mmol). The reaction mixture was heated at 80° C. for 18 h, then directly purified by silica gel column chromatography eluting with EtOAc—hexanes 1:1. Two regioisomers were obtained with the compound in Example 27 being the major product.

Compound of Example 26: ¹H NMR (300 MHz, CD₃OD): 7.26 (s, 1H), 6.90 (s, 1H), 4.71˜4.65 (m, 1H), 4.58˜4.50 (m, 1H), 3.64 (d, J=4.80 Hz, 2H), 3.52 (m, 2H), 3.34˜3.21 (m, 2H), 3.17 (s, 3H), 2.01 (s, 3H). MS (m/z): 315.9 [M+H]⁺.

Compound of Example 27: ¹H NMR (300 MHz, CD₃OD): 7.22 (d, J=8.40 Hz, 1H), 6.82 (d, J=8.10 Hz, 1H), 4.70˜4.65 (m, 1H), 4.59˜4.52 (m, 1H), 3.64 (d, J=4.50 Hz, 2H), 3.47 (d, J=4.80 Hz, 2H), 3.25˜3.07 (m, 5H), 2.01 (s, 3H). MS (m/z): 315.9 [M+H]⁺.

### Example 28

### Preparation of (1S)-1-((1H-1,2,3-triazol-1-yl)methyl)-7-(6-(2-methyl-2H-tetrazol-5-yl)pyridin-3-yl)-9,9a-dihydrooxazolo[3,4-a]indol-3(1H)-one

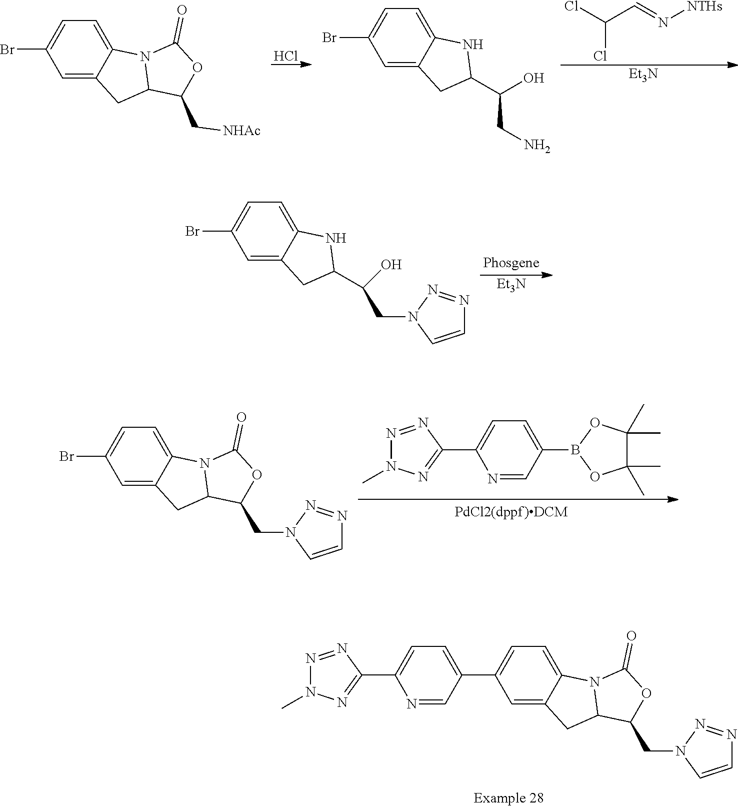

### Step 1. Preparation of (1S)-2-amino-1-(5-bromoindolin-2-yl)ethanol hydrochloride

N-(((1S,9aS)-7-bromo-3-oxo-1,3,9,9a-tetrahydrooxazolo[3,4-a]indol-1-yl)methyl)acetamide (1.0 g, 3.08 mmol) was suspended in conc. HCl (1.5 ml). The mixture was stirred at 90° C. for 4 h. The volatile was removed under reduced pressure. The crude product was taken directly into the next step. MS (m/z): 257.0 [M+H]⁺.

### Step 2. Preparation of (1S)-1-(5-bromoindolin-2-yl)-2-(1H-1,2,3-triazol-1-yl)ethanol

To the solution of (1S)-2-amino-1-(5-bromoindolin-2-yl)ethanol HCl salt in MeOH (30 ml) was added N′-(2,2-dichloroethylidene)-4-methylbenzenesulfonohydrazide (1.0 g, 3.55 mmol) and excess Et₃N (4.3 ml) at ca. 0° C. The mixture was allowed to warm up to r.t. and stirred overnight, then concentrated in vacuo. The residue was purified by silica gel chromatography eluting with 2-10% MeOH/DCM to afford (1S)-1-(5-bromoindolin-2-yl)-2-(1H-1,2,3-triazol-1-yl)ethanol. MS (m/z): 308.9 [M+H]⁺.

### Step 3. Preparation of (1S)-1-((1H-1,2,3-triazol-1-yl)methyl)-7-bromo-9,9a-dihydrooxazolo[3,4-a]indol-3(1H)-one

The solution of (1S)-1-(5-bromoindolin-2-yl)-2-(1H-1,2,3-triazol-1-yl)ethanol (0.5 g, 0.13 mmol), and Et₃N (2.1 ml, 15.1 mmol) in DCM (30 ml) was cooled at ca. 0° C., and phosgene in toluene (1.70 ml, 20%) was added. The mixture was allowed to warm up to r.t. and stirred overnight, then concentrated in vacuo. The two diastereomers were separated by preparative TLC eluting with EtOAc—hexanes 5:1.

For diastereomer A (R_{f}0.41): ¹H NMR (300 MHz, CD₃OD): 8.02 (d, J=1.20 Hz, 1H), 7.72 (d, J=0.90 Hz, 1H), 7.45˜7.37 (m, 2H), 7.21 (d, J=8.10 Hz, 1H), 5.36˜5.33 (m, 1H), 5.22˜5.13 (m, 1H), 3.57˜3.48 (m, 2H), 3.21˜3.10 (m, 2H). MS (m/z): 335.0 [M+H]⁺.

For diastereomer B: (R_{f} 0.44) ¹H NMR (300 MHz, CD₃OD): 8.10 (d, J=1.20 Hz, 1H), 7.79 (d, J=0.90 Hz, 1H), 7.42˜7.36 (m, 2H), 7.21 (d, J=8.40 Hz, 1H), 5.08˜5.33 (m, 1H), 4.77˜4.59 (m, 1H), 3.60˜3.49 (m, 2H), 3.18˜3.07 (m, 2H). MS (m/z): 335.0 [M+H]⁺.

### Step 4. Preparation of (1S,9aS)-1-((1H-1,2,3-triazol-1-yl)methyl)-7-(6-(2-methyl-2H-tetrazol-5-yl)pyridin-3-yl)-9,9a-dihydrooxazolo[3,4-a]indol-3(1H)-one

Each of diastereomers A or B from Step 3 were reacted with 2-(2-methyl-2H-tetrazol-5-yl)-5-(4,4,5,5-tetramethyl-1,3,2-dioxaborolan-2-yl)pyridine as described for Example 1 (Step 13) to produce two isomers of the Example 28.

Compound of Example 28, diastereomer A: MS (m/z): 416 [M+H]⁺.

Compound of Example 28, diastereomer B: MS (m/z): 416 [M+H]⁺.

### Example 29

### Preparation of N-(((1S,9aS)-7-(6-(1,2,3-triazol-yl)pyridin-3-yl)-3-oxo-1,3,9,9a-tetrahydrooxazolo[3,4-a]indol-1-yl)methyl)acetamide

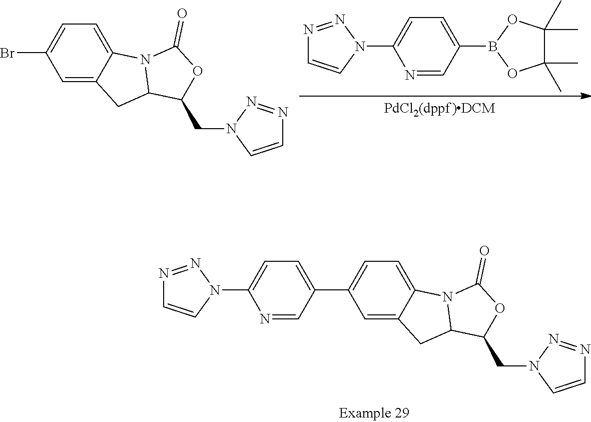

The compound of Example 29 was prepared according to the procedure of Example 16 (Step 13) by reacting N-(((1S,9aS)-3-oxo-7-(4,4,5,5-tetramethyl-1,3,2-dioxaborolan-2-yl)-1,3,9,9a-tetrahydrooxazolo[3,4-a]indol-1-yl)methyl)acetamide with 2-(1,2,3-triazol-1-yl)-5-bromopyridine (in place of the tetrazole derivative used for Example 16).

### Example 30

### Preparation of N-(((1S,9aS)-7-(6-(4-hydroxymethyl-1,2,3-triazol-yl)pyridin-3-yl)-3-oxo-1,3,9,9a-tetrahydrooxazolo[3,4-a]indol-1-yl)methyl)acetamide

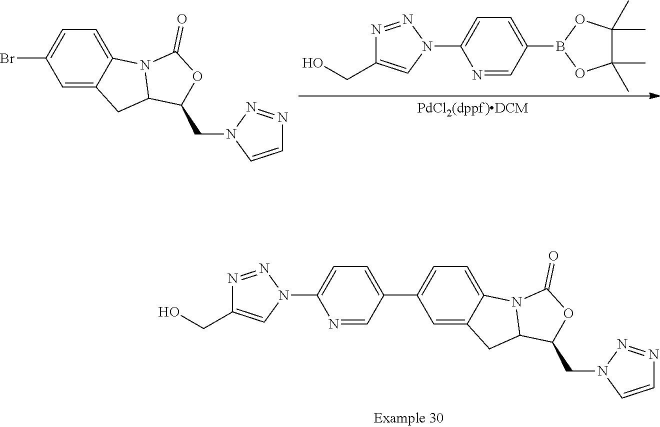

The compound of Example 30 was prepared according to the procedure of Example 16 (Step 13) by reacting N-(((1S,9aS)-3-oxo-7-(4,4,5,5-tetramethyl-1,3,2-dioxaborolan-2-yl)-1,3,9,9a-tetrahydrooxazolo[3,4-a]indol-1-yl)methyl)acetamide with 2-(4-hydroxymethyl-1,2,3-triazol-1-yl)-5-bromopyridine (in place of the tetrazole derivative used for Example 16).

### Example 31

### Preparation of N-(((1S,9aS)-3-oxo-7-(4-oxo-3,4-dihydropyridin-1(2H)-yl)-1,3,9,9a-tetrahydrooxazolo[3,4-a]indol-1-yl)methyl)acetamide

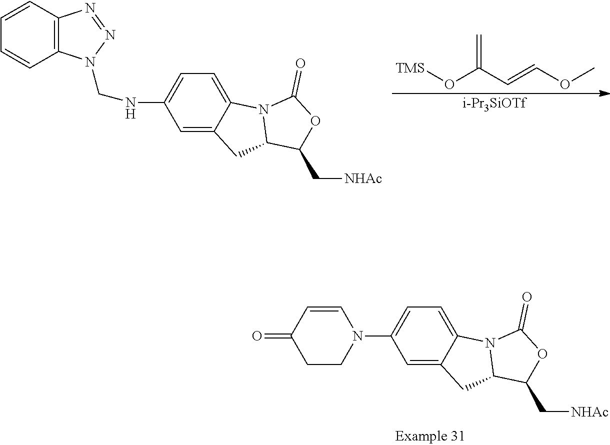

Danishefsky's diene (22 μl, 0.104 mmol) was added to a solution of N-(((1S,9aS)-7-((1H-benzo[d][1,2,3]triazol-1-yl)methylamino)-3-oxo-1,3,9,9a-tetrahydrooxazolo[3,4-a]indol-1-yl)methyl)acetamide (20 mg, 0.052 mmol; prepared as for Example 26, Step 1) in THF (100 μL) at ca. 0° C., followed by triisopropylsilyl triflate (ca. 20 μL). The mixture was kept for 0.5 h at 0° C., then warmed up to r.t. and kept at r.t. 2 h. The reaction mixture was carefully poured into sat. NaHCO₃ aq., and extracted with EtOAc. Solvent was removed under vacuum, and the residue was purified by prep. HPLC.

### Example 32

### Preparation of N-(((1S,9aS)-7-(6-(4-(2-hydroxypropan-2-yl)-1H-1,2,3-triazol-1-yl)pyridin-3-yl)-3-oxo-1,3,9,9a-tetrahydrooxazolo[3,4-a]indol-1-yl)methyl)acetamide

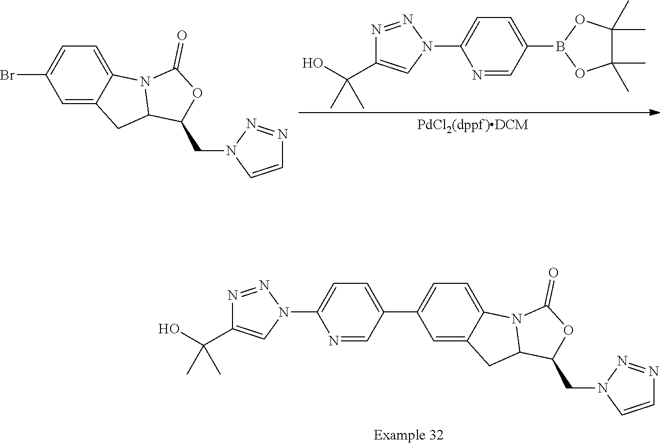

The compound of Example 32 was prepared according to the procedure of Example 16 (Step 13) by reacting N-(((1S,9aS)-3-oxo-7-(4,4,5,5-tetramethyl-1,3,2-dioxaborolan-2-yl)-1,3,9,9a-tetrahydrooxazolo[3,4-a]indol-1-yl)methyl)acetamide with 2-(4-(1-hydroxy-1-methyl)ethyl-1,2,3-triazol-1-yl)-5-bromopyridine (in place of the tetrazole derivative used for Example 16).

### Example 33

### Preparation of (1R,9aS)-3-oxo-7-(pyridin-3-yl)-1,3,9,9a-tetrahydrooxazolo[3,4-a]indole-1-carbonitrile

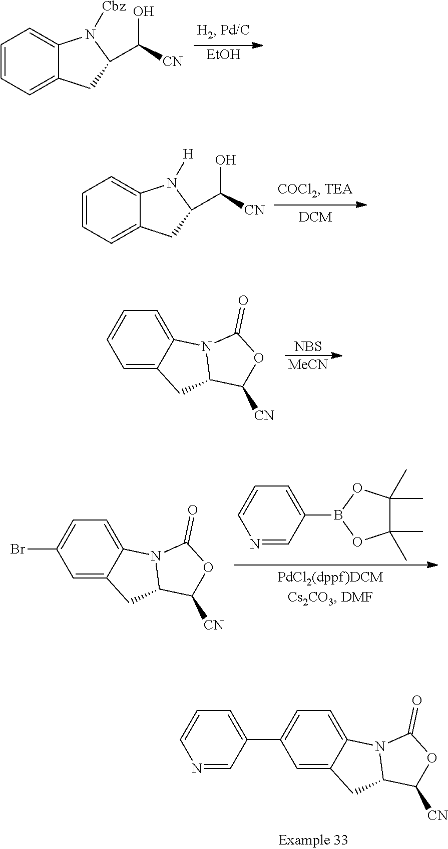

### Step 1. Preparation of (R)-2-hydroxy-2-((S)-indolin-2-yl)acetonitrile

(S)-benzyl 2-((R)-cyano(hydroxy)methyl)indoline-1-carboxylate (462 mg, 1.5 mmol) was dissolved in EtOH (25 mL), 5% Pd/C (139 mg) was added, and the flask was connected to the hydrogen source (1 atm). The mixture was stirred at r.t. for 3 h. Additional 5% Pd/C (220 mg) was added, and the hydrogenation continued for 7 h. The mixture was filtered through Celite. The solvent was removed under vacuum, and the product isolated by silica gel column chromatography (eluent: EtOAc—hexanes 1:3) as light-yellow crystals. MS (m/z): 175 [M+H]⁺.

### Step 2. Preparation of (1R,9aS)-3-oxo-1,3,9,9a-tetrahydrooxazolo[3,4-a]indole-1-carbonitrile

20% Phosgene in toluene (0.347 mL, 0.66 mmol) was added to the solution of the amino alcohol (77 mg, 0.44 mmol) from the preceding Step 1 and Et₃N (0.184 mL) in DCM (8.0 mL) was added dropwise with stirring at ca. 5-10° C. The mixture was allowed to warm up to r.t. over ca. 4 h. EtOAc (30 mL) was added, washed with water/brine (1:1; 3×15 mL), 10% citric acid (2×10 mL), brine, and dried (MgSO₄). Solvent was removed under vacuum, and the oxazolidinone product was isolated by column chromatography (eluent: EtOAc—hexanes 1:3). White crystals, yield 57 mg (65%). MS (m/z): 201 [M+H]⁺.

### Step 3. Preparation of (1R,9aS)-7-bromo-3-oxo-1,3,9,9a-tetrahydrooxazolo[3,4-a]indole-1-carbonitrile

N-Bromosuccinimide (45 mg, 0.25 mmol) was added to the oxazolidinone intermediate from the preceding Step 2 (43 mg, 0.21 mmol) in MeCN (2.0 mL) with stirring, the flask was flushed with nitrogen, and the solution was stirred at r.t. ca. 24 h. Solvent was removed under vacuum, and the product was purified by silica gel column chromatography (eluent:hexanes—EtOAc 4:1). White crystals, yield 53 mg (90%). MS (m/z): 279 [M+H]⁺.

### Step 4. Preparation of (1R,9aS)-3-oxo-7-(pyridin-3-yl)-1,3,9,9a-tetrahydrooxazolo[3,4-a]indole-1-carbonitrile

The mixture of the bromoaryl oxazolidinone reagent from the preceding Step 3 (28 mg, 0.1 mmol), 2-(3-pyridyl)-4,4,5,5-tetramethyl-1,3,2-dioxaborolane (31 mg, 0.15 mmol), Cs₂CO₃ (50 mg, 0.15 mmol), and PdCl₂(dppf)DCM (16 mg, 0.02 mmol) was degassed under N₂. Anh. DMF (1.0 mL) was added, and the mixture was sonicated briefly and then stirred at r.t. for 26 h. The reaction mixture was diluted w. EtOAc (ca. 3 mL) and filtered through Celite. Volatiles were removed under vacuum, and the crude product was purified by silica gel column chromatography (eluent: 1-2% MeOH in DCM w. 0.5% TEA).

Yield 15 mg (54%). MS (m/z): 278 [M+H]⁺.

### Example 34

### Preparation of N-(((1S,9aS)-7-(6-((1R,5S,6r)-6-cyano-3-oxabicyclo[3.1.0]hexan-6-yl)pyridin-3-yl)-3-oxo-1,3,9,9a-tetrahydrooxazolo[3,4-a]indol-1-yl)methyl)acetamide

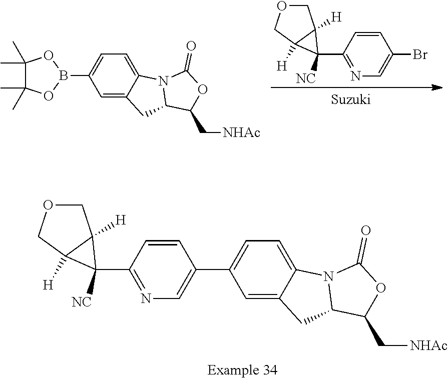

The compound of Example 34 was prepared according to the procedure of Example 17 by reacting N-(((1S,9aS)-3-oxo-7-(4,4,5,5-tetramethyl-1,3,2-dioxaborolan-2-yl)-1,3,9,9a-tetrahydrooxazolo[3,4-a]indol-1-yl)methyl)acetamide with (1R,5S,6r)-6-(5-bromopyridin-2-yl)-3-oxabicyclo[3.1.0]hexane-6-carbonitrile (in place of the azabicyclic pyridyl bromide used for Example 17).

### Example 35

### Preparation of ((2R,3S)-((((1S,9aS)-7-(6-(1H-tetrazol-1-yl)pyridin-3-yl)-3-oxo-1,3,9,9a-tetrahydrooxazolo[3,4-a]indol-1-yl)methyl)(acetyl)carbamoyloxy)methyl) 2-amino-3-methylpentanoate dihydrochloride

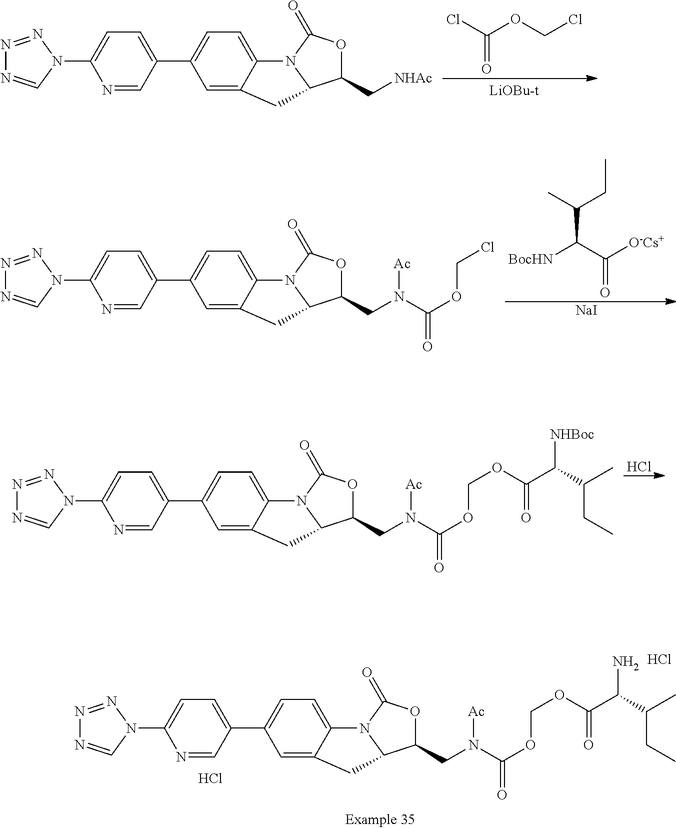

### Step 1. Preparation of chloromethyl(((1S,9aS)-7-(6-(1H-tetrazol-1-yl)pyridin-3-yl)-3-oxo-1,3,9,9a-tetrahydrooxazolo[3,4-a]indol-1-yl)methyl)(acetyl)carbamate

1M LiOBu-t in hexanes (0.55 mL, 0.55 mmol) was added dropwise with stirring to N-(((1S,9aS)-7-(6-(1H-tetrazol-1-yl)pyridin-3-yl)-3-oxo-1,3,9,9a-tetrahydrooxazolo[3,4-a]indol-1-yl)methyl)acetamide (182 mg, 0.5 mmol; Example 4) in MeCN (4 mL) and N-methylpyrrolidine-2-one (NMP, 2 mL) at ca. −10-0° C. under N₂ The mixture was stirred at this temperature for 30 min, then at 5-10° C. for ca. 10 min, and re-chilled to −10-0° C. Chloromethyl chloroformate (60 uL, 0.6 mmol) was added dropwise. The mixture was allowed to warm up to r.t. over ca. 1 h and stirred overnight. Most of volatiles were removed under vacuum, and EtOAc (60 mL) with water (15 mL) were added. Organic layer was washed with water, brine, and dried (MgSO₄). Solvent was removed under vacuum and the product isolated by silica gel column chromatograpy (1-5% MeOH in DCM).

### Step 2. Preparation of ((2R,3S)-((((1S,9aS)-7-(6-(1H-tetrazol-1-yl)pyridin-3-yl)-3-oxo-1,3,9,9a-tetrahydrooxazolo[3,4-a]indol-1-yl)methyl)(acetyl)carbamoyloxy)methyl) 2-(tert-butoxycarbonylamino)-3-methylpentanoate

A mixture of the compound from preceding Step 1 (0.5 mmol, 244 mg), N-Boc-isoleucine cesium salt (0.75 mmol), and NaI (0.5 mmol, 75 mg) in MeCN (12 mL) was stirred under refluxed overnight. Upon cooling to r.t., the mixture was filtered, and the precipitated salts washed with DCM. Filtrate was evaporated under vacuum, and EtOAc (50 mL) with water (15 mL) were added. Organic layer was washed with water, 10% aq. Na₂S₂O₃, water, brine, and dried (MgSO₄). The solvent was removed under vacuum, and the product isolated by siliga gel column chromatograpy (1-5% MeOH in DCM).

### Step 3. Preparation of ((2R,3S)-((((1S,9aS)-7-(6-(1H-tetrazol-1-yl)pyridin-3-yl)-3-oxo-1,3,9,9a-tetrahydrooxazolo[3,4-a]indol-1-yl)methyl)(acetyl)carbamoyloxy)methyl) 2-amino-3-methylpentanoate dihydrochloride

4M HCl in dioxane (2.5 mL, 10 mmol) was added dropwise with stirring to the compound from preceding Step 2 (0.25 mmol, 170 mg) with anisole (25 mg) in THF (2 mL) at 0° C. The mixture was allowed to warm up to r.t. overnight. Ether (10 mL) was added dropwise with stirring, the precipitated product filtered off, washed with ether, and dried under vacuum.

### Example 36

### Preparation of ((R)-(acetyl(((1S,9aS)-3-oxo-7-(6-(2-oxooxazolidin-3-yl)pyridin-3-yl)-1,3,9,9a-tetrahydrooxazolo[3,4-a]indol-1-yl)methyl)carbamoyloxy)methyl) 2-amino-3-methylbutanoate dihydrochloride

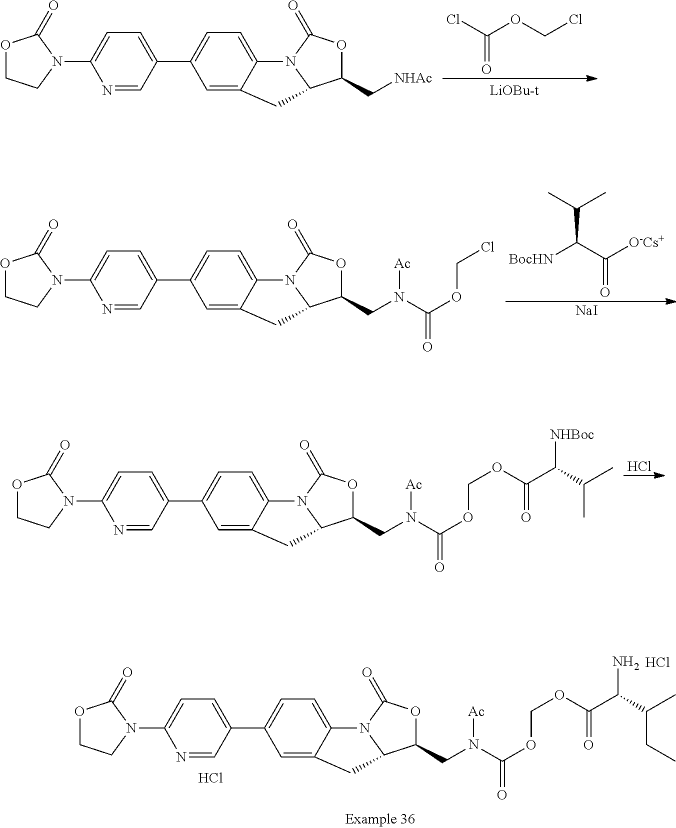

((R)-(acetyl(((1S,9aS)-3-oxo-7-(6-(2-oxooxazolidin-3-yl)pyridin-3-yl)-1,3,9,9a-tetrahydrooxazolo[3,4-a]indol-1-yl)methyl)carbamoyloxy)methyl) 2-amino-3-methylbutanoate dihydrochloride was prepared following the procedure for the compound of Example 35, except using N-(((1S,9aS)-3-oxo-7-(6-(2-oxooxazolidin-3-yl)pyridin-3-yl)-1,3,9,9a-tetrahydrooxazolo[3,4-a]indol-1-yl)methyl)acetamide in place of the compound of Example 4, and using Boc-valine cesium salt instead of Boc-isoleucine cesium salt used for Example 35.

### Utility and Testing

The compounds of the subject invention can exhibit potent activities against a variety of microorganisms, including gram positive microorganisms. Accordingly, the compounds of the subject invention can have broad antibacterial activity. Thus, compounds of the present invention can be useful antimicrobial agents and may be effective against a number of human and veterinary pathogens, including gram positive aerobic bacteria such as multiply-resistant staphylococci and streptococci, select gram negative microorganisms such as H. influenzae and M. catarrahlis, as well as anaerobic microorganisms such as bacteroides and clostridia species, and acid-fast microorganisms such as Mycobacterium tuberculosis and Mycobacterium avium.

The in vitro activity of compounds of the subject invention may be assessed by standard testing procedures such as the determination of minimum inhibitory concentration (MIC) by agar dilution as described in “Approved Standard. Methods for Dilution Antimicrobial Susceptibility Tests for Bacteria that Grow Aerobically,” 3^{rd} ed., published 1993 by the National Committee for Clinical Laboratory standards, Villanova, Pa., USA.

The in vitro MICs of test compounds may be determined by a standard agar dilution method. A stock drug solution of each analog is prepared in a preferred solvent, usually DMSO:H₂O (1:3). Serial 2-fold dilutions of each sample are made using 1.0 ml aliquots of sterile distilled water. To each 1.0 ml aliquot of drug is added 9 ml of molten Mueller Hinton agar medium. The drug-supplemented agar is mixed, poured into 15×100 mm petri dishes, and allowed to solidify and dry prior to inoculation.

Vials of each of the test microorganisms are maintained frozen in the vapor phase of a liquid nitrogen freezer. Test cultures are grown overnight at 35° C. on the medium appropriate for the microorganism. Colonies are harvested with a sterile swab, and cell suspensions are prepared in Trypticase Soy broth (TSB) to equal the turbidity of a 0.5 McFarland standard. A 1:20 dilution of each suspension is made in TSB. The plates containing the drug supplemented agar are inoculated with a 0.001 ml drop of the cell suspension using a Steers replicator, yielding approximately 10⁴ to 10⁵ cells per spot. The plates are incubated overnight at 35° C.

Following incubation the Minimum Inhibitory Concentration (MIC μg/ml), the lowest concentration of drug that inhibits visible growth of the microorganism, is read and recorded.

### Administration and Pharmaceutical Formulations

In general, the compounds of the subject invention can be administered in a therapeutically effective amount by any of the accepted modes of administration for agents that serve similar utilities. By way of example, the compounds of the subject invention may be administered orally, parenterally, transdermally, topically, rectally, or intranasally. The actual amount of the compound of the subject invention, i.e., the active ingredient, will depend on a number of factors, such as the severity of the disease, i.e., the infection, to be treated, the age and relative health of the subject, the potency of the compound used, the route and form of administration, and other factors, all of which are within the purview of the attending clinician.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range which includes the IC₅₀ (i.e., the concentration of the test compound which achieves a half-maximal inhibition of symptoms) as determined in cell culture Such information can be used to more accurately determine useful doses in humans. Levels in plasma may be measured, for example, by high performance liquid chromatography.

When employed as pharmaceuticals, the compounds of the subject invention are usually administered in the form of pharmaceutical compositions. These compounds can be administered by a variety of routes including oral, parenteral, transdermal, topical, rectal, and intranasal.

These compounds are effective as both injectable and oral compositions. Such compositions are prepared in a manner well known in the pharmaceutical art and comprise at least one active compound.

This invention also includes pharmaceutical compositions which contain, as the active ingredient, one or more of the compounds of the subject invention above associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The quantity of active component, that is the compound according to the subject invention, in the pharmaceutical composition and unit dosage form thereof may be varied or adjusted widely depending upon the particular application, the potency of the particular compound and the desired concentration.

The compositions are preferably formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Preferably, the compound of the subject invention above is employed at no more than about 20 weight percent of the pharmaceutical composition, more preferably no more than about 15 weight percent, with the balance being pharmaceutically inert carrier(s).

The active compound is effective over a wide dosage range and is generally administered in a pharmaceutically or therapeutically effective amount. It, will be understood, however, that the amount of the compound actually administered will be determined by a physician, in the light of the relevant circumstances, including the condition to be treated, the severity of the bacterial infection being treated, the chosen route of administration, the actual compound administered, the age, weight, and response of the individual patient, the severity of the patient's symptoms, and the like.

In therapeutic use for treating, or combating, bacterial infections in warm-blooded animals, the compounds or pharmaceutical compositions thereof will be administered orally, topically, transdermally, and/or parenterally at a dosage to obtain and maintain a concentration, that is, an amount, or blood-level of active component in the animal undergoing treatment which will be antibacterially effective. Generally, such antibacterially or therapeutically effective amount of dosage of active component (i.e., an effective dosage) will be in the range of about 0.1 to about 100, more preferably about 1.0 to about 50 mg/kg of body weight/day.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure-breathing machine. Solution, suspension, or powder compositions may be administered, preferably orally or nasally, from devices that deliver the formulation in an appropriate manner.

The following formulation examples illustrate representative pharmaceutical compositions of the present invention.

### Formulation Example 1

Hard gelatin capsules containing the following ingredients are prepared:

| Ingredient | Amount (mg/capsule) |
|---|---|
| Active Ingredient | 30.0 mg |
| Starch | 305.0 mg |
| Magnesium stearate | 5.0 mg |

The above ingredients are mixed and filled into hard gelatin capsules in 340 mg quantities.

### Formulation Example 2

A tablet formula is prepared using the ingredients below:

| Ingredient | Amount (mg/tablet) |
|---|---|
| Active Ingredient | 25.0 mg |
| Cellulose, microcrystalline | 200.0 mg |
| Colloidal silicon dioxide | 10.0 mg |
| Stearic acid | 5.0 mg |

The components are blended and compressed to form tablets, each weighing 240 mg.

### Formulation Example 3

A dry powder inhaler formulation is prepared containing the following components:

| Ingredient | Weight % |
|---|---|
| Active Ingredient | 5 |
| Lactose | 95 |

The active ingredient is mixed with the lactose and the mixture is added to a dry powder inhaling appliance.

### Formulation Example 4

Tablets, each containing 30 mg of active ingredient, are prepared as follows:

| Ingredient | Amount (mg/tablet) |
|---|---|
| Active Ingredient | 30.0 mg |
| Starch cellulose | 45.0 mg |
| Microcrystalline cellulose | 35.0 mg |
| Polyvinylpyrrolidone (as 10% | 4.0 mg |
| solution in sterile water) | |
| Sodium carboxymethyl starch | 4.5 mg |
| Magnesium stearate | 0.5 mg |
| Talc | 1.0 mg |
| Total | 1.20 mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### Formulation Example 5

Capsules, each containing 40 mg of medicament are made as follows:

| Ingredient | Amount (mg/capsule) |
|---|---|
| Active Ingredient | 40.0 mg |
| Starch | 109.0 mg |
| Magnesium stearate | 1.0 mg |
| Total | 150.0 mg |

The active ingredient, starch and magnesium stearate are blended, passed through a No. 20 mesh U.S. sieve, and filled into hard gelatin capsules in 150 mg quantities.

### Formulation Example 6

Suppositories, each containing 25 mg of active ingredient are made as follows:

| Ingredient | Amount |
|---|---|
| Active Ingredient | 25 mg |
| Lactose | 2,000 mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation Example 7

Suspensions, each containing 50 mg of medicament per 5.0 mL dose are made as follows:

| Ingredient | Amount |
|---|---|
| Active Ingredient | 50.0 mg |
| Xanthan gum | 4.0 mg |
| Sodium carboxymethyl cellulose | 50.0 mg |
| (11%); Microcrystalline | |
| cellulose (89%) | |
| Sucrose | 1.75 mg |
| Sodium benzoate | 10.0 mg |
| Flavor and Color | q.v. |
| Purified water to | 5.0 mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation Example 8

| Ingredient | Quantity (per capsule) |
|---|---|
| Active Ingredient | 15.0 mg |
| Starch | 407.0 mg |
| Magnesium stearate | 3.0 mg |
| Total | 425.0 mg |

The active ingredient, starch, and magnesium stearate are blended, passed through a No. 20 mesh U.S. sieve, and filled into hard gelatin capsules in 425.0 mg quantities.

### Formulation Example 9

A subcutaneous formulation may be prepared as follows:

| Ingredient | Quantity |
|---|---|
| Active Ingredient | 5.0 mg |
| Corn Oil | 1.0 mL |

### Formulation Example 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | To 100 | g |

The white soft paraffin is heated until molten. The liquid paraffin and emulsifying wax are incorporated and stirred until dissolved. The active ingredient is added and stirring is continued until dispersed. The mixture is then cooled until solid.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 which is herein incorporated by reference.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions that can transiently open the blood-brain barrier.

Other suitable formulations for use in the present invention can be found in Remington's Pharmaceutical Sciences, Mace Publishing Company, Philadelphia, Pa., 17th ed. (1985).

As noted above, the compounds described herein are suitable for use in a variety of drug delivery systems described above. Additionally, in order to enhance the in vivo serum half-life of the administered compound, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. Nos. 4,235,871, 4,501,728 and 4,837,028 each of which is incorporated herein by reference.

As noted above, the compounds administered to a patient are in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 and 8. It will be understood that use of certain of the foregoing excipients, carriers, or stabilizers will result in the formation of pharmaceutical salts.

Compounds of this invention can have useful activity against a variety of pathogenic microorganisms. The in vitro activity of compounds of this invention can be assessed by standard testing procedures such as the determination of minimum inhibitory concentration (MIC) by agar dilution as described in “Approved Standard. Methods for Dilution Antimicrobial Susceptibility Tests for Bacteria That Grow Aerobically”, 3rd. ed., published 1993 by the National Committee for Clinical Laboratory Standards, Villanova, Pa., USA. Minimum inhibitory concentration (MIC) refers to the lowest concentration of drug (μg/mL) that inhibits visible growth of the organism. Lower MIC values indicate a higher antibacterial activity. Typically, the compounds of present invention have useful potency against Gram-positive or Gram-negative pathogens with MIC values of at least 16 μg/mL or less. The activity of compounds of present invention against a clinical isolate of methicillin-resistant Staphylococcus aureus (MRSA; from the Massachusetts General Hospital, USA) is exemplified by the MIC data of Table 1.

TABLE 1
Antibacterial Activity (MIC μg/mL) and MAO A Inhibition (MAO-I)

| | MRSA, | MAO-I |
|---|---|---|
| EXAMPLES | MIC, μg/mL | IC₅₀, μg/mL |
| Linezolid | 2.0 | 5 |
| 1 | 0.5 | — |
| 2 | 0.5 | — |
| 3 | 0.25 | — |
| 4 | 0.5 | 138 |
| 6 | 0.5 | — |
| 8 | 1.0 | — |
| 11 | 1.0 | >100 |
| 18 | 0.5 | >100 |

Human monoamine oxidase (MOA) A type enzyme inhibition activity for select compounds was measured using a commercial MAO assay kit MAO-Glo™ from Promega Co. (USA). The assay was performed as described in the company's technical bulletin MAOGlo™ Assay”. The protocol involves an incubation of the MAO A enzyme with a luminogenic MAO substrate to produce an enzymatic product which is converted to luciferin by a coupled reaction. The released luciferin undergoes further transformation to generate light that is detected and measured. The amount of the light is directly proportional to the activity of MAO. Percent inhibition at several concentrations is established relative to the uninhibited control rate, and the IC₅₀ values are calculated. A low IC₅₀ value indicates that the tested inhibitor possesses a strong affinity or binding to MAO enzyme, thus being a stronger inhibitor, as compared to the compound with a higher IC₅₀ value. The MAO inhibition data for select compound of this invention are illustrated in the Table 1.

1. A compound according to formula I 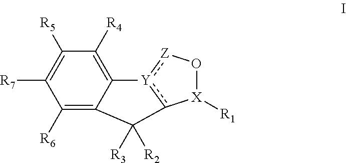
or a pharmaceutically acceptable salt thereof wherein:
R¹ is CH₂NHCOR′;
X is CH;
Y is N;
Z is C═O;
R² and R³ are independently H or F;
R⁴, R⁵, and R⁶ are independently H, F, Cl, CN, CH₃, or OH;
R⁷ is 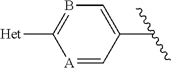
wherein R′ is selected from
H;
C₁₋₁₂alkyl,
C₁₋₁₂alkyl optionally substituted with 1-3Cl;
CH₂OH;
CH₂OC₁₋₁₂alkyl;
C₃₋₁₂cycloalkyl;
phenyl;
phenyl optionally substituted with 1-3 of groups OH, OMe, OEt, NO₂, halo, COOH, SO₃H, or NR″R′″, wherein R″ and R′″ are selected from H or C₁₋₁₂alkyl;
furanyl;
tetrahydrofuranyl;
2-thiophene;
pyrrolidinyl;
pyridinyl;
NH₂;
NHC₁₋₁₂alkyl;
NHPh;
COPh;
A and B are independently N or C—R¹², or C—R¹³; Het is Het₁ or Het₂; and R⁹, R¹⁰, R¹¹, R¹², R¹³ are independently H, halo, F, CN, CH₃, or OH; and
when R² is H, R³ is H, and R⁴ is H; then R⁷ is other than 3-pyridyl or 3-pyridyl substituted with H, C₁₋₄alkyl, NO₂, NH₂, NHC(═O)C₁₋₄alkyl, CN, COOH, halo, or N-oxides thereof.

2. The compound of claim 1 selected from:

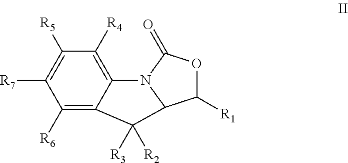

3. The compound of claim 1 selected from:

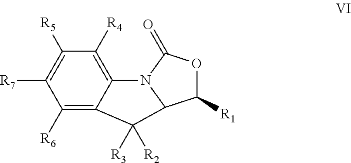

4. The compound of claim 1 selected from:

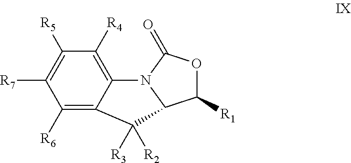

5. The compound of claim 1 according to formula XII:

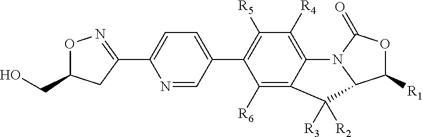

6. The compound of claim 5 according to one of the following:

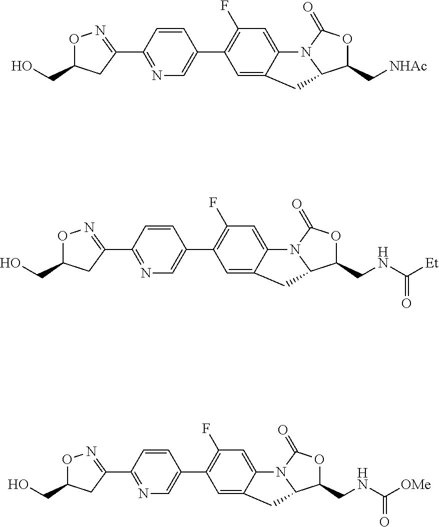

7. (canceled)

8. The compound of claim 1 according to one of the following:

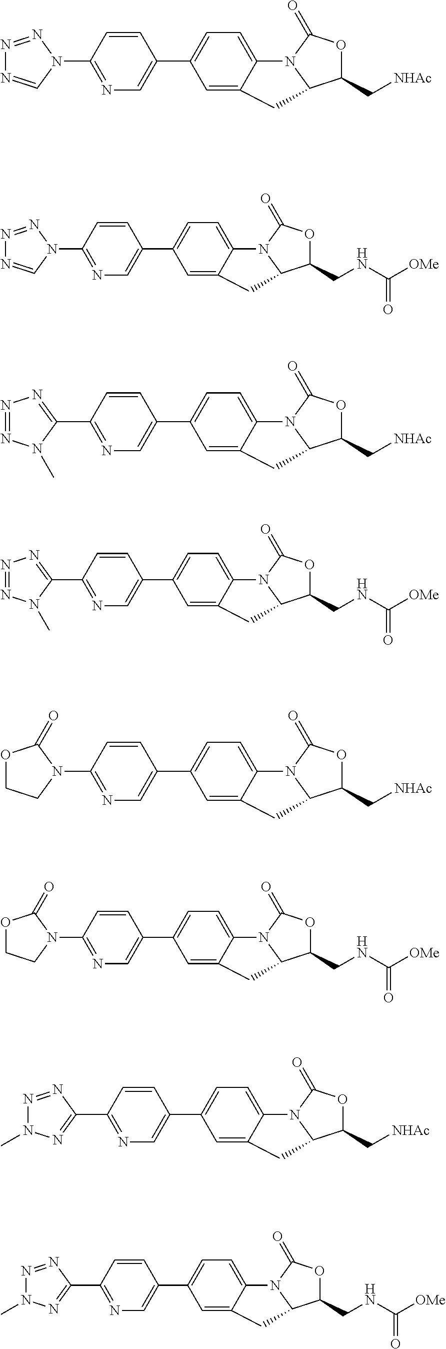

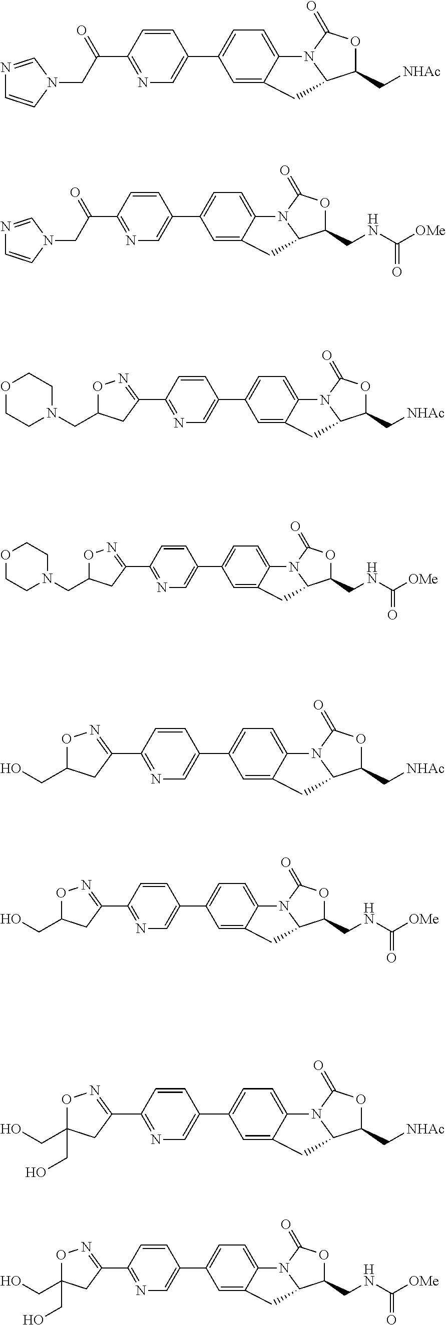

9.-10. (canceled)

11. The compound of claim 1 according to one of the following:

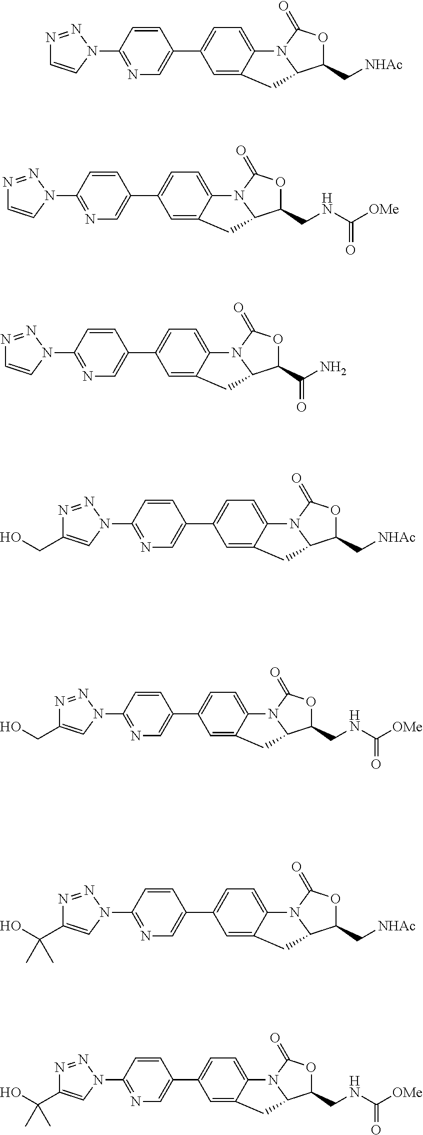

12. The compound of claim 1 according to one of the following:

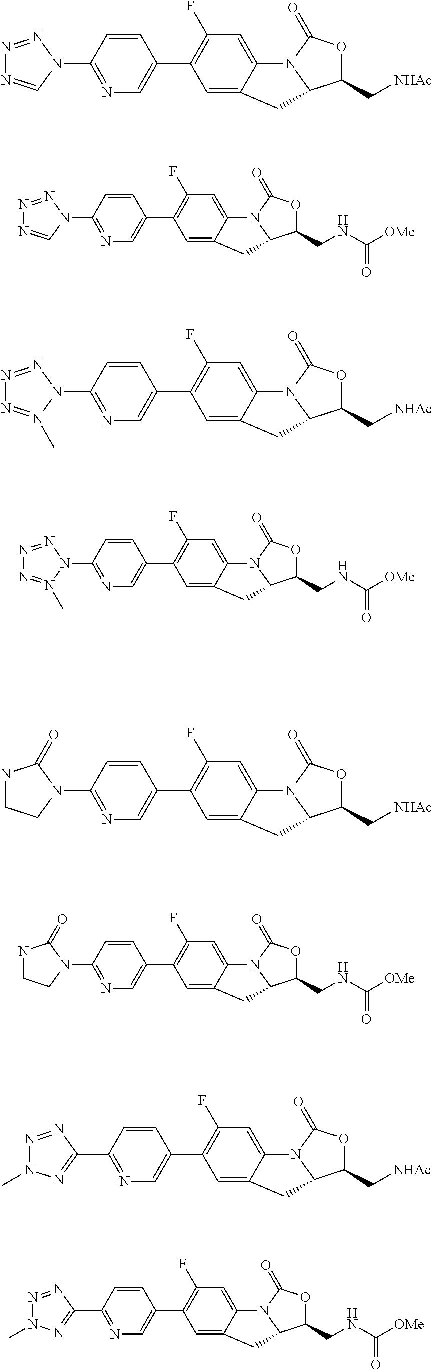

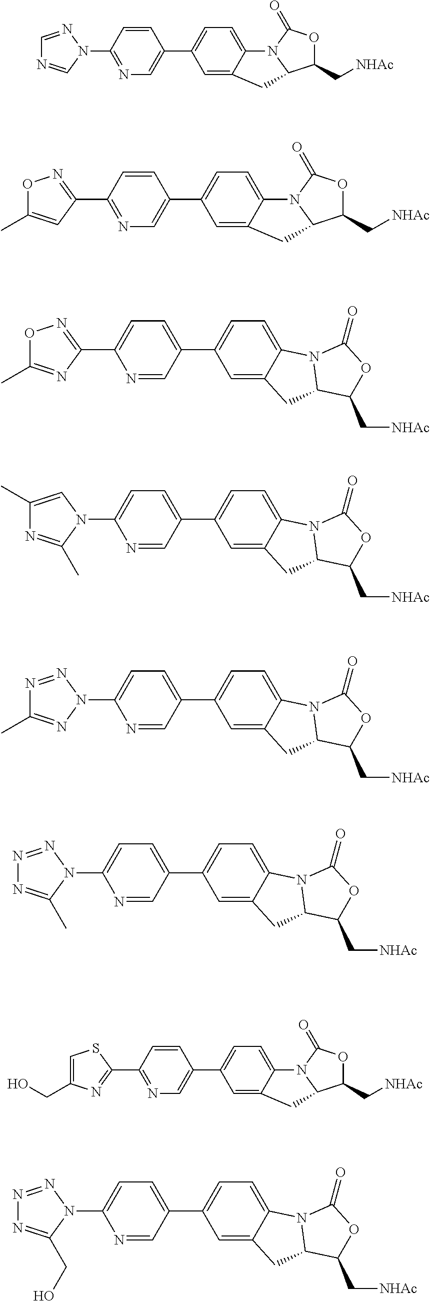

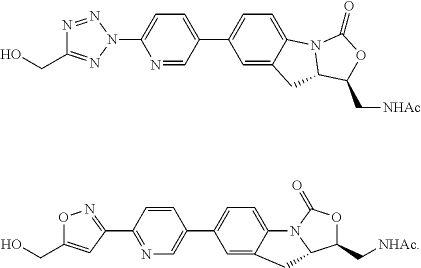

13. The compound of claim 1 according to one of the following:

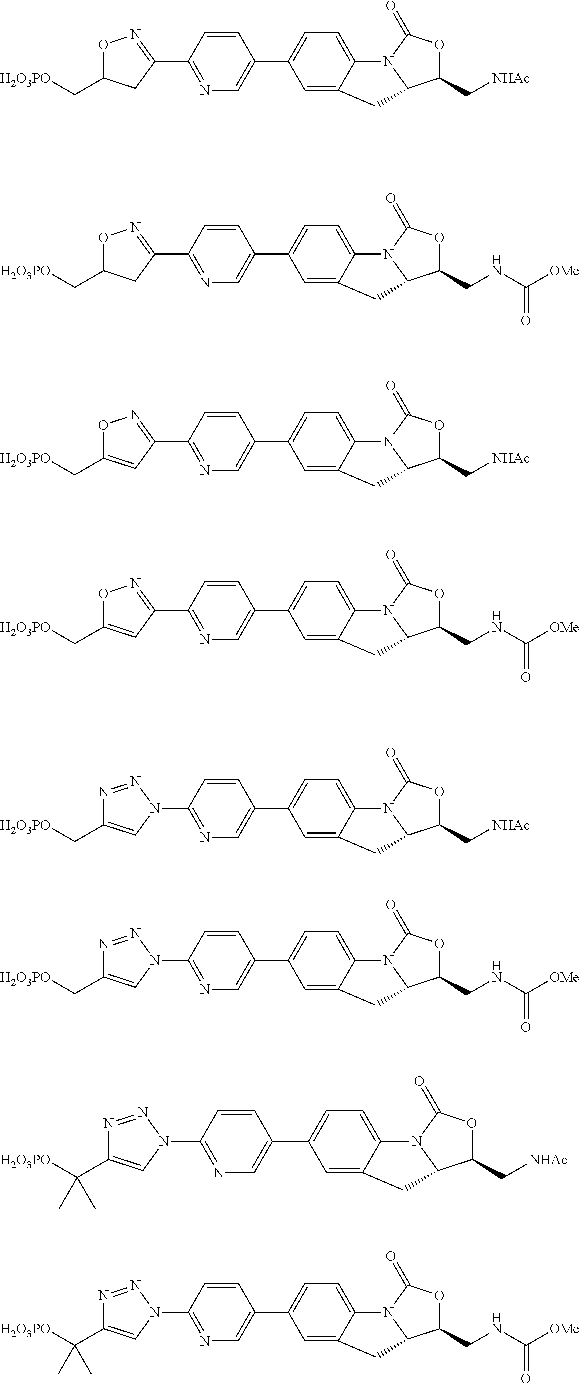

14.-65. (canceled)

66. A pharmaceutical composition comprising a therapeutically effective amount of a compound of claim 1 and a pharmaceutically acceptable carrier, excipient, or diluent.

67. The compound of claim 1 with a reduced monoamine oxidase inhibition over the antibacterial oxazolidinone therapy standard linezolid.

68.-86. (canceled)
